(19) United States
(12) Patent Application Publication
(10) Pub. No.: US 2010/0167178 A1
(43) Pub. Date: Jul. 1, 2010

(54) Oxime sulfonates and the use thereof as latent acids

(76) Inventors: Hitoshi Yamato, Hyogo (JP); Toshikage Asakura, Osaka (JP); Yuichi Nishimae, Osaka (JP); Takeshi Iwai, Kanagawa-ken (JP); Makiko Irie, Kanagawa-ken (JP); Kazuhiko Nakayama, Beaverton, OR (US)

Correspondence Address: BASF Performance Products LLC;Patent Department, 540 White Plains Road, P.O. Box 2005, Tarrytown, NY, 10591, US

(21) Appl. No.: 12/308,279

(22) PCT Filed: Jun. 15, 2007
(86) PCT No.: PCT/EP2007/055936

§ 371 Date: Dec. 11, 2008

(30) Foreign Application Priority Data
Jun. 20, 2006 (EP) 06115691.5

(51) Int. Cl. C07C 69/52; C08F 24/00; C08F 228/02; G03C 1/04; C08F 2/46; G03F 5/00; G03F 7/00

(52) U.S. Cl. 430/2; 560/221; 526/270; 526/287; 430/285.1; 522/59; 430/7

(57) ABSTRACT

Compounds of the formula (I), (II) or (III), wherein R₁ is for example C₁-C₁₈alkylsulfonyl, C₁-C₁₀haloalkylsulfonyl, camphorylsulfonyl, phenyl-C₁-C₃alkylsulfonyl, phenylsulfonyl, naphthylsulfonyl, anthrylsulfonyl, phenanthrylsulfonyl or heteroarylsulfonyl, R′₁ is for example phenylenedisulfonyl, R₂ is for example CN, C₁-C₁₀haloalkyl or C₁-C₁₀haloalkyl which is substituted by (IV); Ar₁ is for example phenyl optionally substituted by a group of formula (IV); Ar′₁ is for example phenylene which optionally is substituted by a group of formula (IV); A₁, A₂ and A₃ independently of each other are for example hydrogen, halogen, CN, or C₁-C₁₈alkyl; D₂ is for example a direct bond, O, (CO)O, (CO)S, SO₂, OSO₂ or C₁-C₁₈alkylene; or A₃ and D₂ together form C₃-C₃₀cycloalkenyl; or A₂ and D₂ together with the carbon of the ethylenically unsaturated double bond to which they are attached form C₃-C₃₀cycloalkyl; D₃ and D₄ for example independently of each other are a direct bond, O, S, C₁-C₁₈alkylene or C₃-C₃₀cycloalkylene provided that at least one of the radicals R₂, Ar₁ or Ar′₁ comprises a group of the formula (IV); are suitable as photolatent acid donors and for the preparation of corresponding polymers to be employed in chemically amplified photoresists.

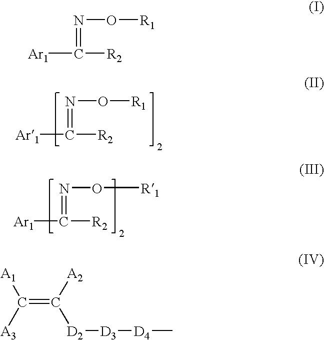

The invention relates to new oxime sulfonates bearing polymerizable ethylenically unsaturated groups, polymers comprising repeating units derived from the said compounds, chemically amplified photoresist compositions comprising said compounds and/or said polymers and to the use of the compounds and/or polymers as latent acids, which can be activated by irradiation with actinic electromagnetic radiation and electron beams.

In U.S. Pat. No. 4,540,598 surface-coating compositions comprising photosensitive oxime sulfonate compounds, e.g. 4-chloro-α-trifluoroacetophenonoxime benzenesulfonate and customary acid-curable resins are disclosed. In U.S. Pat. No. 5,627,011 and U.S. Pat. No. 5,759,740 the use of α-(4-toluene-sulfonyloxyimino)-4-methoxybenzyl cyanide and α-(4-toluene-sulfonyl-oxyimino)-3-thienylmethyl cyanide as latent acid catalysts in chemically amplified positive and negative photoresists for wavelengths of 340-390 nm, especially those in the radiation region of the mercury i line (365 nm) is described. In GB 2306958 the use of oxime-sulfonates as latent acid donors in positive and negative photoresists for wavelengths between 180 and 600 nm, especially those in the radiation region beyond 390 nm is reported. In U.S. Pat. No. 5,714,625 non aromatic α-(alkylsulfonyloxyimino)-1-cyclohexenylacetonitriles and α-(alkylsulfonyloxyimino)-1-cyclopentenylacetonitriles are disclosed. In EP 241423-oxime sulfonate compounds are employed in about 25% concentration as photolatent acid generators in non-chemically amplified positive resists. In U.S. Pat. No. 6,261,738 and WO 02/025376 oxime sulfonate compounds are described as latent acid donors in positive and negative photoresists particular suitable for applications in the Deep UV range. In EP 199672 (U.S. Pat. No. 4,736,055) oxime sulfonated bearing a polymerizable unsaturated group and polymers comprising said oxime sulfonated are described as component for photoresists. In U.S. Pat. No. 5,213,946, JP10-221852-A, JP11-218926-A, polymers with attached oxime sulfonate groups for chemically amplified compositions are disclosed, in which said oxime sulfonate groups are fixed to the polymer chain via the sulfonate moiety. In JP05-19477-A polymers with attached oxime sulfonate groups, attached via the chromophor moiety are described for non-chemically amplified compositions.

In the art exists a need for reactive non-ionic latent acid donors that are thermally and chemically stable and that, after being activated by light, UV-radiation, X-ray irradiation or electron beams can be used as catalysts for a variety of acid-catalysed reactions, such as polycondensation reactions, acid-catalysed depolymerisation reactions, acid-catalysed electrophilic substitution reactions or the acid-catalysed removal of protecting groups. A particular need exists for latent acid catalysts with high stability, high sensitivity and high resolution not only in the Deep-UV range but also in a wide range of wavelengths such as for example g-line (436 nm), i-line (365 nm), KrF (248 nm), ArF (193 nm) and EUV (13.5 nm). In addition, a new need emerges for latent acid catalysts with non-leaching properties with respect to the water medium, especially for immersion lithography, wherein the photoresist layer is immersed in water during exposure.

Surprisingly, it has now been found that specific oxime sulfonates and polymers attaching oxime sulfonates via the chromophor moiety, as described below, are stable and highly active against the wide range of light source. The oxime sulfonates and polymers attaching oxime sulfonates via the chromophor moiety in the present invention are especially suitable as catalysts for the aforementioned acid catalyzed reactions in chemically amplified photoresist applications. In addition, the oxime sulfonates and polymer with oxime sulfonates attached via the chromophor moiety in the present invention are suitable for immersion lithography due to their non-leaching properties to water medium. Furthermore, chemically amplified photoresist compositions comprising oxime sulfonates and polymer with oxime sulfonates attached via the chromophor moiety of the present invention are thermally stable, even at high bake temperatures during processing and they provide a high photospeed.

Subject of the invention is a compound of the formula I, II or III

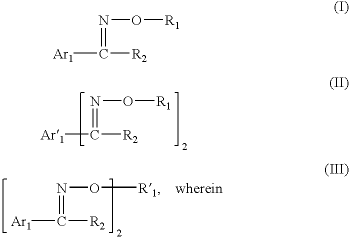

R₁ is C₁-C₁₈alkylsulfonyl, C₁-C₁₀haloalkylsulfonyl,
C₂-C₁₂alkenylsulfonyl, C₂-C₁₂alkynylsulfonyl, C₃-C₃₀cycloalkylsulfonyl, C₁-C₁₀alkylsulfonyl interrupted by one or more O or C₁-C₁₀haloalkylsulfonyl interrupted by one or more O;
wherein the groups C₁-C₁₈alkylsulfonyl, C₁-C₁₀haloalkylsulfonyl, C₂-C₁₂alkenylsulfonyl, C₂-C₁₂alkynylsulfonyl, C₃-C₃₀cycloalkylsulfonyl, interrupted C₁-C₁₈alkylsulfonyl and interrupted C₁-C₁₀haloalkylsulfonyl optionally are substituted by one or more C₃-C₃₀cycloalkyl, C₄-C₃₀cycloalkenyl, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂; or are substituted by NO₂, CN, Ar₂, (CO)R₇, (CO)OR′₃, (CO)NR₄R₅, O(CO)R₇, O(CO)OR′₃, O(CO)NR₄R₅, NR₆(CO)R₇, NR₆(CO)OR′₃, OR′₃, NR₄R₅, SR₆, SOR₇, SO₂R₇ and/or OSO₂R₇; or R₁ is camphorylsulfonyl, phenyl-C₁-C₃alkylsulfonyl, phenylsulfonyl, naphthylsulfonyl, anthrylsulfonyl, phenanthrylsulfonyl or heteroarylsulfonyl,
wherein the groups camphorylsulfonyl, phenyl-C₁-C₃alkylsulfonyl, phenylsulfonyl, naphthylsulfonyl, anthrylsulfonyl, phenanthrylsulfonyl and heteroarylsulfonyl optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆ CO, SO and/or SO₂, C₃-C₃₀cycloalkyl which is interrupted by one or more O, S, NR₆ CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂; or are substituted by halogen, NO₂, CN, Ar₂, (CO)R₇, (CO)OR′₃, (CO)NR₄R₅, O(CO)R₇, O(CO)OR′₃, O(CO)NR₄R₅, NR₆(CO)R₇, NR₆(CO)OR′₃, OR′₃, NR₄R₅, SR₆, SOR₇, SO₂R₇ and/or OSO₂R₇;
wherein all radicals R₁ optionally additionally are substituted by a group having a —O—C-bond or a —O—Si-bond which cleaves upon the action of an acid;
R′₁ is phenylenedisulfonyl, naphthylenedisulfonyl,

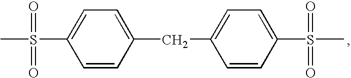

diphenylenedisulfonyl or oxydiphenylenedisulfonyl,
wherein the groups phenylenedisulfonyl, naphthylenedisulfonyl,

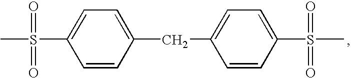

diphenylenedisulfonyl and oxydiphenylenedisulfonyl optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or are substituted by halogen, NO₂, CN, Ar₂, (CO)R₇, (CO)OR′₃, (CO)NR₄R₅, O(CO)R₇, O(CO)OR¹³, O(CO)NR₄R₅, NR₆(CO)R₇, NR₆(CO)OR′₃, OR′₃, NR₄R₅, SR₆, SOR₇, SO₂R₇ and/or OSO₂R₇;
or R′₁ is C₁-C₁₂alkylenedisulfonyl or C₁-C₁₀haloalkylenedisulfonyl;
wherein all radicals R′₁ optionally additionally are substituted by a group having a —O—C-bond or a —O—Si-bond which cleaves upon the action of an acid;
R₂ is CN, C₁-C₁₀haloalkyl or C₁-C₁₀haloalkyl which is substituted by NO₂, CN, Ar₂, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇, OSO₂R₇ and/or a group of formula IV

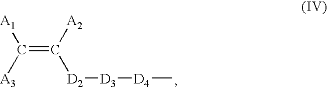

Ar₁ is phenyl, biphenylyl, fluorenyl, naphthyl, anthryl, phenanthryl or heteroaryl, wherein the groups phenyl, biphenylyl, fluorenyl, naphthyl, anthryl, phenanthryl and heteroaryl optionally are substituted by one or more C₃-C₃₀cycloalkyl, C₁-C₁₈ alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂; or are substituted by a group of formula IV

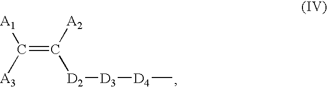

halogen, NO₂, CN, Ar₂, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇;
optionally the substituents C₁-C₁₈alkyl, C₂-C₁₂alkenyl, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇ form 5-, 6- or 7-membered rings, via the radicals C₁-C₁₈alkyl, C₂-C₁₂alkenyl, R₃, R₄, R₅, R₆ and/or R₇, with further substituents on the phenyl, biphenylyl, naphthyl, anthryl, phenanthryl, or heteroaryl ring or with one of the carbon atoms of the phenyl, biphenylyl, naphthyl, anthryl, phenanthryl, or heteroaryl ring;
wherein all radicals Ar₁ optionally additionally are substituted by a group having a —O—C-bond or a —O—Si-bond which cleaves upon the action of an acid;
Ar′₁ is phenylene, biphenylene, naphthylene,

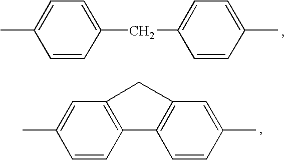

heteroarylene, oxydiphenylene or

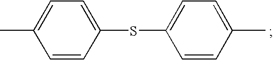

wherein the groups phenylene, biphenylene, naphthylene,

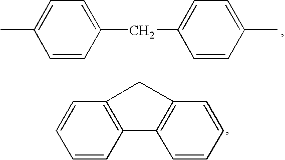

heteroarylene, oxydiphenylene and

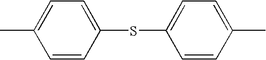

optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂; or are substituted by a group of formula IV

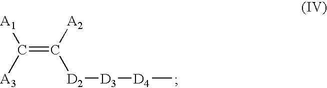

or are substituted by halogen, NO₂, CN, Ar₂, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇,

optionally the substituents C₁-C₁₈alkyl, C₂-C₁₂alkenyl, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇ form 5-, 6- or 7-membered rings, via the radicals C₁-C₁₈alkyl, C₂-C₁₂alkenyl, R₃, R₄, R₅, R₆ and/or R₇, with further substituents on the phenylene, biphenylene, naphthylene,

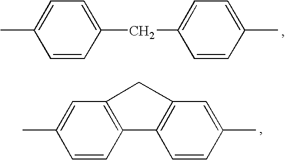

heteroarylene,

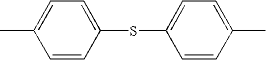

or oxydiphenylene ring or with one of the carbon atoms of the phenylene, biphenylene, naphthylene,

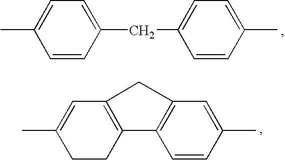

heteroarylene,

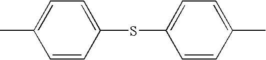

or oxydiphenylene ring;

### or Ar′₁ is —Ar″₁—X₁—Y₁—X₁—Ar″₁—;

wherein all radicals M₁ optionally additionally are substituted by a group having a —O—C-bond or a —O—Si-bond which cleaves upon the action of an acid,
Ar″₁ is phenylene, biphenylene, naphthylene, heteroarylene;
wherein the groups phenylene, biphenylene, naphthylene, heteroarylene optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂; or are substituted by halogen, NO₂, CN, Ar₂, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇,
optionally the substituents C₁-C₁₈alkyl, C₂-C₁₂alkenyl, (CO)R₇, (CO)OR₃, (CO)NR₄R₆, O(CO)R₇, O(CO)OR₃, O(CO)NR₄R₆, NR₆(CO)R₇, NR₆(CO)OR₃, OR₃, NR₄R₅, SR₆, SOR₇, SO₂R₇ and/or OSO₂R₇ form 5-, 6- or 7-membered rings, via the radicals C₁-C₁₈alkyl, C₂-C₁₂alkenyl, R₃, R₄, R₅, R₆ and/or R₇, with further substituents on the phenylene, biphenylene, naphthylene, heteroarylene ring or with one of the carbon atoms of the phenylene, biphenylene, naphthylene, heteroarylene ring;
wherein all radicals Ar″₁, optionally additionally are substituted by a group having a —O—C-bond or a —O—Si-bond which cleaves upon the action of an acid;
X₁ is a direct bond, O, S, NR₆, CO, O(CO), S(CO), NR₆(CO), SO, SO₂, or OSO₂; or X₁ is C₁-C₁₈alkylene or phenylene wherein these radicals are unsubstituted or substituted by one or more C₁-C₁₈alkyl, C₁-C₄haloalkyl, halogen, OR₃ and/or SR₆;
Y₁ is C₁-C₁₈alkylene which optionally is substituted by OR₃, SR₆, halogen, phenyl and/or a group of formula IV

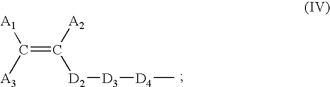

or Y₁ is C₂-C₁₈alkylene which is interrupted by one or more O, S, NR₆, CO, SO and/or SO₂ and which optionally is substituted by a group of formula IV

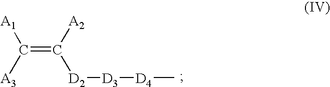

R′₃ is hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl;
or R′₃ is C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or R′3 is phenyl, naphthyl, C₂-C₁₈alkanoyl, benzoyl, C₁-C₁₈alkylsulfonyl, phenylsulfonyl, naphthylsulfonyl, anthrylsulfonyl or phenanthrylsulfonyl;
wherein the groups phenyl, naphthyl, C₂-C₁₈alkanoyl, benzoyl, C₁-C₁₈alkylsulfonyl, phenylsulfonyl, naphthylsulfonyl, anthrylsulfonyl and phenanthrylsulfonyl optionally are substituted by one or more Ar₂, OH, C₁-C₁₈alkyl, C₁-C₁₀haloalkyl, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy and/or benzoyloxy;
R₃ is C₃-C₃₀cycloalkyl, C₁-C₁₈alkyl, C₂-C₁₂alkenyl, C₄-C₃₀cycloalkenyl, phenyl-C₁-C₃-alkyl; all of which optionally are substituted by a group of formula IV

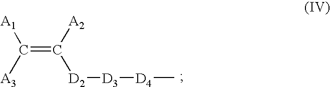

or R₃ is C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or R₃ is phenyl, naphthyl, C₂-C₁₈alkanoyl, benzoyl, C₁-C₁₈alkylsulfonyl, phenylsulfonyl, naphthylsulfonyl, anthryl sulfonyl or phenanthrylsulfonyl;
wherein the groups phenyl, naphthyl, C₂-C₁₈alkanoyl, benzoyl, C₁-C₁₈alkylsulfonyl, phenylsulfonyl, naphthylsulfonyl, anthrylsulfonyl and phenanthrylsulfonyl optionally are substituted by one or more Ar₂, OH, C₁-C₁₈alkyl, C₁-C₁₀haloalkyl, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy, benzoyloxy and/or a group of formula IV

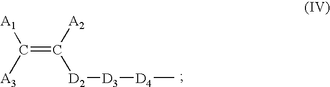

or R₃ is hydrogen;
R₄ and R₅ independently of each other are hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or R₄ and R₅ independently of each other are phenyl, naphthyl, C₂-C₁₈alkanoyl, benzoyl, C₁-C₁₈alkylsulfonyl, phenylsulfonyl, naphthylsulfonyl, anthrylsulfonyl or phenanthrylsulfonyl;
wherein the groups phenyl, naphthyl, C₂-C₁₈alkanoyl, benzoyl, C₁-C₁₈alkylsulfonyl, phenylsulfonyl, naphthylsulfonyl, anthrylsulfonyl and phenanthrylsulfonyl optionally are substituted by one or more Ar₂, OH, C₁-C₁₈alkyl, C₁-C₁₀haloalkyl, halogen, NO₂, CN, C₁-C₁₈alkoxy, phenoxy, C₁-C₁₈alkylamino, C₁-C₁₂alkylthio, C₁-C₁₈alkylsulfonyloxy, phenylsulfonyloxy, (4-methylphenyl)sulfonyloxy, C₂-C₁₈alkanoyloxy, and/or by benzoyloxy;
or R₄ and R₅, together with the nitrogen atom to which they are attached, form a 5-, 6- or 7-membered ring which optionally is interrupted by one or more O, NR₈ or CO;
R₆ is hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₈, CO, SO and/or SO₂, C₃-C₃₀cycloalkyl which is interrupted by one or more O, S, NR₈, CO, SO and/or SO₂, C₄-C₃₀cycloalkenyl which is interrupted by one or more O, S, NR₈, CO, SO and/or SO₂, C₂-C₁₂alkenyl which is interrupted by one or more O, S, NR₈, CO, SO and/or SO₂;
or R₆ is phenyl, naphthyl, C₂-C₁₈alkanoyl, benzoyl, C₁-C₁₈alkylsulfonyl, phenylsulfonyl, naphthylsulfonyl, anthrylsulfonyl or phenanthrylsulfonyl;
wherein the groups phenyl, naphthyl, C₂-C₁₈ alkanoyl, benzoyl, C₁-C₁₈alkylsulfonyl, phenylsulfonyl, naphthylsulfonyl, anthrylsulfonyl and phenanthrylsulfonyl optionally are substituted by one or more Ar₂, OH, C₁-C₁₈alkyl, C₁-C₁₀haloalkyl, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy and/or by benzoyloxy;
R₇ is hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or R₇ is phenyl, or naphthyl both of which optionally are substituted by one or more Ar₂, OH, C₁-C₁₈alkyl, C₁-C₁₀haloalkyl, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy and/or by benzoyloxy;
R₈ is C₃-C₃₀cycloalkyl, C₁-C₁₈alkyl, C₁-C₁₀haloalkyl, C₂-C₁₂alkenyl, C₄-C₃₀cycloalkenyl or phenyl-C₁-C₃alkyl;
Ar₂ is phenyl, biphenylyl or naphthyl,
wherein the groups phenyl, biphenylyl and naphthyl optionally are substituted by one or more OH, C₁-C₁₈alkyl, C₁-C₁₀haloalkyl, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy and/or by benzoyloxy;
A₁, A₂ and A₃ independently of each other are hydrogen, halogen, CN, C₁-C₁₈alkyl, C₁-C₁₈alkyl which is substituted by OR₃; or A₁, A₂ and A₃ independently of each other are C₁-C₁₀haloalkyl, (CO)R₂, (CO)OR₃, or (CO)NR₄R₅;
D₂ is a direct bond, O, (CO)O, (CO)S, (CO)NR₆, SO₂, OSO₂, Ar′₂, C₁-C₁₈alkylene; or A₃ and D₂ together with the ethylenically unsaturated double bond to which they are attached form C₃-C₃₀cycloalkenyl which optionally is interrupted by one or more O, S, N, NR₆, CO, SO and/or SO₂;
or A₂ and D₂ together with the carbon of the ethylenically unsaturated double bond to which they are attached form C₃-C₃₀cycloalkyl which optionally is interrupted by one or more O, S, N, NR₆, CO, SO and/or SO₂;
D₃ and D₄ independently of each other are a direct bond, O, S, NR₆, CO, O(CO), (CO)O, (CO)S, (CO)NR₆, SO, SO₂, OSO₂, Ar′₂,

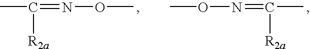

C₁-C₁₈alkylene, C₃-C₃₀cycloalkylene, C₂-C₁₂alkenylene, C₄-C₃₀cycloalkenylene, C₂-C₁₈alkylene which is interrupted by one or more O, S, NR₆, CO, SO and/or SO₂, C₃-C₃₀cycloalkylene which is interrupted by one or more O, S, NR₆, CO, SO and/or SO₂, C₄-C₃₀cycloalkenylene which is interrupted by one or more O, S, NR₆, CO, SO and/or SO₂, C₂-C₁₂alkenylene which is interrupted by one or more O, S, NR₆, CO, SO and/or SO₂;
wherein the groups C₁-C₁₈alkylene, C₃-C₃₀cycloalkylene, C₂-C₁₂alkenylene, C₄-C₃₀cycloalkenylene, interrupted C₂-C₁₈alkylene, interrupted C₃-C₃₀cycloalkylene interrupted C₄-C₃₀cycloalkenylene and interrupted C₂-C₁₂alkenylene optionally are substituted by one or more Ar₂, OH, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy, and/or by benzoyloxy;
wherein all radicals D₃ and D₄ optionally additionally are substituted by a group having a —O—C-bond or a —O—Si-bond which cleaves upon the action of an acid;
Ar′₂ is phenylene, biphenylene, naphthylene or heteroarylene;
wherein the groups phenylene, biphenylene, naphthylene and heteroarylene optionally are substituted by one or more OH, C₁-C₁₈alkyl, C₁-C₁₀haloalkyl, halogen, NO₂, CN, C₁-C₁₈alkoxy, phenoxy, NR₄R₆, C₁-C₁₂alkylthio, C₁-C₁₈alkylsulfonyloxy, phenylsulfonyloxy, (4-methylphenyl)sulfonyloxy, C₂-C₁₈alkanoyloxy and/or by benzoyloxy;
R₂ₐ has one of the meanings given for R₂;
provided that at least one of the radicals R₂, R₃, Ar₁ or Ar₁′ comprises a group of the formula IV.

The compounds of the formula I, II and III are characterized in that they contain a haloalkyl group or nitrile group adjacent to the oxime moiety, and that they have at least one polymerizable ethylenically unsaturated group at chromophor moiety, i.e. in R₂, Ar₁ and/or Ar′₁.

Of interest are in particular compounds of the formula I, II and III, wherein

R₁ is C₁-C₁₈alkylsulfonyl, C₁-C₁₀haloalkylsulfonyl;
or R₁ is phenylsulfonyl which optionally are substituted by one or more C₁-C₁₈alkyl, C₁-C₁₀haloalkyl, halogen or NO₂;
R′₁ is phenylenedisulfonyl or C₁-C₁₀haloalkylenedisulfonyl;
R₂ is CN or C₁-C₁₀haloalkyl;
Ar₁ is phenyl, fluorenyl, naphthyl or heteroaryl,
all of which optionally are substituted by one or more OR₃, NR₄R₅, SR₇, or a group of formula IV

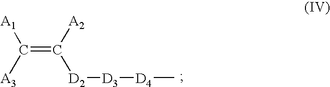

R₂ₐ has one of the meanings given for R₂;
Ar′₁ is phenylene or heteroarylene which optionally is substituted by a group of formula IV

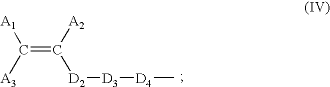

### or Ar′₁ is —Ar″₁—X₁—Y₁—X₁—Ar″₁—;

Ar″₁ is phenylene or naphthylene;

### X₁ is O, NR₆ or S;

V₁ is C₁-C₁₈alkylene which optionally is substituted by a group of formula IV

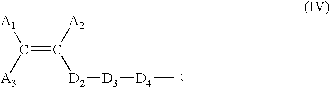

R₃ is C₁-C₁₈alkyl which optionally is substituted by a group of formula IV

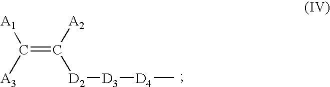

R₄ and R₅ independently of each other are hydrogen, C₁-C₁₈alkyl which optionally is substituted by a group of formula IV

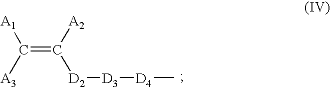

A₁, A₂ and A₃ independently of each other are hydrogen or C₁-C₁₈alkyl;
D₂ is (CO)O, Ar′₂, C₁-C₁₀alkylene;
or A₃ and D₂ together with the ethylenically unsaturated double bond to which they are attached form C₃-C₃₀cycloalkenyl which optionally is interrupted by one or more one or more N or CO;
or A₂ and D₂ together with the carbon of the ethylenically unsaturated double bond to which they are attached form C₃-C₃₀cycloalkyl which optionally is interrupted by one or more N or CO;
D₃ and D₄ independently of each other are a direct bond, O, S, CO, O(CO), (CO)O, Ar′₂,

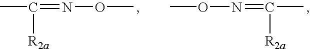

C₃-C₃₀cycloalkylene, C₁-C₁₈alkylene, C₂-C₁₈alkylene which is interrupted by one or more O, CO, NR₆ and/or SO₂;
R₆ is hydrogen; and
Ar′₂ is phenylene.

Especially interesting are compounds of the formula I, II and III described above, wherein

R₁ is C₁-C₁₀haloalkylsulfonyl; or R₁ is phenylsulfonyl which optionally is substituted by C₁-C₁₀haloalkyl or NO₂;
R₂ is C₁-C₁₀haloalkyl;
Ar₁ is phenyl fluorenyl, naphthyl or heteroaryl, all of which are substituted by a group of formula IV

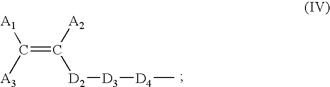

A₁, A₂ and A₃ independently of each other are hydrogen or C₁-C₄alkyl; and

### D₂ is (CO)O;

D₃ and D4 independently of each other are a direct bond, (CO)O, O,

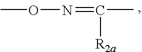

C₂-C₁₈alkylene or C₂-C₁₈alkylene which is interrupted by one or more CO or NR₆;

### R₂ₐ is CN; and

R₆ is hydrogen.

The compounds of the formula I, II and III can be polymerized, either with one another or with other components comprising ethylenically unsaturated polymerizable groups.

Subject of the invention therefore also is a polymer comprising at least one repeating unit derived from the compound of the formula I, II and/or III as described above.

Interesting polymers are such additionally to the at least one repeating unit derived from the compound of the formula I, II and/or III, comprising one or more identical or different repeating units derived from ethylenically unsaturated compounds selected from the group of formula V

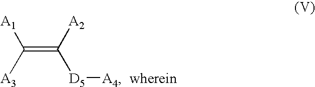

A₁, A₂ and A3 independently of each other are hydrogen, halogen, CN, C₁-C₁₈alkyl which is substituted by OR₃; or A₁, A₂ and A₃ independently of each other are C₁-C₁₀haloalkyl, (CO)R₇, (CO)OR₃, or (CO)NR₄R₅;
A₄ is C₁-C₁₈alkyl, C₂-C₁₈alkyl which is interrupted by one or more O, S, NR₆, CO, SO and/or SO₂,
C₃-C₃₀cycloalkyl, C₃-C₃₀cycloalkyl which is interrupted by one or more O, S, NR₆, CO, SO and/or SO₂,
C₂-C₁₂alkenyl, C₂-C₁₂alkenyl which is interrupted by one or more O, S, NR₆, CO, SO and/or SO₂,
C₄-C₃₀cycloalkenyl, C₄-C₃₀cycloalkenyl which is interrupted by one or more O, S, NR₆, CO, SO and/or SO₂,
wherein the groups C₁-C₁₈alkyl, interrupted C₂-C₁₈alkyl, C₃-C₃₀cycloalkyl, interrupted C₃-C₃₀cycloalkyl, C₂-C₁₂alkenyl, interrupted C₂-C₁₂alkenyl, C₄-C₃₀cycloalkenyl and interrupted C₄-C₃₀cycloalkenyl optionally are substituted by one or more Ar₂, OR₃, (CO)OR₃, O(CO)R₇, halogen, NO₂, CN, NR₄R₅ C₁-C₁₂alkylthio, C₁-C₁₈alkylsulfonyloxy, phenylsulfonyloxy, and/or (4-methylphenyl)sulfonyloxy;
or A₄ is hydrogen, halogen, NO₂, CN, Ar₂, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR_{T}, SO₂R₇ and/or OSO₂R₇;
D₅ is a direct bond, O, CO, (CO)O, (CO)S, (CO)NR₆, SO₂, or OSO₂;
or D₅ is C₁-C₁₈alkylene;
or D₅ is a group Ar′₂;
optionally the radicals A₃ and D₅, together with the ethylenically unsaturated double bond to which they are attached form C₃-C₃₀cycloalkenyl which optionally is interrupted by one or more O, S, NR₆, CO, SO and/or SO₂;
or optionally the radicals A₂ and D₅ together with the carbon atom of the ethylenically unsaturated double bond to which they are attached form C₃-C₃₀cycloalkyl which optionally is interrupted by one or more O, S, N, NR₆, CO, SO and/or SO₂; and
R₃, R₄, R₅, R₆, R₇, Ar′₂ and Ar₂ are as defined above.

A further subject of the invention is a polymer, wherein

A₁, A₂ and A₃ independently of each other are hydrogen or C₁-C₁₈alkyl;
A₄ is hydrogen, C₃-C₃₀cycloalkyl, C₃-C₃₀cycloalkyl which is interrupted by one or more O and/or CO, C₁-C₁₈alkyl, C₂-C₁₈alkyl which is interrupted by one or more O and/or CO, C₄-C₃₀cycloalkenyl which is interrupted by one or more O and/or CO;
wherein the groups C₃-C₃₀cycloalkyl, interrupted C₃-C₃₀cycloalkyl, C₁-C₁₈alkyl, interrupted C₁-C₁₈alkyl, and interrupted C₄-C₃₀cycloalkenyl optionally are substituted by one or more OR₃, (CO)OR₃ or O(CO)R₇;

### D₅ is (CO)O;

R₃ is C₁-C₁₈alkyl, C₂-C₁₈alkyl which is interrupted by one or more O and/or CO, C₃-C₃₀cycloalkyl, C₃-C₃₀cycloalkyl which is interrupted by one or more O and/or CO, or is C₄-C₃₀cycloalkenyl which is interrupted by one or more O and/or CO; or R₃ is hydrogen;
R₇ is C₃-C₃₀cycloalkyl, C₂-C₁₈alkyl which is interrupted by one or more O and/or CO, C₃-C₃₀cycloalkyl which is interrupted by one or more O and/or CO, C₄-C₃₀cycloalkenyl which is interrupted by one or more O and/or CO,
or R₇ is hydrogen.

In particular interesting are the compounds as given in the examples 1-11 and 12-31, as well as the compounds the following formulae (a)-(s):

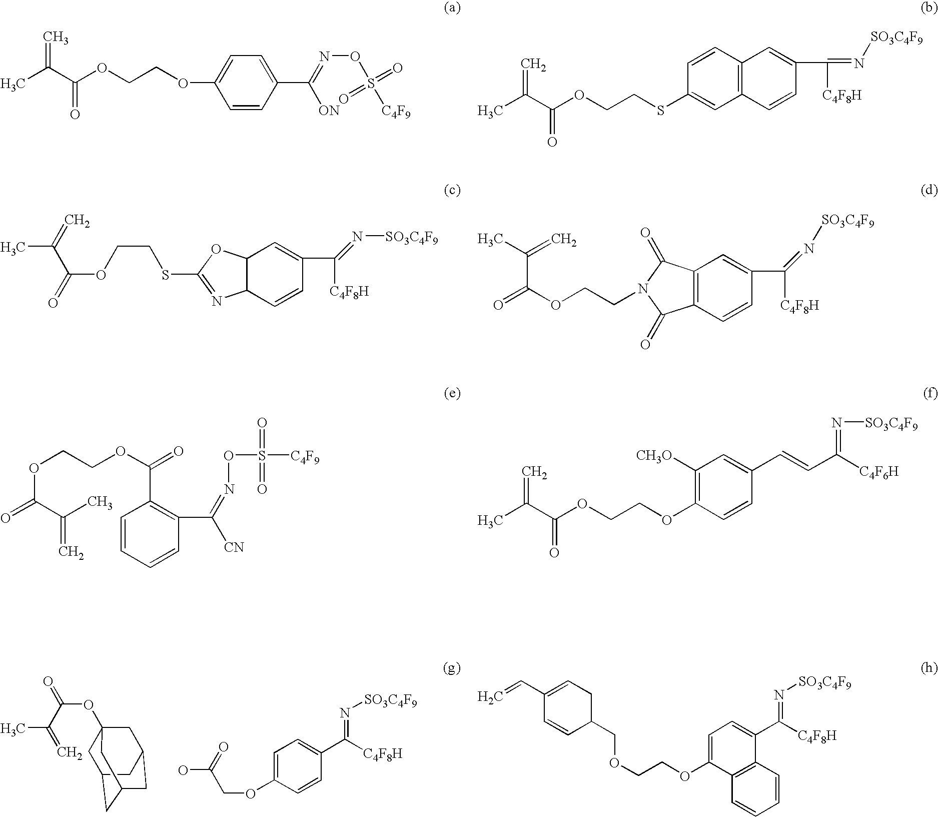

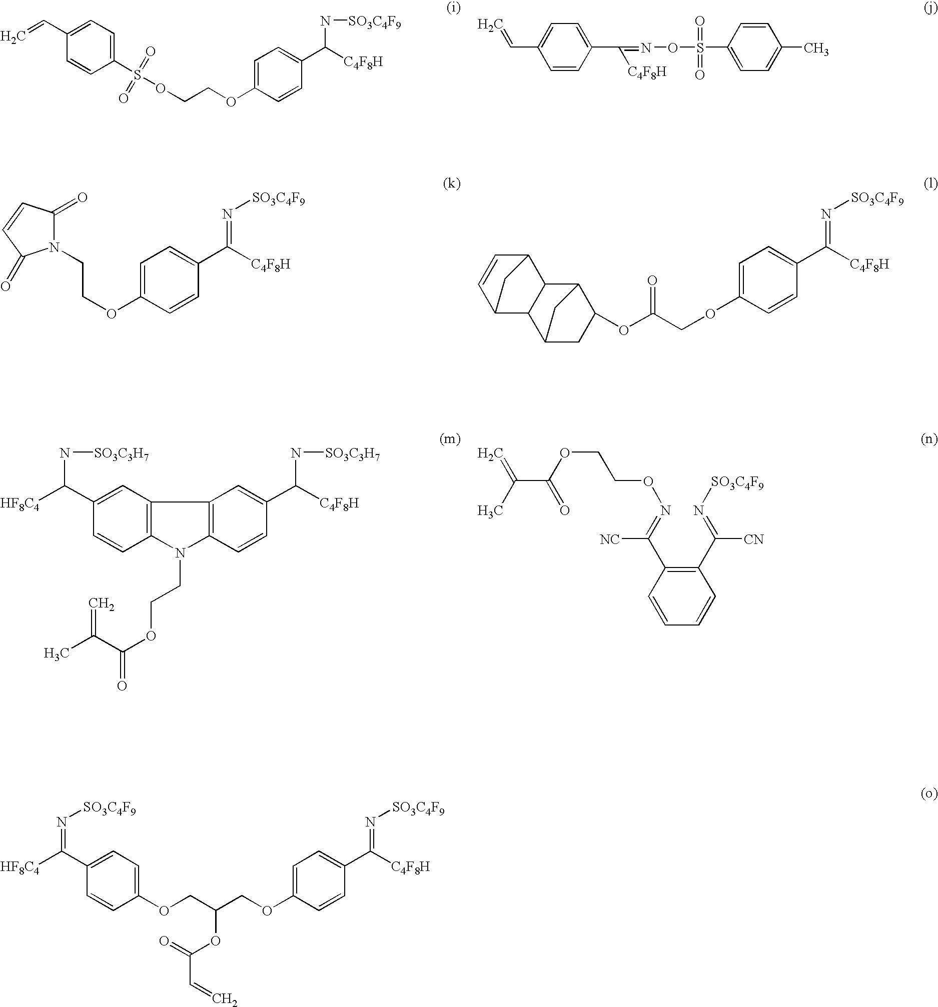

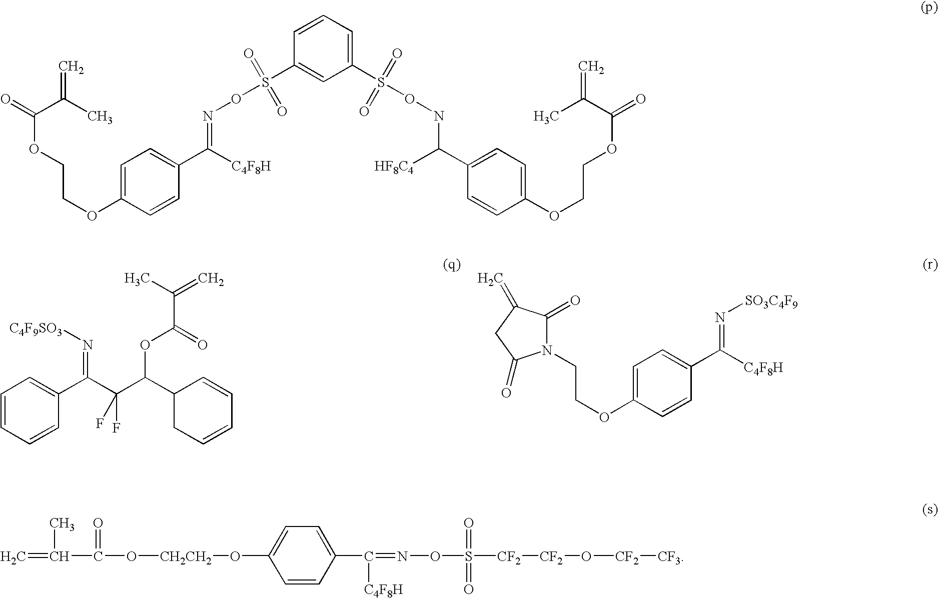

C₁-C₁₈alkyl is linear or branched and is, for example, C₁-C₁₆-, C₁-C₁₂-, C₁-C₈-, C₁-C₆- or C₁-C₄-alkyl. Examples are methyl, ethyl, propyl, isopropyl, n-butyl, sec-butyl, isobutyl, tert-butyl, pentyl, hexyl, heptyl, 2,4,4-trimethylpentyl, 2-ethylhexyl, octyl, nonyl, decyl, undecyl, dodecyl, tetradecyl, pentadecyl, hexadecyl, heptadecyl and octadecyl, preferably C₁-C₄alkyl, such as methyl, isopropyl or butyl.

C₂-C₁₈alkyl, which is interrupted by one or more O, S, NR₆ and/or CO, or NR₈ is interrupted, for example, from one to five times, for example from one to three times or once or twice, by non-successive O, S, NR₆ and/or CO or NR₈. Accordingly, resulting structural units are for example: O(CH₂)₂OH, O(CH₂)₂OCH₃, O(CH₂CH₂O)₂CH₂CH₃, CH₂—O—CH₃, CH₂CH₂—O—CH₂CH₃, [CH₂CH₂O]_{y}—CH₃, wherein y=1-5, (CH₂CH₂O)₅CH₂CH₃, CH₂—CH(CH₃)—O—CH₂—CH₂CH₃, CH₂—CH(CH₃)—O—CH₂—CH₃, S(CH₂)₂SCH₃, (CH₂)₂NHCH₃, (CH₂)₂O(CO)CH₃, (CH₂)₂(CO)OCH₃ or (CH₂)₂NH(CO)CH₃.

If, in the context of the present invention a group, e.g. alkyl or alkylene, is interrupted by one or more defined radicals, e.g. O, S, NR₆, SO₂, SO₂O and/or CO, the “interrupting” radicals not only are meant to be situated in between the interrupted group, for example the alkyl or alkylene, but also are meant to be terminal.

C₃-C₃₀cycloalkyl is a mono- or polycyclic aliphatic ring, for example a mono-, bi- or tricyclic aliphatic ring, e.g. C₃-C₂₀-, C₃-C₁₈-, C₃-C₁₂-, C₃-C₁₀cycloalkyl. C₃-C₃₀cycloalkyl in the context of the present application is to be understood as alkyl which at least comprises one ring, i.e. also carbocyclic aliphatic rings, which are substituted by alkyl are covered by this definition. Examples of monocyclic rings are cyclopropyl, cyclobutyl, cyclopentyl, cyclohexyl, or cycloheptyl, especially cyclopentyl and cyclohexyl. Further examples are structures like

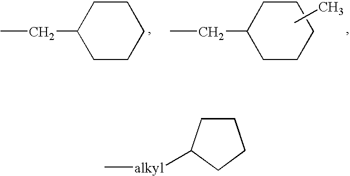

e.g.

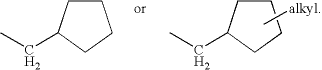

Examples of polycyclic rings are perhydroanthracyl, perhydrophenyathryl, perhydronaphthyl, perhydrofluorenyl, perhydrochrysenyl, perhydropicenyl, adamantyl, bicyclo[1.1.1]pentyl, bicyclo[4.2.2]decyl, bicyclo[2.2.2]octyl, bicyclo[3.3.2]decyl, bicyclo[4.3.2]undecyl, bicyclo[4.3.3]dodecyl, bicyclo[3.3.3]undecyl, bicyclo[4.3.1]decyl, bicyclo[4.2.1]nonyl, bicyclo[3.3.1]nonyl, bicyclo[3.2.1]octyl,

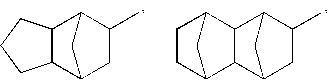

and the like. Also alkyl-substituted polycyclic and bridged rings are meant to be covered by the definition “cycloalkyl” in the context of the present invention, e.g. number

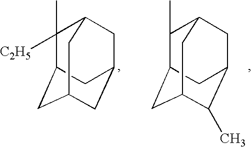

etc.

Also “spiro”-cycloalkyl compounds are covered by the definition C₃-C₃₀cycloalkyl in the present context, e.g. spiro[5.2]octyl, spiro[5.4]decyl, spiro[5.5]undecyl. More examples of polycyclic cycloalkyl groups, which are subject of the respective definition in the compounds of the present invention are listed in EP 878738, page 11 and 12, wherein to the formulae (1)-(46) a bond to achieve the “yl” has to be added. The person skilled in the art is aware of this fact.

In general, the cycloaliphatic rings may form repeating structural units.

C₃-C₃₀cycloalkyl which is interrupted by one or more O, S, NR₆ and/or CO is a mono- or polycyclic aliphatic ring which is interrupted by one or more O, S, NR₆ and/or CO, for example,

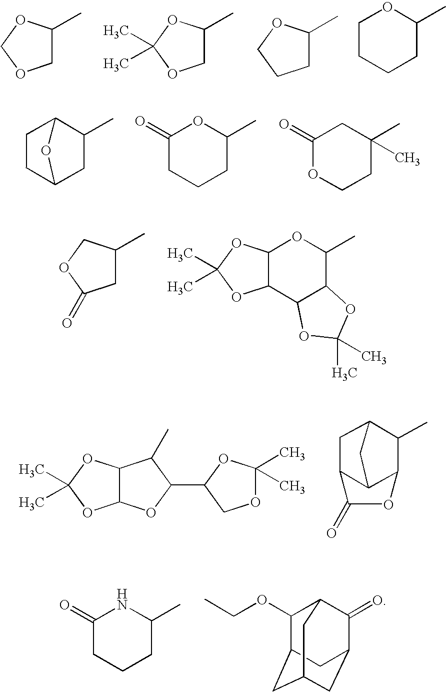

C₂-C₁₂alkenyl radicals are for example mono- or polyunsaturated, linear or branched and are for example C₂-C₈-, C₂-C₆- or C₂-C₄alkenyl. Examples are allyl, methallyl, vinyl, 1,1-dimethylallyl, 1-butenyl, 3-butenyl, 2-butenyl, 1,3-pentadienyl, 5-hexenyl or 7-octenyl, especially allyl or vinyl.

C₃-C₃₀cycloalkenyl is a mono- or polycyclic and mono- or polyunsaturated ring, for example a mono-, bi-, tri- or tetracyclic mono- or polyunsaturated ring, e.g. C₄-C₂₀-, C₄-C₁₈-, C₄-C₁₂-, C₄-C₁₀cycloalkenyl. Examples of cycloalkenyl are cyclobutenyl, cyclopentenyl, cyclohexenyl, cycloheptenyl. Also bridged alkenyl groups are covered by the above definition, for example

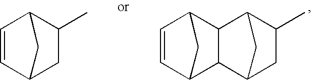

etc., especially cyclopentenyl, cyclohexenyl,

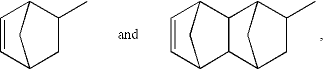

C₃-C₃₀cycloalkenyl which is interrupted by one or more O, S, NR₆ and/or CO is a mono- or polycyclic and mono- or polyunsaturated ring, which is interrupted by one or more O, S, NR₆ and/or CO, for example,

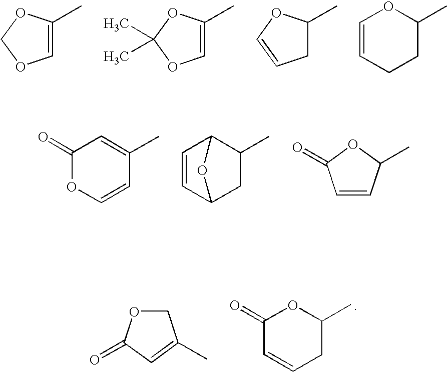

C₁-C₁₈alkylene is linear or branched alkylene. Examples are ethylene, propylene, butylene, pentylene, hexylene.

C₂-C₁₈alkylene, which is interrupted by one or more O, S, NR₆, O(CO), S(CO), NR₆(CO), SO, SO₂ and/or OSO₂, is interrupted, for example, from one to five times, for example from one to three times or once or twice, by “non-successive O”, by S, NR₆, O(CO), S(CO), NR₆(CO), SO, SO₂ and/or OSO₂. “Interrupted” in this definition in the context of the present application is also meant to comprise C₂-C₁₈alkylene having one or more of said defined groups attached at one end or both ends of the alkyl chain. Accordingly, resulting structural units are for example: —O(CH₂)₂—, —O(CH₂)₂OCH₂—, —O(CH₂CH₂O)₂— —S(CH₂)₂— —(CH₂)₂NH—, —(CH₂)₂O(CO)CH₂—, —(CH₂)₂O(SO₂)CH₂—, —CH₂CH₂NHCO—.

C₃-C₃₀cycloalkylene is a mono- or polycyclic aliphatic ring, for example a mono-, bi- or tricyclic aliphatic ring, e.g. C₃-C₂₀-, C₃-C₁₈-, C₃-C₁₂-, C₃-C₁₀cycloalkylene. Examples of monocyclic rings are cyclopropylene, cyclobutylene, cyclopentylene, cyclohexylene, or cycloheptylene. Examples of polycyclic rings are perhydroanthracylene, perhydrophenyathrylene, perhydronaphthylene, perhydrofluorenylene, perhydrochrysenylene, perhydropicenylene, adamantylene, bicyclo[1.1.1]pentylene, bicyclo[4.2.2]decylene, bicyclo[2.2.2]octylene, bicyclo[3.3.2]decylene, bicyclo[4.3.2]undecylene, bicyclo[4.3.3]dodecylene, bicyclo[3.3.3]undecylene, bicyclo[4.3.1]decylene, bicyclo[4.2.1]nonylene, bicyclo[3.3.1]nonylene, bicyclo[3.2.1]octylene,

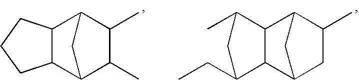

and the like. Also “spiro”-cycloalkylene compounds are covered by the definition C₃-C₃₀cycloalkylene in the present context, e.g. spiro[5.2]octylene, spiro[5.4]decylene, spiro[5.5]undecylene. More examples of polycyclic cycloalkylene groups, which are subject of the respective definition in the compounds of the present invention are listed in EP 878738, page 11 and 12, wherein to the formulae (1)-(46) two bonds to achieve the “ylene” has to be added. The person skilled in the art is aware of this fact.

C₃-C₃₀cycloalkylene which is interrupted by one or more O, S, NR₆, O(CO), NR₆CO, is a mono- or polycyclic aliphatic ring which is interrupted by one or more O, S, NR₆, O(CO), SCO, NR₆CO, for example,

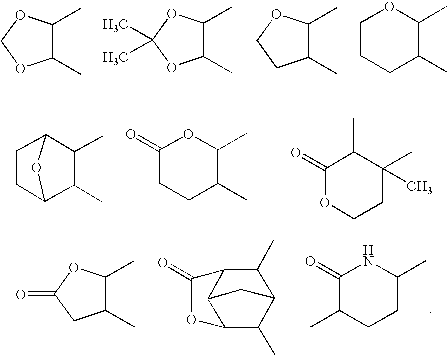

C₂-C₁₂alkenylene radicals are for example mono- or polyunsaturated, linear or branched and are for example C₂-C₈-, C₂-C₆- or C₂-C₄alkenylene. Examples are —CH═CHCH₂—, —CH═C(CH₃)CH₂—, —CH═C(CH₃)—,

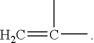

C₄-C₃₀cycloalkenylene is a mono- or polycyclic and mono- or polyunsaturated ring, for example a mono-, bi-, tri- or tetracyclic mono- or polyunsaturated ring, e.g. C₄-C₂₀ -, C₄-C₁₈-, C₄-C₁₂-, C₄-C₁₀cycloalkenylene. Examples are

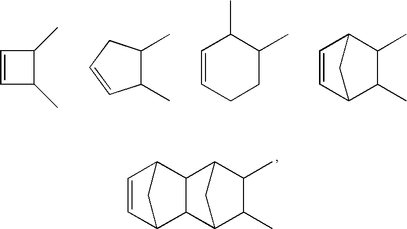

etc.

C₄-C₃₀cycloalkenylene which is interrupted by one or more O, S, NR₆, O(CO) and/or NR₆(CO) is a mono- or polycyclic and mono- or polyunsaturated ring, which is interrupted by one or more O, S, NR₆, O(CO), SCO, NR₆CO, for example

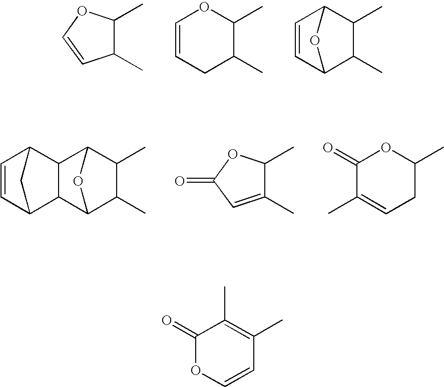

etc.

Substituted phenyl carries from one to five, for example one, two or three, especially one or two, substituents on the phenyl ring. The substitution is preferably in the 4-, 3,4-, 3,5- or 3,4,5-position of the phenyl ring.

When the radicals phenyl, biphenyl, naphthyl, fluorenyl, phenanthryl, anthracyl and heteroaryl are substituted by one or more radicals, they are, for example, mono- to penta-substituted, for example mono-, di- or tri-substituted, especially mono- or di-substituted.

When Ar₁ is phenyl, biphenyl, fluorenyl, naphthyl, anthracyl, phenanthryl, or heteroaryl substituted by one or more C₁-C₁₈alkyl, C₂-C₁₂alkenyl, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇, and the substituents C₁-C₁₈alkyl, C₂-C₁₂alkenyl, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇ form 5-, 6- or 7-membered rings, via the radicals C₁-C₁₈alkyl, C₂-C₁₂alkenyl, R₃, R₄, R₅, R₆ and/or R₇, with further substituents on the phenyl, biphenyl, naphthyl, anthracyl, phenanthryl, or heteroaryl ring or with one of the carbon atoms of the phenyl, biphenyl, naphthyl, anthracyl, phenanthryl, or heteroaryl ring, for example the following structural units are obtained

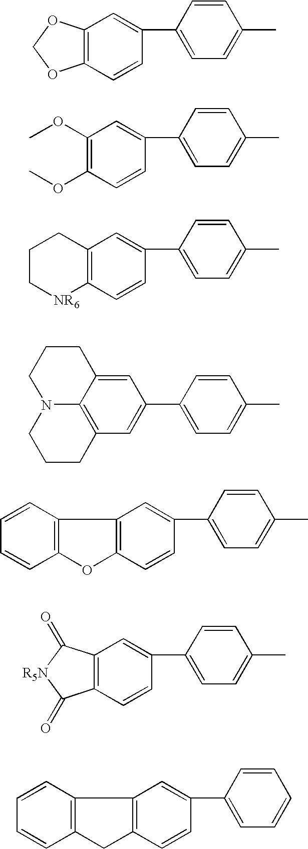

etc.

If in Ar₁ the substituents C₁-C₁₈alkyl form alkylene bridges from one carbon atom of the biphenyl, naphthyl, or fluorenyl ring to another carbon atom of said ring, in particular ethylene, propylene and butylene bridges are formed and for example the following structures are obtained

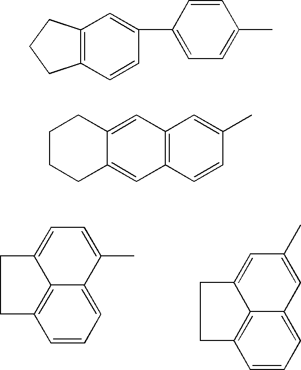

etc. The definition according to the present application in this connection also is intended to cover branched alkylene bridges:

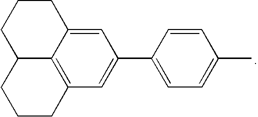

In case said alkylene bridges are condensed with further phenyl rings for example the following structure is given

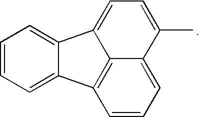

When Ar′₁ is phenylene, biphenylene, naphthylene,

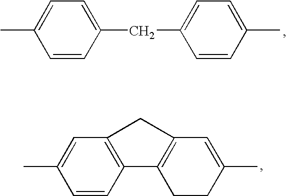

heteroarylene, oxydiphenylene or

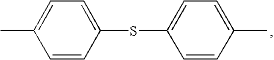

all of which are substituted by one or more C₁-C₁₈alkyl, C₂-C₁₂alkenyl, (CO)R₂, (CO)OR₃, (CO)NR₄R₅, O(CO)R₂, O(CO)OR₃, O(CO)NR₄R₅, NR₆(CO)R₇, NR₆(CO)OR₃, OR₃, NR₄R₅, SR₆, SOR_{T}, SO₂R₇ and/or OSO₂R₂, and the substituents C₁-C₁₈alkyl, C₂-C₁₂alkenyl, (CO)R₂, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR_{T}, SO₂R₂ and/or OSO₂R₇ form 5-, 6- or 7-membered rings, via the radicals C₁-C₁₈alkyl, C₂-C₁₂alkenyl, R₃, R₄, R₅, R₆ and/or R₇, with further substituents on the phenylene, biphenylene, naphthylene,

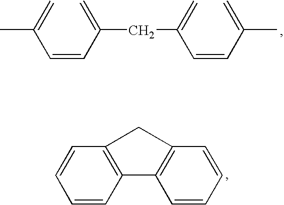

heteroarylene, oxydiphenylene or

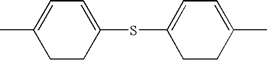

ring or with one of the carbon atoms of the phenylene, biphenylene, naphthylene,

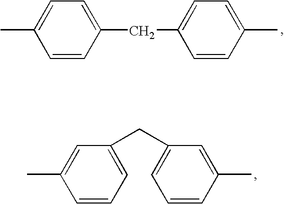

heteroarylene,

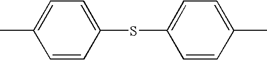

or oxydiphenylene ring, for example the following structural units are obtained

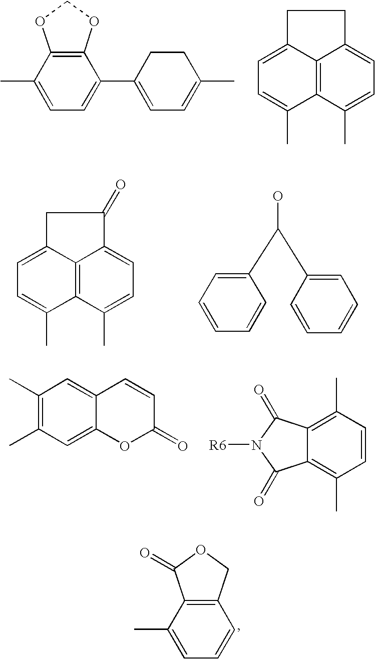

etc.

Camphoryl, 10-camphoryl, are camphor-10-yl, namely

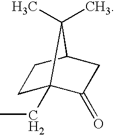

C₂-C₁₈alkanoyl is e.g. C₂-C₁₂, C₂-C₈-, C₂-C₆- or C₂-C₄alkanoyl, wherein the alkyl moiety is linear or branched. Examples are acetyl, propionyl, butanoyl or hexanoyl, especially acetyl.

C₁-C₁₈alkoxy is e.g. C₁-C₁₂-, C₁-C₈-, C₁-C₆-, C₁-C₄alkoxy, and is linear or branched. Examples are methoxy, ethoxy, propoxy, n-butoxy, t-butoxy, octyloxy and dodecyloxy.

In C₁-C₁₂alkylthio the alkyl moiety is for example linear or branched. Examples are methylthio, ethylthio, propylthio or butylhtio.

C₂-C₁₈alkoxycarbonyl is (C₁-C₁₇alkyl)-O—C(O)—, wherein C₁-C₁₇alkyl is linear or branched and is as defined above up to the appropriate number of carbon atoms. Examples are C₂-C₁₀-, C₂-C₈-, C₂-C₆- or C₂-C₄alkoxycarbonyl, such as methoxycarbonyl, ethoxycarbonyl, propoxycarbonyl, butoxycarbonyl or pentoxycarbonyl.

C₁-C₁₀haloalkyl are for example C₁-C₈-, C₁-C₆- or C₁-C₄-alkyl mono- or poly-substituted by halogen, the alkyl moieties being, for example, as defined above. There are, for example, from 1 to 23 halogen substituents at the alkyl radical. Examples are chloromethyl, trichloromethyl, trifluoromethyl, nonafluorobutyl or 2-bromopropyl, especially trifluoromethyl or trichloromethyl. Preferred is C₁-C₁₀-fluoroalkyl.

C₁-C₁₀haloalkylene is linear or branched alkylene mono- or poly-substituted by halogen, the alkylene moieties being, for example, as defined above. Examples are tetrafluoroethylene, hexafluoropropylene, dibromomethylene.

C₂-C₁₀haloalkanoyl is (C₁-C₉haloalkyl)-C(O)—, wherein C₁-C₉haloalkyl is as defined above up to the appropriate number of carbon atoms. Examples are chloroacetyl, trichloroacetyl, trifluoroacetyl, pentafluoropropionyl, perfluorooctanoyl, or 2-bromopropionyl, especially trifluoroacetyl or trichloroacetyl.

Halobenzoyl is benzoyl which is mono- or poly-substituted by halogen and/or C₁-C₄haloalkyl, C₁-C₄-haloalkyl being as defined above. Examples are pentafluorobenzoyl, trichlorobenzoyl, trifluoromethylbenzoyl, especially pentafluorobenzoyl.

Halogen is fluorine, chlorine, bromine or iodine, especially chlorine or fluorine, preferably fluorine.

Phenyl-C₁-C₃alkyl is, for example, benzyl, 2-phenylethyl, 3-phenylpropyl, α-methylbenzyl or α,α-dimethylbenzyl, especially benzyl.

If R₄ and R₅ together with the nitrogen atom to which they are bonded form a 5-, 6- or 7-membered ring that optionally is interrupted by O, NR₆ or CO, for example the following structures are obtained

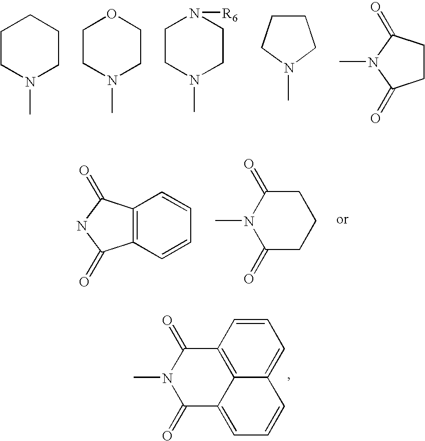

etc.

The definitions C₁-C₁₈alkylsulfonyl, camphorylsulfonyl, phenyl-C₁-C₃alkylsulfonyl, and C₃-C₃₀cycloalkylsulfonyl refer to the corresponding radicals C₁-C₁₈alkyl, camphoryl, phenyl-C₁-C₃alkyl, and C₃-C₃₀cycloalkyl, as described in detail above, being linked to a sulfonyl group (—SO₂—). Accordingly, also phenylsulfonyl, naphthylsulfonyl, anthranylsulfonyl and phenanthrylsulfonyl refer to the corresponding radicals linked to a sulfonyl group.

C₂-C₁₈alkanoyloxy is (C₁-C₁₇alkyl)-C(O)—O—, wherein C₁-C₁₇alkyl is linear or branched and is as defined above up to the appropriate number of carbon atoms. Examples are C₂-C₁₀-, C₂-C₈-, C₂-C₆- or C₂-C₄alkanoyloxy, such as acetyloxy, ethanoyloxy, propanoyloxy, butanoyloxy or hexanoyloxy.

C₁-C₁₈alkylsulfonyloxy is (C₁-C₁₈alkyl)-S(O)₂—O—, wherein C₁-C₁₈alkyl is linear or branched and is as defined above up to the appropriate number of carbon atoms. Examples are C₁-C₁₀-, C₁-C₈-, C₁-C₆- or C₁-C₄alkylsulfonyloxy, such as methanesulfonyloxy, propanesulfonyloxy or hexanesulfonyloxy.

Accordingly, also phenylsulfonyloxy and (4-methylphenyl)sulfonyloxy refer to the corresponding radicals linked to a —S(O)₂—O— group.

In the present application, the term “heteroaryl” denotes unsubstituted and substituted radicals, for example 3-thienyl, 2-thienyl,

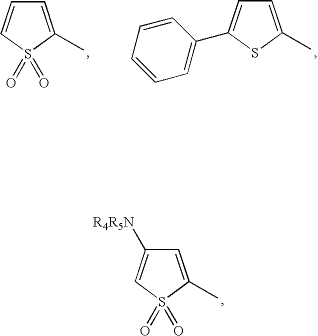

wherein R₄ and R₅ are as defined above, thianthrenyl, isobenzofuranyl, xanthenyl, phenoxanthiinyl,

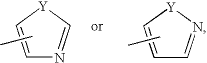

wherein Y is S, O or NR₆ and R₆ is as defined above. Examples thereof are pyrazolyl, thiazolyl, oxazolyl, isothiazolyl or isoxazolyl. Also included are, for example, furyl, pyrrolyl, 1,2,4-triazolyl,

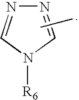

or 5-membered ring heterocycles having a fused-on aromatic group, for example benzimidazolyl, benzothienyl, benzofuranyl, benzoxazolyl and benzothiazolyl.

Other examples of “heteroaryls” are pyridyl, especially 3-pyridyl,

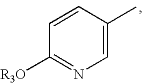

wherein R₃ is as defined above, pyrimidinyl, pyrazinyl, 1,3,5-triazinyl, 2,4-, 2,2- or 2,3-diazinyl, indolizinyl, isoindolyl, indolyl, indazolyl, purinyl, isoquinolyl, quinolyl, phenoxazinyl or phenazinyl. In this Application, the term “heteroaryl” also denotes the radicals thioxanthyl, xanthyl,

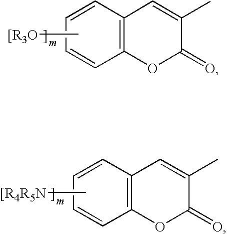

wherein m is O or 1 and R₃, R₄, R₅ are as defined above,

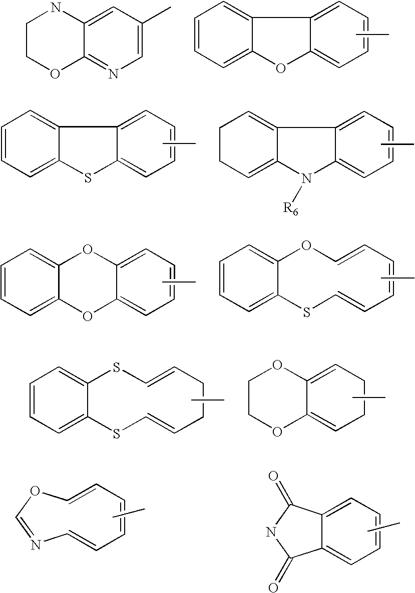

anthraquinonyl. Each of the heteroaryls may carry the substituents indicated above or in claim 1.

Phenylene is

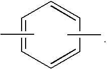

### Naphthylene is

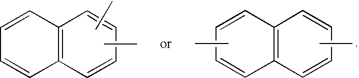

### Diphenylene is

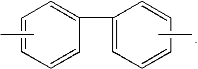

### Oxydiphenylene is

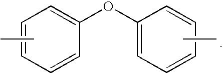

Heteroarylene is a divalent radical of the heteroaryl rings as described above, for example

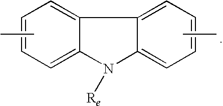

If the radicals A₃ and D₂ together with the ethylenically unsaturated double bond to which they are attached form C₃-C₃₀cycloalkenyl which optionally is interrupted by one or more O, S, N, NR₆ or CO for example the following structures are obtained

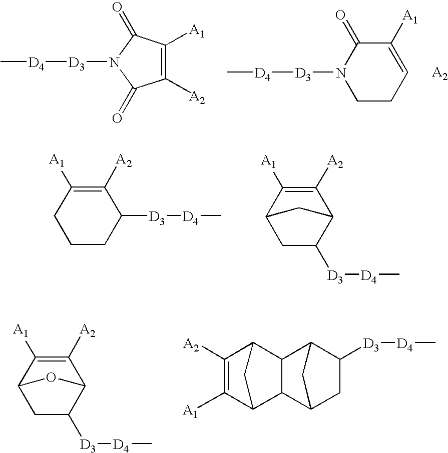

etc.

Groups having a —O—C-bond or a —O—Si-bond which cleaves upon the action of an acid, and being substituents of the radicals Ar₁ and Ar′₁ are acid cleavable groups which increase the solubility of the compounds of formula I, II or III in the alkaline developer after reaction with an acid. This effect is for example described in U.S. Pat. No. 4,883,740.

Examples of groups suitable as such substitutents are for example known orthoesters, trityl and benzyl groups, tert.-butyl esters of carboxylic acids, tert.-butyl carbonates of phenols or silyl ethers of phenols, e.g. —OSi(CH₃)₃, —CH₂(CO)OC(CH₃)₃, —(CO)OC(CH₃)₃, —O(CO)OC(CH₃)₃ or

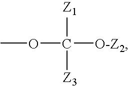

wherein Z₁ and Z₂ independently of one another are hydrogen, C₁-C₅alkyl, C₃-C₈-cycloalkyl, phenyl-C₁-C₃-alkyl, or Z₁ and Z₂ together are C₂-C₅alkylene, and

Z₃ is unsubstituted or halogen-substituted C₁-C₅alkyl, unsubstituted or halogen-substituted C₃-C₈cycloalkyl, or phenyl-C₁-C₃-alkyl, or, if Z₁ and Z₂ together are no C₂-C₅alkylene, Z₃ and Z₂ together may be C₂-C₅alkylene, which may be interrupted by an —O-atom or an —S-atom.

The terms “and/or” or “or/and” in the claims and throughout the specification are meant to express that not only one of the defined alternatives (substituents) may be present, but also several of the defined alternatives (substituents) together, namely mixtures of different alternatives (substituents).

The term “optionally substituted” means unsubstituted or substituted.

The term “at least” is meant to define one or more than one, for example one or two or three, preferably one or two.

Oxime sulfonates of formulae I, II and III can generally be prepared by methods described in the literature, for example by reacting suitable free oximes (R₁═H) of formula I″ or II″ with the desired (for example, sulfonic) acid halides or acid anhydrides of formula V or VI (for example, R₁Cl, R₁F or R₁—O—R₁).

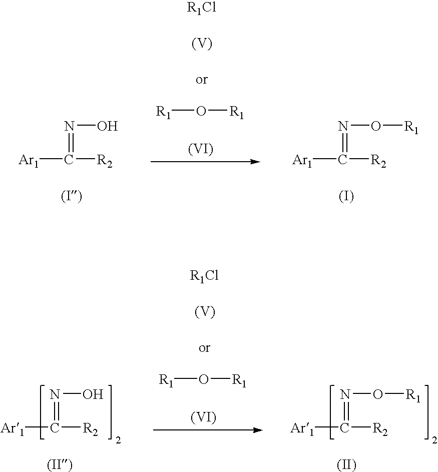

R₁, R₂, Ar₁ and Ar₁′ are defined as described above.

These reactions usually are carried out in an inert solvent such as for example toluene, methylene chloride, tetrahydrofuran (THF) or dimethylformamide (DMF) in the presence of a base, for example pyridine, a tertiary amine, such as triethylamine, or by reaction of the salt of an oxime with the desired acid chloride. These methods are disclosed, for example, in EP 48615. The sodium salts of oximes can be obtained, for example, by reacting the oxime in question with a sodium alcoholate in dimethylformamide. Such reactions are well known to those skilled in the art, and are generally carried out at temperatures in the range of −15 to +50° C., preferably 0 to 20° C.

The oximes required as starting materials can be obtained by a variety of methods described in standard chemistry textbooks (for instance in J. March, Advanced Organic Chemistry, 4th Edition, Wiley Interscience, 1992), or in specialized monographs, for example, S. R. Sandler & W. Karo, Organic functional group preparations, Vol. 3, Academic Press.

One of the most convenient methods is, for example, the reaction of ketones with hydroxyamine or its salt in polar solvents like ethanol or aqueous ethanol. In that case, a base such as sodium acetate is added to control the pH of the reaction mixture. It is well known that the rate of the reaction is pH-dependent, and the base can be added at the beginning or continuously during the reaction. Basic solvents such as pyridine can also be used as base and/or solvent or cosolvent. The reaction temperature is generally the refluxing temperature of the mixture, usually about 60-120° C.

Another convenient synthesis of oximes is the nitrosation of “active” methylene groups with nitrous acid or an alkyl nitrite. Both alkaline conditions, as described for example in Organic Syntheses coll. Vol. VI (J. Wiley & Sons, New York, 1988), pp 199 and 840, and acidic conditions, as described, for example, in Organic Synthesis coll. vol V, pp 32 and 373, coll. vol. III, pp 191 and 513, coll. vol. 11, pp. 202, 204 and 363, are suitable for the preparation of the oximes used as starting materials for the compounds according to the invention. Nitrous acid is usually generated from sodium nitrite. The alkyl nitrite can for example be methyl nitrite, ethyl nitrite, isopropyl nitrite, butyl nitrite, isoamyl nitrite.

The described syntheses can result in the formation of isomeric forms of the compounds of formula I, II and III. The double bond of the oximino group can exist in both the syn (cis, Z) and the anti (trans, E) form or as mixtures of the two geometrical isomers. In the present invention, both the individual geometrical isomers and any mixtures of two geometrical isomers can be used. The invention accordingly also relates to mixtures of isomeric forms of the compounds of formula I, II and III.

The compounds of formula I, II and III of the individual geometrical isomers (Z and E forms) and any mixtures of two geometrical isomers can be used, however, it has been found that the compounds of formula I, II and III of a specific configuration (tentatively assigned as E-form) are more thermally stable than the compounds of other configuration (tentatively assigned as Z-form). Therefore, preferred use of the compounds of the present invention are of formula formula I, II and III of the single more thermally stable isomer (tentatively assigned as E-form).

The syntheses of the oximes required as starting materials can result in the formation of a mixture of isomeric forms. Surprisingly, it has been found that the mixture of isomeric forms of the oximes required as starting materials is converted to a single isomeric form (tentatively assigned as E-form) by treatment with acid. Using these oximes of the single isomer (E-form) as the starting materials, the compounds of formula I, II and III of the thermally more stable single isomer are obtained. Accordingly the present invention also relates to a process for the synthesis of the thermally more stable isomer of the compounds of formula I, II and III by 1) conversion of the corresponding isomeric mixture of oximes to the oximes of the single isomeric form by treatment with an acid, and 2) reaction of the oximes of the single isomeric form with the desired acid halides or acid anhydride.

The conversion reactions of the isomeric mixture of oximes to the desired single isomer are usually carried out in an inert solvent such as methylene chloride, ethyl acetate, toluene, tetrahydrofuran, dimethylformamide or acetic anhydride in the presence of an acid such as hydrochloric acid, sulfuric acid, acetic acid, nitric acid, trifluoroacetic acid, or trifluoromethanesulfonic acid. The conversion reactions can also be carried out in acid solvent, e.g., formic acid, acetic acid optionally in the presence of other acid such as hydrochloric acid, sulfuric acid, nitric acid, trifluoroacetic acid, or trifluoromethanesulfonic acid. Such reactions are usually carried out at temperature in the range of −15° C. to +120° C., preferably 0° C. to 80° C., more preferably 5° C. to 40° C. The compounds are isolated by methods known to the person skilled in the art, e.g. distillation, recrystallisation, chromatographic methods.

Examples for conventional methods to obtain the oxime compounds of formula I′ and II′ as starting materials are given above.

Oxime sulfonates of the formulae I, II and III have at least one polymerizable ethylenically unsaturated double bond group of the formula IV. Hence polymers can be prepared employing the oxime sulfonates of the formulae I, II and/or III by methods described in the literature, for example by free radical polymerization, anionic polymerization, cationic polymerization, controlled free radical polymerization and so on.

The free radical polymerizations usually are carried out in an inert solvent such as for example water, methanol, 2-propanol, 1,4-dioxane, acetone, methyl isobutyl ketone, toluene, tetrahydrofuran (THF), propylene glycol monomethyl ether acetate (PGMEA), propylene glycol monomethyl ether (PGME), ethyl lactate (EL), or without solvent under an oxygen-free atmosphere. Peroxides such as for example dibenzoyl peroxide, diacetyl peroxide, di-t-butyl peroxalate and dicumyl peroxide; azocompounds such as for example azobis(isobutyronitrile) (AIBN), 1,1′-azobis(1-cyclohexanenitrile), 2,2-azobis(2-amidinopropane)dihydrochloride, dimethyl 2,2′-azobis(isobutyrate) and 2,2′-azobis[2-methyl-N-(2-hydroxyethyl)propionamide]; and redox systems such as for example Fe²⁺/H₂O₂ and dibenzoyl peroxide/dimethylaniline, are used as the initiator for free radical polymerization. Such reactions are well known to those skilled in the art, and generally carried out at temperature in the range of −10° C. to 150° C., preferably 40° C. to 120° C. Furthermore, for the free radical polymerization anionic surfactants, cationic surfactant or non-ionic surfactant can be added, i.e., emulsion polymerization.

The anionic polymerizations usually carried out in an inert solvent such as for example toluene, hexane, cyclohexane, tetrahydrofuran (THF), 1,4-dioxane, 1,2-dimethoxyethane, pyridine, dimethyl sulfoxide under a water- and oxygen-free atmosphere. Alkaline metals such as for example Li, Na and K; and organometallic compounds such as for example buthyllithium, benzyllithium, trimethylsilylmethyllithium, phenylmagnesium bromide are used as the initiator for anionic polymerization. Such reactions are well known to those skilled in the art, and generally carried out at temperature in the range of −100° C. to 80° C., preferably −80° C. to 50° C.

The cationic polymerizations usually are carried out in an inert solvent such as for example toluene, hexane, cyclohexane, dichloromethane, dioxane. Broensted acids such as for example HCl, sulfuric acid, methanesulfonic acid, trifluoromethanesulfonic acid, fluorosulfonic acid; and Lewis acids such as for example BF₃, AlCl₃, TiCl₄, SnCl₄, FeCl₃ with co-catalysts such as for example HCl, H₂O, trifluoroacetic acid, methanol are used as the initiator for cationic polymerization. Such reactions are well known to those skilled in the art, and generally carried out at temperature in the range of −100° C. to 80° C., preferably −80° C. to 50° C. The preparation of the polymers by radical, anionic and cationic polymerizations is described in standard chemistry textbooks, for instance, G. Allen and J. C. Bevington, Comprehensive Polymer Science, Vol 3, Pergamon Press, 1989.

The polymer comprising repeating units derived from the compound of the formulae I, II and/or III can be also synthesized by controlled free radical polymerization such as for example, nitro-oxide mediated radical polymerization (NOR) described in C. J. Hawker, A. W. Bosman, E. Harth, Chem. Rev. 101, 3661 (2001), atom transfer radical polymerization (ATRP) described in K. Matyjaszewski, J. Xia, Chem. Rev. 101, 2921 (2001), radical addition-fragmentation chain transfer mediated polymerization (RAFT) described in G. Moad, Y. K. Chong, A. Postma, E. Rizzardo, S. H. Thang, Polymer 46 8458 (2005) and so on.

Homo-polymers comprising one repeating unit derived from the compound of the formulae I, II or III; and co-polymers comprising at least one repeating unit derived from the compound of the formulae I, II and/or III and optionally repeating units derived from ethylenically unsaturated compounds selected from the group of formula V can be prepared by the polymerization methods described above.

The compounds of the formulae I, II and III, and the polymers comprising repeating units derived from a compound of the formula I, II and/or III can be used as photosensitive acid donors.

Subject of the invention therefore is a composition comprising

(b) at least one polymer comprising at least one repeating unit derived from the compound of the formula I, II and/or III according to claim 1 and repeating units derived from ethylenically unsaturated compounds selected from the group of formula V as described above; as well as a composition comprising
(a) a compound which cures upon the action of an acid or a compound whose solubility is increased upon the action of an acid; and
(b) at least one compound of the formula I, II and/or III according to claim 1; and/or polymer comprising at least one repeating unit derived from the compound of the formula I, II and/or III according to claim 1 and optionally repeating units derived from ethylenically unsaturated compounds selected from the group of formula V as described above.

The compounds of the formulae I, II and III, and the polymers comprising repeating units derived from a compound of the formula I, II and/or III can be used as photosensitive acid donors in a photoresist. Resist systems can be prepared by image-wise irradiation of systems comprising compounds of formulae I, II and III and/or a polymer comprising repeating units derived from a compound of the formula I, II and/or III followed by a developing step.

The invention accordingly relates to a chemically amplified photoresist composition comprising
(a) a compound which cures upon the action of an acid; or a compound whose solubility is increased upon the action of an acid; and/or
(b) at least one compound of the formula I, II and/or III according to claim 1; and/or a polymer as described above.

In general,

(i) the composition according to the present invention comprises as component (a) a compound which cures upon the action of an acid; or a compound whose solubility is increased upon the action of an acid and, as component (b), a photolatent acid generator compound of the formula I, II and/or III; or
(ii) the composition comprises component (a) as described above and a polymer prepared by polymerizing or copolymerizing a compound of the formula I, II and/or III, not containing an acid-labile group; or
(iii) the composition comprises component (a) as described above and a polymer prepared by polymerizing or copolymerizing a compound of the formula I, II and/or III, containing an acid-labile group; or
(iv) the composition comprises a polymer prepared by polymerizing or copolymerizing a compound of the formula I, II and/or III, containing an acid-labile group.

In the latter case (iv), the polymer constitutes both, component (a) and component (b) as well.

A chemically amplified photoresist is understood to be a resist composition wherein the radiation sensitive component provides a catalytic amount of acid which subsequently catalyses a chemical reaction of at least one acid-sensitive component of the resist. Resulting is the induction of a solubility difference between the irradiated and non-irradiated areas of the resist. Because of the catalytic nature of this process one acid molecule can trigger reactions at multiple sites as it diffuses through the reactive polymer matrix, from one reaction site to the next, as long as it is not trapped or destroyed by any secondary reaction. Therefore, a small acid concentration is sufficient to induce a high difference in the solubility between exposed and unexposed areas in the resist. Thus, only a small concentration of the latent acid compound is necessary. As a result, resists with high contrast and high transparency at the exposure wavelength in optical imaging can be formulated, which in turn produce steep, vertical image profiles at high photosensitivity. However, as a result of this catalytic process, it is required that the latent acid catalysts are chemically and thermally very stable (as long as not irradiated) in order not to generate acid during resist storage or during processing, which—in most cases—requires a post exposure bake step to start or to complete the catalytic reaction which leads to the solubility differential. It is also required to have good solubility of the latent catalysts in the liquid resist formulation and the solid resist film to avoid any particle generation which would interfere with the application of these resists in microelectronic manufacturing processes.

In contrast, positive resist materials which are not based on the chemical amplification mechanism must contain a high concentration of the latent acid, because it is only the acid concentration which is generated from the latent acid under exposure which contributes to the increased solubility of the exposed areas in alkaline developer. Because small acid concentration has only a little effect on the change of the dissolution rate of such resist and the reaction proceeds typically without a post exposure bake here, the requirements regarding chemical and thermal stability of the latent acid are less demanding than for chemically amplified positive resists. These resists require also a much higher exposure dose to generate enough acid for achieving sufficient solubility in the alkaline developer in the exposed areas and also suffer from the relatively low optical transparency (due to the high concentration of latent acid necessary) and thus also lower resolution and sloped images. Resist compositions based on non-chemically amplified technology are therefore inferior in photosensitivity, resolution and image quality compared to chemically amplified resists.

From the above it becomes clear that chemical and thermal stability of a latent catalyst is vital for a chemically amplified resist and that latent acids which can work in a non-chemically amplified resist are not necessarily applicable to chemically amplified resists because of the different acid diffusion requirements, acid strength requirements and thermal and chemical stability requirements.

The difference in resist solubility between irradiated and non-irradiated sections that occurs as a result of the acid-catalysed reaction of the resist material during or after irradiation of the resist may be of two types depending upon which further constituents are present in the resist. If the compositions according to the invention comprise components that increase the solubility of the composition in the developer after irradiation, the resist is positive.

The invention accordingly relates to a chemically amplified positive photoresist.

If, on the other hand, the components of the formulation reduce the solubility of the composition after irradiation, the resist is negative.

The invention accordingly relates also to a chemically amplified negative photoresist.

Interesting is a chemically amplified positive photoresist composition as describe above, as component (b) comprising a polymer, comprising repeating units derived from a compound of the formula I, II and/or III as described above.

In particular preferred is a chemically amplified positive photoresist composition, wherein component (b) is at least one polymer comprising at least one repeating unit derived from a compound of the formula I, II and/or III according to claim 1; and

at least one repeating unit derived from ethylenically unsaturated compounds selected from the group of formula V as described above; and
at least one repeating unit derived from ethylenically unsaturated compounds selected from the group of formula VI,

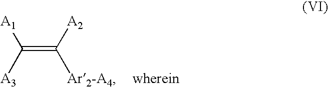

A₁, A₂, A₃, A₄ and Ar′₂ are as defined above.

A monomeric or polymeric compound which—in the unexposed areas—reduces the dissolution rate of an additionally present alkaline soluble binder resin in the resist formulation and which is essentially alkali-insoluble in the unexposed areas so that the resist film remains in the unexposed area after development in alkaline solution, but which is cleaved in the presence of acid, or is capable of being rearranged, in such a manner that its reaction product becomes soluble in the alkaline developer is referred to hereinafter as dissolution inhibitor.

The invention includes, as a special embodiment a chemically amplified positive alkaline-developable photoresist composition, comprising

(a1) at least one polymer having acid-labile groups which decompose in the presence of an acid and increase the solubility of the resist film in an aqueous alkaline developer solution in the exposed area and/or
(b) at least one compound of formula I, II or III, and/or a polymer comprising repeating units derived from a compound of the formula I, II and/or Ill.

A further embodiment of the invention is a chemically amplified positive alkaline-developable photoresist composition, comprising

(a2) at least one monomeric or oligomeric dissolution inhibitor having at least one acid-labile group which decomposes in the presence of acid and increases the solubility in an aqueous alkaline developer solution and at least one alkali-soluble polymer and/or,
(b) at least one compound of formula I, II or III and/or a polymer comprising repeating units derived from a compound of the formula I, II and/or Ill.

Another specific embodiment of the invention resides in a chemically amplified positive alkaline-developable photoresist composition, comprising

(a1) at least one polymer having acid labile groups which decompose in the presence of an acid and increase the solubility in an alkaline developer in the exposed area;
(a2) at least one a monomeric or oligomeric dissolution inhibitor, having at least one acid labile group, which decomposes in the presence of an acid and increase the alkaline solubility in the exposed area;
(a3) at least one an alkali-soluble monomeric, oligomeric or polymeric compound at a concentration which still keeps the resist film in the unexposed area essentially insoluble in the alkaline developer, and/or
(b) at least one compound of formula I, II or III and/or a polymer comprising repeating units derived from a compound of the formula I, II and/or III.

The invention therefore pertains to a chemically amplified photoresist composition, comprising

(a1) at least one polymer having an acid-labile group which decomposes in the presence of an acid to increase the solubility in aqueous alkaline developer solution and/or
(a2) at least one monomeric or oligomeric dissolution inhibitor having an acid-labile group which decomposes in the presence of an acid to increase the solubility in aqueous alkaline developer solution and/or
(a3) at least one alkali-soluble monomeric, oligomeric or polymeric compound; and
(b) as photosensitive acid donor, at least one compound of formula I, II or III and/or a polymer comprising repeating units derived from a compound of the formula I, II and/or III.

Preferably the composition is a chemically amplified positive photoresist composition, comprising
(a1) at least one polymer having an acid-labile group which decomposes in the presence of an acid to increase the solubility in aqueous alkaline developer solution; and/or
(a2) at least one monomeric or oligomeric dissolution inhibitor having an acid-labile group which decomposes in the presence of an acid to increase the solubility in aqueous alkaline developer solution; and/or
(a3) at least one alkali-soluble monomeric, oligomeric or polymeric compound; and
(b) at least one compound of the formula I, II and/or III; and/or a polymer comprising at least one repeating unit derived from the compound of the formula I, II and/or III and at least one repeating unit derived from ethylenically unsaturated compounds selected from the group of formula VI as described above, and optionally repeating units derived from ethylenically unsaturated compounds selected from the group of formula V as described above.

The compositions may comprise additionally to the component (b) other photosensitive acid donors and/or (c) other additives.

A polymer having acid labile groups which decompose in the presence of an acid and increase the solubility in an alkaline developer can comprise photosensitive acid donor groups in the polymer. Such polymer can simultaneously work as photosensitive acid donor and as polymer whose solubility is increased upon the action of an acid in a chemically amplified positive photoresist composition.

The present invention pertains to a chemically amplified positive photoresist composition, comprising

(b) as photosensitive acid donor and as compound whose solubility is increased upon the action of an acid, at least one compound of the formula I, II and/or III; and/or a polymer comprising at least one repeating unit derived from a compound of the formula I, II and/or III and repeating units derived from ethylenically unsaturated compounds selected from the group of formula V as described above.

Chemically amplified positive resist systems are described, for example, in E. Reichmanis, F. M. Houlihan, O, Nalamasu, T. X. Neenan, Chem. Mater. 1991, 3, 394; or in C. G. Willson, “Introduction to Microlithography, 2nd. Ed.; L. S. Thompson, C. G. Willson, M. J. Bowden, Eds., Amer. Chem. Soc., Washington D.C., 1994, p. 139.

Suitable examples of acid-labile groups which decompose in the presence of an acid to produce aromatic hydroxy groups, carboxylic groups, keto groups and aldehyde groups and increase the solubility in aqueous alkaline developer solution are, for example, alkoxyalkyl ether groups, tetrahydrofuranyl ether groups, tetrahydropyranyl ether groups, tert-alkyl ester groups, trityl ether groups, silyl ether groups, alkyl carbonate groups as for example tert.-butyloxycarbonyloxy-, trityl ester groups, silyl ester groups, alkoxymethyl ester groups, cumyl ester groups, acetal groups, ketal groups, tetrahydropyranyl ester groups, tetrafuranyl ester groups, tertiary alkyl ether groups, tertiary alkyl ester groups, and the like. Examples of such group include alkyl esters such as methyl ester and tert-butyl ester, acetal type esters such as methoxymethyl ester, ethoxymethyl enter, 1-ethoxyethyl ester, 1-isobutoxyethyl ester, 1-isopropoxyethyl ester, 1-ethoxypropyl ester, 1-(2-methoxyethoxy)ethyl ester, 1-(2-acetoxyethoxy)ethyl ester, 1-[2-(1-adamantyloxy)ethoxy]ethyl ester, 1-[2-(1-adamantylcarbonyloxy)ethoxy]ethyl ester, tetrahydro-2-furyl ester and tetrahydro-2-pyranyl ester, and alicyclic ester such as isobornyl ester.

The polymer having functional groups capable of decomposing by the action of an acid to enhance solubility of the resist film comprising this polymer in an alkaline developing solution, which can be incorporated in the positive resist according to the present invention, may have the acid-labile groups in the backbone and/or side chains thereof, preferably in side chains thereof.

The polymer having acid-labile groups suitable for the use in the present invention can be obtained with a polymer analogous reaction where the alkaline soluble groups are partially or completely converted into the respective acid labile groups or directly by (co)-polymerization of monomers which have the acid labile groups already attached, as is for instance disclosed in EP 254853, EP 878738, EP 877293, JP-A-2-25850, JP-A-3-223860, and JP-A-4-251259.

The polymers which have acid labile groups pendant to the polymer backbone, in the present invention preferably are polymers which have, for example silylether, acetal, ketal and alkoxyalkylester groups (called “low-activation energy blocking groups”) which cleave completely at relatively low post exposure bake temperatures (typically between room temperature and 110° C.) and polymers which have, for example, tert-butylester groups or tert.-butyloxycarbonyl (TBOC) groups or other ester groups which contain a secondary or tertiary carbon atom next to the oxygen atom of the ester bond (called “high-activation energy blocking groups”) which need higher bake temperatures (typically>110° C.) in order to complete the deblocking reaction in the presence of acid. Hybrid systems can also be applied, wherein, both, high activation energy blocking groups as well as low activation energy blocking groups are present within one polymer. Alternatively, polymer blends of polymers, each utilizing a different blocking group chemistry, can be used in the photosensitive positive resist compositions according to the invention.

Preferred polymers which have acid labile groups are polymers and co-polymers comprising the following distinct monomer types:

1) monomers that contain acid-labile groups which decompose in the presence of an acid to increase the solubility in aqueous alkaline developer solution and
2) monomers that are free of acid labile groups and free of groups that contribute to the alkaline solubility and/or
3) monomers that contribute to aqueous alkaline solubility of the polymer.

Examples of monomers of type 1) are:

non-cyclic or cyclic secondary and tertiary-alkyl (meth)acrylates such as butyl acrylate, including t-butyl acrylate, butyl methacrylate, including t-butyl methacrylate, 3-oxocyclohexyl (meth)acrylate, tetrahydropyranyl (meth)acrylate, 2-methyl-adamantyl (meth)acrylate, cyclohexyl (meth)acrylate, norbornyl (meth)acrylate, (2-tetrahydropyranyl)oxynorbonylalcohol acrylates, (2-tetrahydropyranyl)oxymethyltricyclododecanemethanol methacrylates, trimethylsilylmethyl (meth)acrylate, (2-tetrahydropyranyl)oxynorbonylalcohol acrylates, (2-tetrahydropyranyl)oxymethyltricyclododecanemethanol methacrylates, trimethylsilylmethyl (meth)acrylate o-/m-/p-(3-oxocyclohexyloxy)styrene, o-/m-/p-(1-methyl-1-phenylethoxy)styrene, o-/m-/p-tetrahydropyranyloxystyrene, o-/m-/p-adamantyloxystyrene, o-/m-/p-cyclohexyloxystyrene, o-/m-/pnorbornyloxystyrene, non-cyclic or cyclic alkoxycarbonylstyrenes such as o-/m-/p-butoxycarbonylstyrene, including p-t-butoxycarbonylstyrene, o-/m-/p-(3-oxocyclohexyloxycarbonyl)styrene, o-/m-/p-(1-methyl-1-phenylethoxycarbonyl)styrene, o-/m-/p-tetrahydropyranyloxycarbonylstyrene, o-/m-/p-adamantyloxycarbonylstyrene, o-/m-/p-cyclohexyloxycarbonylstyrene, o-/m-/p-norbornyloxycarbonylstyrene, non-cyclic or cyclic alkoxycarbonyloxystyrenes such as o-/m-/p-butoxycarbonyloxystyrene, including p-t-butoxycarbonyloxystyrene, o-/m-/p-(3-oxocyclohexyloxycarbonyloxy)styrene, o-/m-/p-(1-methyl-1-phenylethoxycarbonyloxy)styrene, o-/m-/p-tetrahydropyranyloxycarbonyloxystyrene, o-/m-/p-adamantyloxycarbonyloxystyrene, o-/m-/p-cyclohexyloxycarbonyloxystyrene, o-/m-/p-norbornyloxycarbonyloxystyrene, non-cyclic or cyclic alkoxycarbonylalkoxystyrenes such as o/m/p-butoxycarbonylmethoxystyrene, p-t-butoxycarbonylmethoxystyrene, o-/m-/p-(3-oxocyclohexyloxycarbonylmethoxy)styrene, o-/m-/p-(1-methyl-1-phenylethoxycarbonylmethoxy)styrene, o-/m-/p-tetrahydropyranyloxycarbonylmethoxystyrene, o-/m-/p-adamantyloxycarbonylmethoxystyrene, o-/m-/p-cyclohexyloxycarbonylmethoxystyrene, o-/m-/p-norbornyloxycarbonylmethoxystyrene, trimethylsiloxystyrene, dimethyl(butyl)siloxystyrene, unsaturated alkyl acetates such as isopropenyl acetate and the derivatives of thereof.

Monomers of type 1) bearing low activation energy acid labile groups include, for example, p- or m-(1-methoxy-1-methylethoxy)-styrene, p- or m-(1-methoxy-1-methylethoxy)-methylstyrene, p- or m-(1-methoxy-1-methylpropoxy)styrene, p- or m-(1-methoxy-1-methylpropoxy) methylstyrene, p- or m-(1-methoxyethoxy)-styrene, p- or m-(1-methoxyethoxy)-methylstyrene, p- or m-(1-ethoxy-1-methylethoxy)styrene, p- or m-(1-ethoxy-1-methylethoxy)-methylstyrene, p- or m-(1-ethoxy-1-methylpropoxy)styrene, p- or m-(1-ethoxy-1-methylpropoxy)-methylstyrene, p- or m-(1-ethoxyethoxy)styrene, p- or m-(1-ethoxyethoxy)-methylstyrene, p-(1-ethoxyphenyl-ethoxy)styrene, p- or m-(1-n-propoxy-1-methylethoxy)styrene, p- or m-(1-n-propoxy-1-methylethoxy)-methylstyrene, p- or m-(1-n-propoxyethoxy)styrene, p- or m-(1-n-propoxyethoxy)-methylstyrene, p- or m-(1-isopropoxy-1-methylethoxy)styrene, p- or m-(1-isopropoxy-1-methylethoxy)-methylstyrene, p- or m-(1-isopropoxyethoxy)styrene, p- or m-(1-isopropoxyethoxy)-methylstyrene, p- or m-(1-isopropoxy-1-methylpropoxy)styrene, p- or m-(1-isopropoxy-1-methylporpoxy)-methylstyrene, p- or m-(1-isopropoxypropoxy)styrene, p- or m-(1-isopropoxyporpoxy)-methylstyrene, p- or m-(1-n-butoxy-1-methylethoxy)styrene, p- or m-(1-n-butoxyethoxy)styrene, p- or m-(1-isobutoxy-1-methylethoxy)styrene, p- or m-(1-tert-butoxy-1-methylethoxy)styrene, p- or m-(1-n-pentoxy-1-methylethoxy)styrene, p- or m-(1-isoamyloxy-1-methylethoxy)styrene, p- or m-(1-n-hexyloxy-1-methylethoxy)styrene, m-(1-cyclohexyloxy-1-methylethoxy)styrene, p- or m-(1-trimethylsilyloxy-1-methylethoxy)styrene, p- or m-(1-trimethylsilyloxy-1-methylethoxy)-methylstyrene, p- or m-(1-benzyloxy-1-methylethoxy)styrene, p- or m-(1-benzyloxy-1-methylethoxy)-methylstyrene, p- or m-(1-methoxy-1-methylethoxy)styrene, p- or m-(1-methoxy-1-methylethoxy)-methylstyrene, p- or m-(1-trimethylsilyloxy-1-methylethoxy)styrene p- or m-(1-trimethylsilyloxy-1-methylethoxy)-methylstyrene. Other examples of polymers having alkoxyalkylester acid labile groups are given in U.S. Pat. No. 5,225,316 and EP 829766. Examples of polymers with acetal blocking groups are given in U.S. Pat. No. 5,670,299, EP 780732, U.S. Pat. No. 5,627,006, U.S. Pat. No. 5,558,976, U.S. Pat. No. 5,558,971, U.S. Pat. No. 5,468,589, EP 704762, EP 762206, EP 342498, EP 553737 and described in ACS Symp. Ser. 614, Microelectronics Technology, pp. 35-55 (1995) and J. Photopolymer Sci. Technol. Vol. 10, No. 4 (1997), pp. 571-578. The polymer used in the present invention is not limited thereto.

With respect to polymers having acetal groups as acid-labile groups, it is possible to incorporate acid labile crosslinks as for example described in H.-T. Schacht, P. Falcigno, N. Muenzel, R. Schulz, and A. Medina, ACS Symp. Ser. 706 (Micro- and Nanopatterning Polymers), p. 78-94, 1997; H.-T. Schacht, N. Muenzel, P. Falcigno, H. Holzwarth, and J. Schneider, J. Photopolymer Science and Technology, Vol. 9, (1996), 573-586. This crosslinked system is preferred from the standpoint of heat resistance of the resist patterns.

Monomers with high activation energy acid labile groups are, for example, p-tert.-butoxycarbonyloxystyrene, tert.-butyl-acrylate, tert.-butyl-methacrylate, 2-methyl-2-adamantyl-methacrylate, isobornyl-methacrylate.

Monomers of type 1) suitable for ArF resist technology in particular include, for example, 2-methyl-2-adamantyl acrylate, 2-ethyl-2-adamantyl acrylate, 2-n-butyl-2-adamantyl acrylate, 2-n-butyl-2-adamantyl methacrylate, 2-methyl-2-adamantyl methacrylate, 2-ethyl-2-adamantyl methacrylate2-(1-adamantyl)isopropyl methacrylate, 2-(1-adamantyl)isopropyl acrylate, 2-(1-adamantyl)isobutyl methacrylate, 2-(1-adamantyl) isobutyl acrylate, t-butyl methacrylate, t-butyl acrylate, 1-methylcyclohexyl methacrylate, 1-methylcyclohexyl acrylate, 1-ethylcyclohexyl methacrylate, 1-ethylcyclohexyl acrylate, 1-(n-propyl)cyclohexyl methacrylate, 1-(n-propyl)cyclohexyl acrylate, tetrahydro-2-methacryloyloxy-2H-pyran and tetrahydro-2-acryloyloxy-2H-pyran. Other monomers comprising acid-labile adamantyl moieties are disclosed in JP-A-2002-1265530, JP-A-2002-338627, JP-A-2002-169290, JP-A-2002-241442, JP-A-2002-145954, JP-A-2002-275215, JP-A-2002-156750, JP-A-2002-268222, JP-A-2002-169292, JP-A-2002-162745, JP-A-2002-301161, WO02/06901A2, JP-A-2002-311590, JP-A-2002-182393, JP-A-2002-371114, JP-A-2002-162745.

Particular olefins with acid labile-group are also suitable for ArF resist technology as shown in, for example, JP-A-2002-308938, JP-A-2002-308869, JP-A-2002-206009, JP-A-2002-179624, JP-A-2002-161116.

Examples of comonomers according to type 2) are:

aromatic vinyl monomers, such as styrene, α-methylstyrene, acetoxystyrene, α-methylnaphthylene, acenaphthylene, vinyl alicyclic compounds such as vinyl norbornane, vinyl adamantane. vinyl cyclohexane, alkyl (meth)acrylates such as methyl methacrylate, (meth)acrylonitrile, vinylcyclohexane, vinylcyclohexanol, itaconic anhydride, as well as maleic anhydride.

Comonomers according to type 2) suitable for ArF resist technology in particular include, for example, alpha-acryloyloxy-gamma-butyrolactone, alpha-methacryloyloxy-gamma-butyrolactone, alpha-acryloyloxy-beta, beta-dimethyl-gamma-butyro-lactone, alpha-methacryloyloxy-beta, beta-dimethyl-gamma-butyrolactone, alpha-acryloyloxy-alpha-methyl-gamma-butyrolactone, alpha-methacryloyloxy-alpha-methyl-gamma-butyrolactone, beta-acryloyloxy-gamma, beta-methacryloyloxy-alpha-methyl-gamma-butyrolactone, 5-acryloyloxy-2,6-norbornanecarbolactone, 5-methacryloyloxy-2,6-norbolnanecarbolactone, 2-norbornene, methyl 5-norbornene-2-carboxylate, tert-butyl 5-norbornene-2-carboxylate, 1-cyclohexyl-1-methylethyl 5-norbornene-2-carboxylate, 1-(4-methylcyclohexyl)-1-methylethyl 5-norbornene-2-carboxylate, 1-methyl-1-(4-oxocyclohexyl)ethyl 5-norbornene-2-carboxylate, 1-(1-adamatyl)-1-methylethyl 5-norbornene-2-carboxylate, 1-methylcyclohexyl 5-norbornene-2-carboxylate, 2-methyl-2-adamantyl 5-norbornene-2-carboxylate, 2-ethyl-2-adamantyl 5-norbornene-2-carboxylate, 5-norbornene-2,3-dicarboxylic acid anhydrate, 2(5H)-furanone, 3-vinyl-gamma-butyrolactone, 3-methacryloyloxybicyclo[4,3,0]nonane, 3-acryloyloxybicyclo[4,3,0]nonane, 1-adamantyl methacrylate, 1-adamantyl acrylate, 3-methacryloyloxymethyltetracyclo[4,4,0,1^{2.5},1^{7.10}]dodecane, 3-acryloyloxymethyltetracyclo[4,4,0,1^{2.5},1^{7.10}]dodecane, 2-methacryloyloxynorbornane, 2-acryloyloxynorbornane, 2-methacryloyloxyisobornane, 2-acryloyloxyisobornane, 2-methacryloyloxymethylnorbornane, 2-acryloyloxymethylnorbornane.

Examples of comonomers according to type 3) are:

vinyl aromatic compounds such as hydroxystyrene, acrylic acid compounds such as methacrylic acid, ethylcarbonyloxystyrene and derivatives of thereof. These polymers are described, for example, in U.S. Pat. No. 5,827,634, U.S. Pat. No. 5,625,020, U.S. Pat. No. 5,492,793, U.S. Pat. No. 5,372,912, EP 660187, U.S. Pat. No. 5,679,495, EP 813113 and EP 831369. Further examples are crotonic acid, isocrotonic acid, 3-butenoic acid, acrylic acid, 4-pentenoic acid, propiolic acid, 2-butynoic acid, maleic acid, fumaric acid, and acetylenecarboxylic acid. The polymer used in the present invention is not limited thereto.

Comonomers according to type 3) suitable for ArF resist technology in particular include, for example, 3-hydroxy-1-adamantyl acrylate, 3-hydroxy-1-adamantyl methacrylate, 3,5-dihydroxy-1-adamantyl acrylate, 3,5-dihydroxy-1-adamantyl methacrylate, 2-hydroxy-5-norbornene, 5-norbornene-2-carboxylic acid, 1-(4-hydroxycyclohexyl)-1-methylethyl 5-norbornene-2-carboxylate, 2-hydroxy-1-ethyl 5-norbornene-2-carboxylate, 5-norbornene-2-methanol, 8-Hydroxymethyl-4-methacryloyloxymethyltricyclo[5.2.1.0^{2.6}]decane, 8-Hydroxymethyl-4-acryloyloxymethyltricyclo[5.2.1.0^{2.6}]decane, 4-Hydroxymethyl-8-methacryloyloxymethyltricyclo[5.2.1.0^{2.6}]decane, 4-Hydroxymethyl-8-acryloyloxymethyltricyclo[5.2.1.0^{2.6}]decane.

Other monomers comprising lactone moieties suitable for ArF technology are disclosed in, for example, JP-A-2002-6502, JP-A-2002-145955, EP1127870A1, JP-A-2002-357905, JP-A-2002-296783. Other olefins suitable for ArF technology are published in, for example, JP-A-2002-351078, JP-A-2002-234918, JP-A-2002-251009, EP1127870A1, JP-A-2002-328475, JP-A-2002-278069, JP-A-2003-43689, JP-A-2002-202604, WO01/86353, JP-A-2002-23371, JP-A-2002-72484, JP-A-2002-202604, JP-A-2001-330959, JP-A-2002-3537, JP-A-2002-30114, JP-A-2002-278071, JP-A-2002-251011, JP-A-2003-122010, JP-A-2002-139837, JP-A-2003-195504, JP-A-2001-264984, JP-A-2002-278069, JP-A-2002-328475, U.S. Pat. No. 6,379,861, U.S. Pat. No. 6,599,677, US2002/119391, U.S. Pat. No. 6,277,538, US2003/78354.

The content of acid labile monomers in the polymer may vary over a wide range and depends on the amount of the other comonomers and the alkaline solubility of the deprotected polymer. Typically, the content of monomers with acid labile groups in the polymer is between 5 and 60 mol %. If the content is too small, too low development rates and residues of the resist in the exposed areas result. If the content of acid labile monomers is too high, resist patterns are poorly defined (eroded) after development and narrow features cannot be resolved anymore and/or the resist looses its adhesion to the substrate during development. Preferably the copolymers which have acid labile groups have a M_{w} of from about 3′000 to about 200′000, more preferably from about 5′000 to about 50′000 with a molecular weight distribution of about 3 or less, more preferably a molecular weight distribution of about 2 or less. Non-phenolic polymers, e.g. a copolymer of an alkyl acrylate such as t-butyl acrylate or t-butyl-methacrylate and a vinyl alicyclic compound, such as a vinyl norbonanyl or vinyl cyclohexanol compound, also may be prepared by such free radical polymerization or other known procedures and suitably will have a M_{W} of from about 8′000 to about 50′000, and a molecular weight distribution of about 3 or less.

Other comonomers may suitably be added in an appropriate amount for the purpose of controlling the glass transition point of the polymer and the like.

In the present invention a mixture of two or more polymers having acid-labile groups may be used. For example, use may be made of a mixture of a polymer having acid-labile groups, which are cleaved very easily, such as acetal groups or tetrahydropyranyloxy-groups and a polymer having acid-cleavable groups, that are less easily cleaved, such as for example tertiary alkyl ester groups. Also, acid cleavable groups of different size can be combined by blending two or more polymers having different acid cleavable groups, such as a tert-butylester group and 2-methyl-adamantyl group or an 1-ethoxy-ethoxy group and a tetrahydropyranyloxy group. A mixture of a non-crosslinked resin and a crosslinked resin may also be used. The amount of these polymers in the present invention is preferably from 30 to 99% by weight, more preferably from 50 to 98% by weight, based on the total amount of all solid components. An alkali-soluble resin or monomeric or oligomeric compound having no acid-labile groups may be further incorporated into the composition in order to control the alkali solubility.

Examples of polymer blends with polymers having different acid-labile groups are given in EP 780732, EP 679951 and U.S. Pat. No. 5,817,444.

Preferably monomeric and oligomeric dissolution inhibitors (a2) are used in the present invention.

The monomeric or oligomeric dissolution inhibitor having the acid-labile group for use in the present invention is a compound which has at least one acid-labile group in the molecular structure, which decomposes in the presence of acid to increase the solubility in aqueous alkaline developer solution. Examples are alkoxymethyl ether groups, tetrahydrofuranyl ether groups, tetrahydropyranyl ether groups, alkoxyethyl ether groups, trityl ether groups, silyl ether groups, alkyl carbonate groups, trityl ester groups, silyl ester groups, alkoxymethyl ester groups, vinyl carbamate groups, tertiary alkyl carbamate groups, trityl amino groups, cumyl ester groups, acetal groups, ketal groups, tetrahydropyranyl ester groups, tetrafuranyl ester groups, tertiary alkyl ether groups, tertiary alkyl ester groups, and the like. The molecular weight of the acid-decomposable dissolution inhibitive compound for use in the present invention is 3′000 or lower, preferably from 100 to 3′000, more preferably from 200 to 2′500.

Examples of monomeric and oligomeric dissolution inhibitors having acid-labile groups are described as formulae (I) to (XVI) in EP 0831369. Other suitable dissolution inhibitors having acid-labile groups are shown in U.S. Pat. No. 5,356,752, U.S. Pat. No. 5,037,721, U.S. Pat. No. 5,015,554, JP-A-1-289946, JP-A-1-289947, JP-A-2-2560, JP-A-3-128959, JP-A-3-158855, JP-A-3-179353, JP-A-3-191351, JP-A-3-200251, JP-A-3-200252, JP-A-3-200253, JP-A-3-200254, JP-A-3-200255, JP-A-3-259149, JA-3-279958, JP-A-3-279959, JP-A-4-1650, JP-A-4-1651, JP-A-11260, JP-A-4-12356, JP-A-4-123567, JP-A-1-289946, JP-A-3-128959, JP-A-3-158855, JP-A-3-179353, JP-A-3-191351, JP-A-3-200251, JP-A-3-200252, JP-A-3-200253, JP-A-3-200254, JP-A-3-200255, JP-A-3-259149, JP-A-3-279958, JP-A-3-279959, JP-A-4-1650, JP-A-4-1651, JP-A-11260, JP-A-4-12356, JP-A-4-12357 and Japanese Patent Applications Nos. 3-33229, 3-230790, 3-320438, 4-254157, 4-52732, 4-103215, 4-104542, 4-107885, 4-107889, 4-152195, 4-254157, 4-103215, 4-104542, 4-107885, 4-107889, and 4-152195.

The composition can also contain polymeric dissolution inhibitors, for example, polyacetals as described for example in U.S. Pat. No. 5,354,643 or poly-N,O-acetals for example those described in U.S. Pat. No. 5,498,506, either in combination with an alkaline soluble polymer, or in combination with a polymer containing acid labile groups which increase the solubility of the resist film in the developer after exposure, or with a combination of both types of polymers.

In the case where the dissolution inhibitor having acid-labile groups is used in the present invention in combination with the sulfonate derivatives of formula I, II or III, the alkali-soluble polymer and/or the polymer having acid-labile groups, the amount of the dissolution inhibitor is from 3 to 55% by weight, preferably from 5 to 45% by weight, most preferably from 10 to 35% by weight, based on the total amount of all solid components of the photosensitive composition.

A polymer soluble in an aqueous alkali solution (a3) is preferably used in the present invention. Examples of these polymers include novolak resins, hydrogenated novolak resins, acetone-pyrogallol resins, poly(o-hydroxystyrene), poly(m-hydroxystyrene), poly(p-hydroxystyrene), hydrogenated poly(hydroxystyrene)s, halogen- or alkyl-substituted poly(hydroxystyrene)s, hydroxystyrene/N-substituted maleimide copolymers, o/p- and m/p-hydroxystyrene copolymers, partially o-alkylated poly(hydroxystyrene)s, [e.g., o-methylated, o-(1-methoxy)ethylated, o-(1-ethoxy)ethylated, o-2-tetrahydropyranylated, and o-(t-butoxycarbonyl)methylated poly(hydroxystyrene)s having a degree of substitution of from 5 to 30 mol % of the hydroxyl groups], o-acylated poly(hydroxystyrene)s [e.g., o-acetylated and o-(t-butoxy)carbonylated poly(hydroxystyrene)s having a degree of substitution of from 5 to 30 mol % of the hydroxyl groups], styrene/maleic anhydride copolymers, styrene/hydroxystyrene copolymers, α-methylstyrene/hydroxystyrene copolymers, carboxylated methacrylic resins, and derivatives thereof. Further suitable are poly (meth)acrylic acid [e.g. poly(acrylic acid)], (meth)acrylic acid/(meth)acrylate copolymers [e.g. acrylic acid/methyl acrylate copolymers, methacrylic acid/methyl methacrylate copolymers or methacrylic acid/methyl methacrylate/t-butyl methacrylate copolymers], (meth)acrylic acid/alkene copolymers [e.g. acrylic acid/ethylene copolymers], (meth)acrylic acid/(meth)acrylamide copolymers [e.g. acrylic acid/acrylamide copolymers], (meth)acrylic acid/vinyl chloride copolymers [e.g. acrylic acid/vinyl chloride copolymers], (meth)acrylic acid/vinyl acetate copolymer [e.g. acrylic acid/vinyl acetate copolymers], maleic acid/vinyl ether copolymers [e.g. maleic acid/methyl vinyl ether copolymers], maleic acid mono ester/methyl vinyl ester copolymers [e.g. maleic acid mono methyl ester/methyl vinyl ether copolymers], maleic acid/(meth)acrylic acid copolymers [e.g. maleic acid/acrylic acid copolymers or maleic acid/methacrylic acid copolymers], maleic acid/(meth)acrylate copolymers [e.g. maleic acid/methyl acrylate copolymers], maleic acid/vinyl chloride copolymers, maleic acid/vinyl acetate copolymers and maleic acid/alkene copolymers [e.g. maleic acid/ethylene copolymers and maleic acid/1-chloropropene copolymers]. However, the alkali-soluble polymer for use in the present invention should not be construed as being limited to these examples.

Especially preferred alkali-soluble polymers (a3) are novolak resins, poly(o-hydroxystyrene), poly(m-hydroxystyrene), poly(p-hydroxystyrene), copolymers of the respective hydroxystyrene monomers, for example with p-vinylcyclohexanol, alkyl-substituted poly(hydroxystyrene)s, partially o- or m-alkylated and o- or m-acylated poly(hydroxystyrene)s, styrene/hydroxystyrene copolymer, and α-methylstyrene/hydroxystyrene copolymers. The novolak resins are obtained by addition-condensing one or more given monomers as the main ingredient with one or more aldehydes in the presence of an acid catalyst.

Examples of monomers useful in preparing alkaline soluble resins include hydroxylated aromatic compounds such as phenol, cresols, i.e., m-cresol, p-cresol, and o-cresol, xylenols, e.g., 2,5-xylenol, 3,5-xylenol, 3,4-xylenol, and 2,3-xylenol, alkoxyphenols, e.g., p-methoxyphenol, m-methoxyphenol, 3,5-dimethoxyphenol, 2-methoxy-4-methylphenol, m-ethoxyphenol, p-ethoxyphenol, m-propoxyphenol, p-propoxyphenol, m-butoxyphenol, and p-butoxyphenol, dialkylphenols, e.g., 2-methyl-4-isopropylphenol, and other hydroxylated aromatics including m-chlorophenol, p-chlorophenol, o-chlorophenol, dihydroxybiphenyl, bisphenol A, phenylphenol, resorcinol, and naphthol. These compounds may be used alone or as a mixture of two or more thereof. The main monomers for novolak resins should not be construed as being limited to the above examples.

Examples of the aldehydes for polycondensation with phenolic compounds to obtain novolaks include formaldehyde, p-formaldehyde, acetaldehyde, propionaldehyde, benzaldehyde, phenylacetaldehyde, α-phenylpropionaldehyde, β-phenylpropionaldehyde, o-hydroxybenzaldehyde, m-hydroxybenzaldehyde, p-hydroxybenzaldehyde, o-chlorobenzaldehyde, m-chlorobenzaldehyde, p-chlorobenzaldehyde, o-nitrobenzaldehyde, m-nitrobenzaldehyde, o-methylbenzaldehyde, m-methylbenzaldehyde, p-methylbenzaldehyde, p-ethylbenzaldehyde, p-n-butylbenzaldehyde, furfural, chloroacetaldehyde, and acetals derived from these, such as chloroacetaldehyde diethyl acetal. Preferred of these is formaldehyde.

These aldehydes may be used alone or in combination of two or more thereof. Examples of the acid catalyst include hydrochloric acid, sulfuric acid, formic acid, acetic acid, and oxalic acid.

The weight-average molecular weight of the thus-obtained novolak resin suitably is from 1′000 to 30′000. If the weight-average molecular weight thereof is lower than 1′000, the film reduction at unexposed parts during development is liable to be large. If the weight-average molecular weight there of exceeds 50′000, the developing rate may be too low. The especially preferred range of the molecular weight of the novolak resin is from 2′000 to 20′000.

The poly(hydroxystyrene)s and derivatives and copolymers thereof shown above as alkali-soluble polymers other than novolak resins each have a weight-average molecular weight of 2′000 or higher, preferably from 4′000 to 200′000, more preferably from 5′000 to 50′000. From the standpoint of obtaining a polymer film having improved heat resistance, the weight-average molecular weight thereof is desirably at least 5′000 or higher.

Weight-average molecular weight in the context of the present invention is meant to be the one determined by gel permeation chromatography and calibrated for with polystyrene standard.

In the present invention the alkali-soluble polymers may be used as a mixture of two or more thereof. In the case where a mixture of an alkali-soluble polymer and the polymer having groups which decompose by the action of an acid to enhance solubility in an alkaline developing solution is used, the addition amount of the alkali-soluble polymer is preferably up to 80% by weight, more preferably up to 60% by weight, most preferably up to 40% by weight, based on the total amount of the photosensitive composition (excluding the solvent). The amount exceeding 80% by weight is undesirable because the resist pattern suffers a considerable decrease in thickness, resulting in poor images and low resolution.

In the case where an alkali-soluble polymer is used together with a dissolution inhibitor, without the polymer having groups which decompose by the action of an acid, to enhance solubility in an alkaline developing solution, the amount of the alkali-soluble polymer is preferably from 40% to 90% by weight, more preferably from 50 to 85% by weight, most preferably 60 to 80% by weight. If the amount thereof is smaller than 40% by weight, undesirable results such as reduced sensitivity are caused. On the other hand, if it exceeds 90% by weight, the resist pattern suffers a considerable decrease in film thickness, resulting in poor resolution and image reproduction.

The use of the sulfonate derivatives according to the invention in chemically amplified systems, which operates on the principle of the removal of a protecting group from a polymer, generally produces a positive resist. Positive resists are preferred over negative resists in many applications, especially because of their higher resolution. There is, however, also interest in producing a negative image using the positive resist mechanism, in order to combine the advantages of the high degree of resolution of the positive resist with the properties of the negative resist. This can be achieved by introducing a so-called image-reversal step as described, for example, in EP 361906. For this purpose, the image-wise irradiated resist material is before the developing step treated with, for example, a gaseous base, thereby image-wise neutralizing the acid which has been produced. Then, a second irradiation, over the whole area, and thermal aftertreatment are carried out and the negative image is then developed in the customary manner.

The compounds of the formula I, II and III, and the polymers comprising repeating units derived from a compound of the formula I, II and/or III according to the present invention are in particular suitable as photolatent acids in the ArF resist technology, i.e. a technology using ArF excimer lasers (193 nm) for the imaging step. This technology requests the use of specific polymers/copolymers. Suitable formulations and the preparation of suitable polymer/copolymers are for example published in

Proceeding of SPIE 2438, 474 (1995); Proceeding of SPIE 3049, 44 (1997); Proceeding of SPIE 3333, 144 (1998); J. Photopolym. Sci. Technol. 14, 631 (2001); Proceeding of SPIE 3333, 546 (1998); J. Photopolym. Sci. Technol. 13, 601 (2000); JP2001-242627A; JP2001-290274A; JP2001-235863A; JP2001-228612A; Proceeding of SPIE 3333, 144 (1998); JP2001-5184A, commercially available as Lithomax alpha-7K from Mitsubishi Rayon; JP2001-272783A; U.S. patent application Ser. No. 09/413,763 (filed Oct. 7, 1999); EP 1091249; JP2000-292917A; JP2003-241385A; J. Photopolym. Sci. Technol. 14, 631 (2001); Proceeding of SPIE 3333, 11 (1998); ACS1998 (University of Texas); JP2001-290274A; JP2001-235863A; JP2001-228612A; Proceeding of SPIE 3999, 13 (2000); JP2001-296663A; U.S. patent application Ser. No. 09/567,814 (filed 2000.5.9); EP 1128213; Proceeding of SPIE 3049, 104 (1997); J. Photopolym. Sci. Technol. 10, 521 (1997); JP2001-290274A; JP2001-235863A; JP2001-228612A; Proceeding of SPIE 4345, 680 (2001); J. Vac. Sci. Technol. B 16(6), p. 3716, 1998; Proceeding of SPIE 2724, 356 (1996); Proceeding of SPIE 4345, 67 (2001); Proceeding of SPIE 3333, 546 (1998); Proceeding of SPIE 4345, 87 (2001); Proceeding of SPIE 4345, 159 (2001); Proceeding of SPIE 3049, 92 (1997); Proceeding of SPIE 3049, 92 (1997); Proceeding of SPIE 3049, 92 (1997); Proceeding of SPIE 3999, 2 (2000); Proceeding of SPIE 3999, 23 (2000); Proceeding of SPIE 3999, 54 (2000); Proceeding of SPIE 4345, 119 (2001).

The formulations disclosed in the aforementioned publications are incorporated herein by reference. It is understood, that the compounds of the present invention are in particular suitable for use as photolatent acid in all the polymers/copolymers and compositions described in these cited publications.

The compounds of the formula I, II and III, and the polymers comprising repeating units derived from a compound of the formula I, II and/or III according to the present invention are suitable as photolatent acids in the bi-layer resist. This technology requests the use of specific polymers/copolymers. Suitable formulations and the preparation of suitable polymer/copolymers are for example published in Proc. SPIE 4345, 361-370 (2001), Proc. SPIE 4345, 406-416 (2001), JP-A-2002-278073, JP-A-2002-30116, JP-A-2002-30118, JP-A-2002-72477, JP-A-2002-348332, JP-A-2003-207896, JP-A-2002-82437, US2003/65101, US2003/64321.

The compounds of the formula I, II and III, and the polymers comprising repeating units derived from a compound of the formula I, II and/or III according to the present invention are suitable as photolatent acids in the multi-layer resist. This technology requests the use of specific polymers/copolymers. Suitable formulations and the preparation of suitable polymer/copolymers are for example published in JP-A-2003-177540, JP-A-2003-280207, JP-A-2003-149822, JP-A-2003-177544.

In order to make fine hole pattern, thermal flow process or chemical shrink technology, so-called RELACS (resolution enhancement lithography assisted by chemical shrink) process, are applied for chemically amplified resist. The compounds of the formula I, II and III, and the polymers comprising repeating units derived from a compound of the formula I, II and/or III according to the present invention are suitable as photolatent acids in the resists for thermal flow process or RELACS process. These technologies request the use of specific polymers/copolymers. Suitable formulations and the preparation of suitable polymer/copolymers are for example published in JP-A-2003-167357, JP-A-2001-337457, JP-A-2003-66626, US2001/53496, Proceeding of SPIE 5039, 789 (2003), IEDM98, Dig., 333 (1998), Proceeding Silicon Technology 11, 12 (1999).

The compounds of the formula I, II and III, and the polymers comprising repeating units derived from a compound of the formula I, II and/or III according to the present invention are suitable as photolatent acids in the F₂ resist technology, i.e. a technology using F₂ excimer lasers (157 nm) for the imaging step. This technology requests the use of specific polymers/copolymers which have high transparency at 157 nm. Examples of polymer suitable for this application are fluoropolymers described in, for example, Proc. SPIE 3999, 330-334 (2000), Proc. SPIE 3999, 357-364 (2000), Proc. SPIE 4345, 273-284 (2001), Proc. SPIE 4345, 285-295 (2001), Proc. SPIE 4345, 296-307 (2001), Proc. SPIE 4345, 327-334 (2001), Proc. SPIE 4345, 350-360 (2001), Proc. SPIE 4345, 379-384 (2001), Proc. SPIE 4345, 385-395 (2001), Proc. SPIE 4345, 417-427 (2001), Proc. SPIE 4345, 428-438 (2001), Proc. SPIE 4345, 439-447 (2001), Proc. SPIE 4345, 1048-1055 (2001), Proc. SPIE 4345, 1066-1072 (2001), Proc. SPIE 4690, 191-199 (2002), Proc. SPIE 4690, 200-211 (2002), Proc. SPIE 4690, 486-496 (2002), Proc. SPIE 4690, 497-503 (2002), Proc. SPIE 4690, 504-511 (2002), Proc. SPIE 4690, 522-532 (2002), US 20020031718, US 20020051938, US 20020055060, US 20020058199, US 20020102490, US 20020146639, US 20030003379, US 20030017404, WO 2002021212, WO 2002073316, WO 2003006413, JP-A-2001-296662, JP-A-2001-350263, JP-A-2001-350264, JP-A-2001-350265, JP-A-2001-356480, JP-A-2002-60475, JP-A-2002-90996, JP-A-2002-90997, JP-A-2002-155112, JP-A-2002-155118, JP-A-2002-155119, JP-A-2002-303982, JP-A-2002-327013, JP-A-2002-363222, JP-A-2003-2925, JP-A-2003-15301, JP-A-2003-2925, JP-A-2003-177539, JP-A-2003-192735, JP-A-2002-155115, JP-A-2003-241386, JP-A-2003-255544, US2003/36016, US2002/81499. Other suitable polymer for F₂ resist is silicon-containing polymers described in, for example, Proc. SPIE 3999, 365-374 (2000), Proc. SPIE 3999, 423-430 (2000), Proc. SPIE 4345, 319-326 (2001), US 20020025495, JP-A-2001-296664, JP-A-2002-179795, JP-A-2003-20335, JP-A-2002-278073, JP-A-2002-55456, JP-A-2002-348332. Polymers containing (meth)acrylonitrile monomer unit described in, for example, JP-A-2002-196495 is also suitable for F₂ resist.

The compounds of the formula I, II and III, and the polymers comprising repeating units derived from a compound of the formula I, II and/or III according to the present invention are suitable as photolatent acids in the EUV resist, i.e. a technology using light source of extreme ultra violet (13 nm) for the imaging step. This technology requests the use of specific polymers/copolymers. Suitable formulations and the preparation of suitable polymer/copolymers are for example published in JP-A-2002-55452, JP-A-2003-177537, JP-A-2003-280199, JP-A-2002-323758, US2002/51932.

The compounds of the formula I, II and III, and the polymers comprising repeating units derived from a compound of the formula I, II and/or III according to the present invention are suitable as photolatent acids in the EB (electron beam) or X-ray resist, i.e. a technology using EB or X-ray for the imaging step. These technologies request the use of specific polymers/copolymers. Suitable formulations and the preparation of suitable polymer/copolymers are for example published in JP-A-2002-99088, JP-A-2002-99089, JP-A-2002-99090, JP-A-2002-244297, JP-A-2003-5355, JP-A-2003-5356, JP-A-2003-162051, JP-A-2002-278068, JP-A-2002-333713, JP-A-2002-31892.

The compounds of the formula I, II and III, and the polymers comprising repeating units derived from a compound of the formula I, II and/or III according to the present invention are suitable as photolatent acids in the chemically amplified resist for immersion lithography. This technology reduces minimum feature size of resist pattern using liquid medium between the light source and the resist as described in Proceeding of SPIE 5040, 667 (2003), Proceeding of SPIE 5040, 679 (2003), Proceeding of SPIE 5040, 690 (2003), Proceeding of SPIE 5040, 724 (2003).

The compounds of the formula I, II and III, and the polymers comprising repeating units derived from a compound of the formula I, II and/or III according to the present invention are suitable as photolatent acids in the positive and negative photosensitive polyimide. This technology requests the use of specific polymers/copolymers. Suitable formulations and the preparation of suitable polymer/copolymers are for example published in JP-A-9-127697, JP-A-10-307393, JP-A-10-228110, JP-A-10-186664, JP-A-11-338154, JP-A-11-315141, JP-A-11-202489, JP-A-11-153866, JP-A-11-84653, JP-A-2000-241974, JP-A-2000-221681, JP-A-2000-34348, JP-A-2000-34347, JP-A-2000-34346, JP-A-2000-26603, JP-A-2001-290270, JP-A-2001-281440, JP-A-2001-264980, JP-A-2001-255657, JP-A-2001-214056, JP-A-2001-214055, JP-A-2001-166484, JP-A-2001-147533, JP-A-2001-125267, JP-A-2001-83704, JP-A-2001-66781, JP-A-2001-56559, JP-A-2001-33963, JP-A-2002-356555, JP-A-2002-356554, JP-A-2002-303977, JP-A-2002-284875, JP-A-2002-268221, JP-A-2002-162743, JP-A-2002-122993, JP-A-2002-99084, JP-A-2002-40658, JP-A-2002-37885, JP-A-2003-26919.

The formulations disclosed in the aforementioned publications are incorporated herein by reference. It is understood, that the compounds of the present invention are in particular suitable for use as photolatent acid in all the polymers/copolymers and compositions described in these cited publications.

Acid-sensitive components that produce a negative resist characteristically are especially compounds which, when catalysed by an acid (e.g. the acid formed during irradiation of the compounds of formulae I, II or III, or the polymers comprising repeating units derived from a compound of the formula I, II and/or III are capable of undergoing a crosslinking reaction with themselves and/or with one or more further components of the composition. Compounds of this type are, for example, the known acid-curable resins, such as, for example, acrylic, polyester, alkyd, melamine, urea, epoxy and phenolic resins or mixtures thereof. Amino resins, phenolic resins and epoxy resins are very suitable. Acid-curable resins of this type are generally known and are described, for example, in “Ullmann's Encyclopädie der technischen Chemie” [Ullmanns Enceclopedia of Technical Chemistry], 4th Edition, Vol. 15 (1978), p. 613-628. The crosslinker components should generally be present in a concentration of from 2 to 40, preferably from 5 to 30, percent by weight, based on the total solids content of the negative resist composition.

Subject of the invention also is a chemically amplified negative photoresist composition.

The invention also pertains to a chemically amplified negative photoresist composition, comprising

(a5) a component which, when catalysed by an acid undergoes a crosslinking reaction with itself and/or with the other components; and
(b) as photosensitive acid donor, at least one compound of the formula I, II and/or III and/or a polymer comprising at least one repeating unit derived from a compound of the formula I, II and/or III and optionally repeating units derived from ethylenically unsaturated compounds selected from the group of formula V.

The invention includes, as a special embodiment, chemically amplified negative, alkali-developable photoresists, comprising

(a4) an alkali-soluble resin as binder
(a5) a component that when catalysed by an acid undergoes a crosslinking reaction with itself and/or with the binder, and
(b) as photosensitive acid donor at least one compound of the formula I, II and/or III and/or polymer comprising at least one repeating unit derived from a compound of the formula I, II and/or III and optionally repeating units derived from ethylenically unsaturated compounds selected from the group of formula V.

The composition may comprise additionally to the component (b) other photosensitive acid donors (b1), other photoinitiators (d) and/or (c) other additives.

Especially preferred as acid-curable resins (a5) are amino resins, such as non-etherified or etherified melamine, urea, guanidine or biuret resins, especially methylated melamine resins or butylated melamine resins, corresponding glycolurils and urones. By “resins” in this context, there are to be understood both customary technical mixtures, which generally also comprise oligomers, and pure and high purity compounds. N-hexa(methoxymethyl) melamine and tetramethoxymethyl glucoril and N,N′-dimethoxymethylurone are the acid-curable resins given the greatest preference.

The concentration of the compound of formula I, II or III in negative resists in general is from 0.1 to 30, preferably up to 20, percent by weight, based on the total solids content of the compositions. From 1 to 15 percent by weight is especially preferred.

Where appropriate, the negative compositions may comprise a film-forming polymeric binder

(a4). This binder is preferably an alkali-soluble phenolic resin. Well suited for this purpose are, for example, novolaks, derived from an aldehyde, for example acetaldehyde or furfuraldehyde, but especially from formaldehyde, and a phenol, for example unsubstituted phenol, mono- or di-chlorosubstituted phenol, such as p-chlorophenol, phenol mono- or di-substituted by C₁-C₉alkyl, such as o-, m- or p-cresol, the various xylenols, p-tert-butylphenol, p-nonylphenol, p-phenylphenol, resorcinol, bis(4-hydroxyphenyl)methane or 2,2-bis(4-hydroxyphenyl)propane. Also suitable are homo- and co-polymers based on ethylenically unsaturated phenols, for example homopolymers of vinyl- and 1-propenyl-substituted phenols, such as p-vinylphenol or p-(1-propenyl)phenol or copolymers of these phenols with one or more ethylenically unsaturated materials, for example styrenes. The amount of binder should generally be from 30 to 95 percent by weight or, preferably, from 40 to 80 percent by weight.

Sulfonate derivatives can also be used as acid, generators, which can be activated photochemically, for the acid-catalysed crosslinking of, for example, poly(glycidyl)methacrylates in negative resist systems. Such crosslinking reactions are described, for example, by Chae at al. in Pollimo 1993, 17(3), 292.

Suitable formulations and the preparation of suitable polymer/copolymers for the negative resist using the compounds of the formula I, II and III, and the polymers comprising repeating units derived from a compound of the formula I, II and/or III according to the present invention are for example published in JP-A-2003-43688, JP-A-2003-114531, JP-A-2002-287359, JP-A-2001-255656, JP-A-2001-305727, JP-A-2003-233185, JP-A-2003-186195, U.S. Pat. No. 6,576,394.

The positive and the negative resist compositions may comprise in addition to the photosensitive acid donor compound of formula I, II or III, or the polymers comprising repeating units derived from a compound of the formula I, II and/or III further photosensitive acid donor compounds (b1), further additives (c), other photoinitiators (d), and/or sensitizers (e). Therefore, subject of the invention also are chemically amplified resist compositions as described above, in addition to components (a) and (b), or components (al), (a2), (a3) and (b), or components (a4), (a5) and (b) comprising further additives (c), further photosensitive acid donor compounds (b1), other photoinitiators (d), and/or sensitizers (e).

Sulfonate derivatives of the present invention in the positive and negative resist can also be used together with other, known photolatent acids (b1), for example, onium salts, 6-nitrobenzylsulfonates, bis-sulfonyl diazomethane compounds, cyano group-containing oximesulfonate compounds, etc. Examples of known photolatent acids for chemically amplified resists are described in U.S. Pat. No. 5,731,364, U.S. Pat. No. 5,800,964, EP 704762, U.S. Pat. No. 5,468,589, U.S. Pat. No. 5,558,971, U.S. Pat. No. 5,558,976, U.S. Pat. No. 6,004,724, GB 2348644 and particularly in EP 794457 and EP 795786.

If a mixture of photolatent acids is used in the resist compositions according to the invention, the weight ratio of sulfonate derivatives of formula I, II or III, or the polymers comprising repeating units derived from a compound of the formula I, II and/or III to the other photolatent acid (b1) in the mixture is preferably from 1:99 to 99:1.

Examples of photolatent acids which are suitable to be used in admixture with the compounds of formula I, II or III, or the polymers comprising repeating units derived from a compound of the formula I, II and/or III are

(1) onium salt compounds, for example,
iodonium salts, sulfonium salts, phosphonium salts, diazonium salts, pyridinium salts. Preferred are diphenyliodonium triflate, diphenyliodonium pyrenesulfonate, diphenyliodonium dodecylbenzenesulfonate, triphenylsulfonium triflate, triphenylsulfonium hexafluoroantimonate, diphenyliodonium hexafluoroantimonate, triphenylsulfonium naphthalenesulfonate, (hydroxyphenyl)benzylmethylsulfonium toluenesulfonate, bis(4-tert-butylphenyl)iodonium bis(nonafluorobutanesulfonyl)imide, bis(4-tert-butylphenyl)iodonium tris(trifluoromethanesulfonyl)methide, triphenylsulfonium bis(trifluoromethanesulfonyl)imide, triphenylsulfonium (octafluorobutane-1,4-disulfonyl)imide, triphenylsulfonium tris(trifluoromethanesulfonyl)methide and the like; the iodonium cation may also be 4-Methylphenyl-4′-isobutylphenyliodonium or 4-Methylphenyl-4′-isopropylphenyliodonium. Particularly preferred are triphenylsulfonium triflate, diphenyliodonium hexafluoroantimonate. Other examples are described in JP-A-2002-229192, JP-A-2003-140332, JP-A-2002-128755, JP-A-2003-35948, JP-A-2003-149800, JP-A-2002-6480, JP-A-2002-116546, JP-A-2002-156750, U.S. Pat. No. 6,458,506, US2003/27061, U.S. Pat. No. 5,554,664.
(2) halogen-containing compounds
haloalkyl group-containing heterocyclic compounds, haloalkyl group-containing hydrocarbon compounds and the like. Preferred are (trichloromethyl)-s-triazine derivatives such as phenyl-bis(trichloromethyl)-s-triazine, methoxyphenyl-bis(trichloromethyl)-s-triazine, naphthyl-bis(trichloromethyl)-s-triazine and the like; 1.1-bis(4-chlorophnyl)-2,2,2-trichloroethane; and the like.
(3) sulfone compounds, for example of the formula

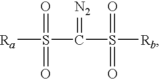

wherein Rₐ and R_{b} independently of one another are alkyl, cycloalkyl or aryl, each of which may have at least one substituent, e.g.

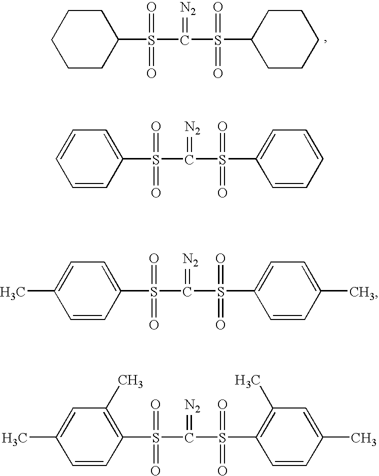

Such compounds are disclosed for example in US 2002/0172886-A, JP-A-2003-192665, US2002/9663. More examples are β-ketosulfones, β-sulfonylsulfones and their α-diazo derivatives and the like. Preferred are phenacylphenylsulfone, mesitylphenacylsulfone, bis(phenylsulfonyl)methane, bis(phenylsulfonyl)diazomethane.
(4) sulfonate compounds, for example
alkylsulfonic acid esters, haloalkylsulfonic acid esters, arylsulfonic acid esters, iminosulfonates, imidosulfonates and the like. Preferred imidosulfonate compounds are, for example, N-(trifluoromethlsulfonyloxy)succinimide, N-(trifluoromethylsulfonyloxy)phthalimide, N-(trifluoromethylsulfonyloxy)naphthylimide, N-(trifluoromethylsulfonyloxy)diphenylmaleimide, N-(trifluoromethylsulfonyloxy)-bicyclo-[2,2,1]-hept-5-ene-2,3-dicarboximide, N-(trifluoromethylsulfonyloxy)-7-oxabicyclo-[2,2,1]-hept-5-ene-2,3-dicarboximide, N-(trifluoromethylsulfonyloxy)-7-oxabicyclo-[2,2,1]-hept-5-ene-2,3-dicarboximide, N-(trifluoromethylsulfonyloxy)-bicyclo-[2,2,1]-heptan-5,6-oxy-2,3-dicarboximide, N-(camphanylsulfonyloxy) succinimide, N-(camphanylsulfonyloxy)phthalimide, N-(camphanylsulfonyloxy)naphthylimide, N-(camphanylsulfonyloxy)diphenylmaleimide, N-(camphanylsulfonyloxy)bicyclo-[2,2,1]-hept-5-ene-2,3-dicarboximide, N-(camphanylsulfonyloxy)-7-oxabicyclo-[2,2,1]-hept-5-ene-2,3-dicarboximide, N-(camphanylsulfonyloxy)-7-oxabicyclo-[2,2,1]hept-5-ene-2,3-dicarboximide, N-(camphanylsulfonyloxy)-bicyclo-[2,2,1]-heptan-5,6-oxy-2,3-dicarboximide, N-(4-methylphenylsulfonyloxy)succinimide, N-(4-methylphenylsulfonyloxy)phthalimide, N-(4-methylphenylsulfonyloxy)naphthylimide, N-(4-methylphenylsulfonyloxy)naphthylimide, N-(4-methylphenylsulfonyloxy)diphenylmaleimide, N-(4-methylphenylsulfonyloxy)-bicyclo-[2,2,1]-hept-5-ene-2:3-dicarboximide, N-(4-methylphenylsulfonyloxy)-7-oxabicyclo-[2,2,1]-hept-5-ene-2,3-dicarboximide, N-(4-methylphenylsulfonyloxy)-bicyclo-[2,2,1]-heptan-5,6-oxy-2,3-dicarboximide, N-(2-trifluoromethylphenylsulfonyloxy)succinimide, N-(2-trifluoromethylphenylsulfonyloxy)naphthylimide, N-(2-trifluoromethylphenylsulfonyloxy)diphenylmaleimide, N-(2-trifluoromethylphenylsulfonyloxy)-bicyclo-[2,2,1]-hept-5-ene-2,3-dicarboximide, N-(2-trifluoromethylphenylsulfonyloxy)-7-oxabicyclo-[2,2,1]-hept-5-ene-2,3-dicarboximide, N-(2-trifluoromethylphenylsulfonyloxy)-bicyclo-[2,2,1]-heptan-5,6-oxy-2,3-dicarboximide and the like.

Other suitable sulfonate compounds preferably are, for example, benzoin tosylate, pyrogallol tristriflate, pyrogallolomethanesulfonic acid triester, nitorobenzyl-9,10-diethyoxyanthracene-2-sulfonate, α-(4-toluene-sulfonyloxyimino)-benzyl cyanide, α-(4-toluene-sulfonyloxyimino)-4-methoxybenzyl cyanide, α-(4-toluene-sulfonyloxyimino)-2-thienylmethyl cyanide, α-(methanesulfonyloxyimino)-1-cyclohexenylacetonitrile, α-(butylsulfonyloxyimino)-1-cyclopentenylacetonitrile, (4-methylsulfonyloxyimino-cyclohexa-2,5-dienylidene)-phenyl-acetonitrile, (5-methylsulfonyloxyimino-5H-thiophen-2-ylidene)-phenyl-acetonitrile, (5-methylsulfonyloxyimino-5H-thiophen-2-ylidene)-(2-methylphenyl)-acetonitrile, (5-propylsulfonyloxyimino-5H-thiophen-2-ylidene)-(2-methylphenyl)-acetonitrile, (5-(p-toluenesulfonyloxyimino)-5H-thiophen-2-ylidene)-(2-methylphenyl)-acetonitrile, (5-(10-camphorsulfonyloxyimino)-5H-thiophen-2-ylidene)-(2-methylphenyl)-acetonitrile, (5-methylsulfonyloxyimino-5H-thiophen-2-ylidene)-(2-chlorophenyl)-acetonitrile, 2,2,2-trifluoro-1-{4-(3-[4-{2,2,2-trifluoro-1-(1-propanesulfonyloxyimino)-ethyl}-phenoxy]-propo xy)-phenyl}-ethanone oxime 1-propanesulfonate, 2,2,2-trifluoro-1-{4-(3-[4-{2,2,2-trifluoro-1-(1-p-toluenesulfonyloxyimino)-ethyl}-phenoxy]-prop oxy)-phenyl}-ethanone oxime 1-p-toluenesulfonate, 2-[2,2,3,3,4,4,5,5,6,6,7,7-dodecafluoro-1-(nonafluorobutylsulfonyloxyimino)-heptyl]-fluorene, 2-[2,2,3,3,4,4,4-heptafluoro-1-(nonafluorobutylsulfonyloxyimino)-butyl]fluorene, 2-[2,2,3,3,4,4,5,5-octafluoro-1-(nonafluorobutylsulfonyloxyimino)-pentyl]fluorene and the like.

In the radiation sensitive resin composition of this invention, particularly preferred sulfonate compounds include pyrogallolmethanesulfonic acid triester, N-(trifluoromethylsulfonyloxy)bicyclo-[2,2,1]-hept-5-ene-2,3-dicarboximide, N-(camphanylsulfonyloxy)naphthylimide, N-(2-trifluoromethylphenylsulfonyloxy)phthalimide, N-(trifluoromethylsulfonyloxy)-bicyclo-[2,2,1]-hept-5-ene-2,3-dicarboximide, N-(camphanylsulfonyloxy)naphthylimide, N-(2-trifluoromethylphenylsulfonyloxy)phthalimide and the like.

(5) Quinonediazide compounds, for example
1,2-quinonediazidesulfonic acid ester compounds of polyhydroxy compounds. Preferred are compounds having a 1,2-quinonediazidesulfonyl group, e.g. a 1,2-benzoquinonediazide-4-sulfonyl group, a 1,2-naphthoquinonediazide-4-sulfonyl group, a 1,2-naphthoquinonediazide-5-sulfonyl group, a 1,2-naphthoquinonediazide-6-sulfonyl group or the like. Particularly preferred are compounds having a 1,2-naphthoquinonediazide-4-sulfonyl group or a 1,2-naphthoquinonediazide-5-sulfonyl group. In particular suitable are 1,2-quinonediazidesulfonic acid esters of (poly)hydroxyphenyl aryl ketones such as 2,3,4-trihydroxybenzophenone, 2,4,6-trihydroxybenzophenone, 2,3,4,4′-tetrahydroxybenzophenone, 2,2′,3,4-tetrahydroxybenzophenone, 2,3,4,4′-tetrahydroxybenzophenone, 2,2′,4,4′-tetrahydroxybenzophenone 2,2′,3,4,4′-pentahydroxybenzophenone, 2,2′3,2,6′-pentahydroxybenzophenone, 2,3,3′,4,4′5′-hexahydroxybenzophenone, 2,3′,4,4′,5′6-hexahydroxybenzophenone and the like; 1,2-quinonediazidesulfonic acid esters of bis-[(poly)hydroxyphenyl]alkanes such as bis(4-hydroxyphenyl)ethane, bis(2,4-dihydroxyphenyl)ethane, 2,2-bis(4-hydroxyphenyl)propane, 2,2-bis(2,4-dihydroxyphenyl)propane, 2,2-bis-(2,3,4-tridroxyphenyl)propane and the like; 1,2-quinonediazidesulfonic acid esters of (poly)hydroxyphenylalkanes such as 4,4′-dihydroxytriphenylmethane, 4,4′4″-trihydroxytriphenylmethane, 4,4′5,5′-tetramethyl-2,2′2″-trihydroxytriphenylmethane, 2,2,5,5′-tetramethyl-4,4′,4″-trihydroxytriphenylmethane, 1,1,1-tris(4-hydroxyphenyl)ethane, 1,1-bis(4-hydroxyphenyl)-1-phenylethane, 1,1-bis(4-hydroxyphenyl)-1-(4-[1-(hydroxyphenyl)-1-methylethyl]phenyl)ethane and the like; 1,2-quinonediazidesulfonic acid esters of (poly)hydroxyphenylflavans such as 2,4,4-trimethyl-2′,4′,7-trihydroxy-2-phenylflavan, 2,4,4-trimethyl-2′,4′,5′,6,7-pentahydroxy-2-phenylflavan and the like.

Other examples of photolatent acids which are suitable to be used in admixture with the compounds according to the present invention are described in JP-A-2003-43678, JP-A-2003-5372, JP-A-2003-43677, JP-A-2002-357904, JP-A-2002-229192.

The positive and negative photoresist composition of the present invention may optionally contain one or more additives (c) customarily used in photoresists in the customary amounts known to a person skilled in the art, for example, dyes, pigments, plasticizers, surfactants, flow improvers, wetting agents, adhesion promoters, thixotropic agents, colourants, fillers, solubility accelerators, acid-amplifier, photosensitizers and organic basic compounds. Further examples for organic basic compounds which can be used in the resist composition of the present invention are compounds which are stronger bases than phenol, in particular, nitrogen-containing basic compounds. These compounds may be ionic, like, for example, tetraalkylammonium salts or non-ionic. Preferred organic basic compounds are nitrogen-containing basic compounds having, per molecule, two or more nitrogen atoms having different chemical environments. Especially preferred are compounds containing both at least one substituted or unsubstituted amino group and at least one nitrogen-containing ring structure, and compounds having at least one alkylamino group. Examples of such preferred compounds include guanidine, aminopyridine, amino alkylpyridines, aminopyrrolidine, indazole, imidazole, pyrazole, pyrazine, pyrimidine, purine, imidazoline, pyrazoline, piperazine, aminomorpholine, and aminoalkylmorpholines. Suitable are both, the unsubstituted compounds or substituted derivatives thereof. Preferred substituents include amino, aminoalkyl groups, alkylamino groups, aminoaryl groups, arylamino groups, alkyl groups alkoxy groups, acyl groups acyloxy groups aryl groups, aryloxy groups, nitro, hydroxy, and cyano. Specific examples of especially preferred organic basic compounds include guanidine, 1,1-dimethylguanidine, 1,1,3,3-tetramethylguanidine, 2-aminopyridine, 3-aminopyridine, 4-aminopyridine, 2-dimethylaminopyridine, 4-dimethylaminopyridine, 2-diethylaminopyridine, 2-(aminomethyl)pyridine, 2-amino-3-methylpyridine, 2-amino-4-methylpyridine, 2-amino-5-methylpyridine, 2-amino-6-methylpyridine, 3-aminoethylpyridine, 4-aminoethylpyridine, 3-aminopyrrolidine, piperazine, N-(2-aminoethyl)piperazine, N-(2-aminoethyl)piperidine, 4-amino-2,2,6,6-tetramethylpiperidine, 4-piperidinopiperidine, 2-imimopiperidine, 1-(2-aminoethyl)pyrrolidine, pyrazole, 3-amino-5-methylpyrazole, 5-amino-3-methyl-1-p-tolylpyrazole, pyrazine, 2-(aminomethyl)-5-methylpyrazine, pyrimidine, 2,4-diaminopyrimidine, 4,6-dihydroxypyrimidine, 2-pyrazoline, 3-pyrazoline, N-aminomorpholine, and N-(2-aminoethyl)morpholine.

Other examples of suitable organic basic compounds are described in DE 4408318, U.S. Pat. No. 5,609,989, U.S. Pat. No. 5,556,734, EP 762207, DE 4306069, EP 611998, EP 813113, EP 611998, and U.S. Pat. No. 5,498,506, JP-A-2003-43677, JP-A-2003-43678, JP-A-2002-226470, JP-A-2002-363146, JP-A-2002-363148, JP-A-2002-363152, JP-A-2003-98672, JP-A-2003-122013, JP-A-2002-341522. However, the organic basic compounds suitable in the present invention are not limited to these examples.

The nitrogen-containing basic compounds may be used alone or in combination of two or more thereof. The added amount of the nitrogen-containing basic compounds is usually from 0.001 to 10 parts by weight, preferably from 0.01 to 5 parts by weight, per 100 parts by weight of the photosensitive resin composition (excluding the solvent). If the amount thereof is smaller than 0.001 part by weight, the effects of the present invention cannot be obtained. On the other hand, if it exceeds 10 parts by weight, reduced sensitivity and impaired developability at unexposed parts are liable to be caused.

The composition can further contain a basic organic compound which decomposes under actinic radiation (“suicide base”) such as for example described in EP 710885, U.S. Pat. No. 5,663,035, U.S. Pat. No. 5,595,855, U.S. Pat. No. 5,525,453, and EP 611998.

Examples of dyes (c) suitable for the compositions of the present invention are oil-soluble dyes and basic dyes, e.g. Oil Yellow #101, Oil Yellow #103, Oil Pink #312, Oil Green BG, Oil Blue BOS, Oil Blue #603, Oil Black BY, Oil Black BS, Oil Black T-505 (all manufactured by Orient Chemical Industries Ltd., Japan), crystal violet (CI42555), methyl violet (CI 42535), rhodamine B (CI 45170B), malachite green (CI 42000), and methylene blue (CI52015).

Spectral sensitizers (e) may be further added to sensitize the photo latent acid to exhibit absorption in a region of longer wavelengths than far ultaviolet, whereby the photosensitive composition of the present invention can, for example, be rendered sensitive to an i-line or g-line radiation. Examples of suitable spectral sensitizers include benzophenones, p,p′-tetramethyldiaminobenzophenone, p,p′-tetraethylethylaminobenzophenone, thioxanthone, 2-chlorothioxanthone, anthrone, pyrene, perylene, phenothiazine, benzil, acridine orange, benzoflavin, cetoflavin T, 9,10-diphenylanthracene, 9-fluorenone, acetophenone, phenanthrene, 2-nitrofluorene, 5-nitroacenaphthene, benzoquinone, 2-chloro-4-nitroaniline, N-acetyl-p-nitroaniline, p-nitroaniline, N-acetyl-4-nitro-1-naphthylamine, picramide, anthraquinone, 2-ethylanthraquinone, 2-tert-butylanthraquinone, 1,2-benzanthraquinone, 3-methyl-1,3-diaza-1,9-benzanthrone, dibenzalacetone, 1,2-naphthoquinone, 3-acylcoumarin derivatives, 3,3′-carbonyl-bis(5,7-dimethoxycarbonylcoumarin), 3-(aroylmethylene) thiazolines, eosin, rhodamine, erythrosine, and coronene. However, the suitable spectral sensitizers are not limited to these examples.

These spectral sensitizers can be used also as light absorbers for absorbing the far ultraviolet emitted by a light source. In this case, the light absorber reduces light reflection from the substrate and lessens the influence of multiple reflection within the resist film, thereby diminishing the effect of standing waves.

Specific examples of such compounds are

### 1. Thioxanthones

Thioxanthone, 2-isopropylthioxanthone, 2-chlorothioxanthone, 1-chloro-4-propoxythioxanthone, 2-dodecylthioxanthone, 2,4-diethylthioxanthone, 2,4-dimethylthioxanthone, 1-methoxycarbonylthioxanthone, 2-ethoxycarbonylthioxanthone, 3-(2-methoxyethoxycarbonyl)-thioxanthone, 4-butoxycarbonylthioxanthone, 3-butoxycarbonyl-7-methylthioxanthone, 1-cyano-3-chlorothioxanthone, 1-ethoxycarbonyl-3-chlorothioxanthone, 1-ethoxycarbonyl-3-ethoxythioxanthone, 1-ethoxycarbonyl-3-aminothioxanthone, 1-ethoxycarbonyl-3-phenylsulfurylthioxanthone, 3,4-di-[2-(2-methoxyethoxy)ethoxycarbonyl]-thioxanthone, 1,3-dimethyl-2-hydroxy-9H-thioxanthen-9-one 2-ethylhexylether, 1-ethoxycarbonyl-3-(1-methyl-1-morpholinoethyl)-thioxanthone, 2-methyl-6-dimethoxymethyl-thioxanthone, 2-methyl-6-(1,1-dimethoxybenzyl)-thioxanthone, 2-morpholinomethylthioxanthone, 2-methyl-6-morpholinomethylthioxanthone, N-allylthioxanthone-3,4-dicarboximide, N-octylthioxanthone-3,4-dicarboximide, N-(1,1,3,3-tetramethylbutyl)-thioxanthone-3,4-dicarboximide, 1-phenoxythioxanthone, 6-ethoxycarbonyl-2-methoxythioxanthone, 6-ethoxycarbonyl-2-methylthioxanthone, thioxanthone-2-carboxylic acid polyethyleneglycol ester, 2-hydroxy-3-(3,4-dimethyl-9-oxo-9H-thioxanthon-2-yloxy)-N,N,N-trimethyl-1-propanaminium chloride;

### 2. Benzophenones

benzophenone, 4-phenyl benzophenone, 4-methoxy benzophenone, 4,4′-dimethoxy benzophenone, 4,4′-dimethyl benzophenone, 4,4′-dichlorobenzophenone 4,4′-bis(dimethylamino)benzophenone, 4,4′-bis(diethylamino)benzophenone, 4,4′-bis(methylethylamino)benzophenone, 4,4′-bis(p-isopropylphenoxy)benzophenone, 4-methyl benzophenone, 2,4,6-trimethylbenzophenone, 3-methyl-4′-phenyl-benzophenone, 2,4,6-trimethyl-4′-phenyl-benzophenone, 4-(4-methylthiophenyl)-benzophenone, 3,3′-dimethyl-4-methoxy benzophenone, methyl-2-benzoylbenzoate, 4-(2-hydroxyethylthio)-benzophenone, 4-(4-tolylthio)benzophenone, 1-[4-(4-benzoyl-phenylsulfanyl)-phenyl]-2-methyl-2-(toluene-4-sulfonyl)-propan-1-one, 4-benzoyl-N,N,N-trimethylbenzenemethanaminium chloride, 2-hydroxy-3-(4-benzoylphenoxy)-N,N,N-trimethyl-1-propanaminium chloride monohydrate, 4-(13-acryloyl-1,4,7,10,13-pentaoxamidecyl)-benzophenone, 4-benzoyl-N,N-dimethyl-N-[2-(1-oxo-2-propenyl)oxy]ethyl-benzenemethanaminium chloride;

### 3. Coumarins

Coumarin 1, Coumarin 2, Coumarin 6, Coumarin 7, Coumarin 30, Coumarin 102, Coumarin 106, Coumarin 138, Coumarin 152, Coumarin 153, Coumarin 307, Coumarin 314, Coumarin 314T, Coumarin 334, Coumarin 337, Coumarin 500, 3-benzoyl coumarin, 3-benzoyl-7-methoxycoumarin, 3-benzoyl-5,7-dimethoxycoumarin, 3-benzoyl-5,7-dipropoxycoumarin, 3-benzoyl-6,8-dichlorocoumarin, 3-benzoyl-6-chloro-coumarin, 3,3′-carbonyl-bis-[5,7-di(propoxy)-coumarin], 3,3′-carbonyl-bis(7-methoxycoumarin), 3,3′-carbonyl-bis(7-diethylamino-coumarin), 3-isobutyroylcoumarin, 3-benzoyl-5,7-dimethoxy-coumarin, 3-benzoyl-5,7-diethoxy-coumarin, 3-benzoyl-5,7-dibutoxycoumarin, 3-benzoyl-5,7-di(methoxyethoxy)-coumarin, 3-benzoyl-5,7-di(allyloxy)coumarin, 3-benzoyl-7-dimethylaminocoumarin, 3-benzoyl-7-diethylaminocoumarin, 3-isobutyroyl-7-dimethylaminocoumarin, 5,7-dimethoxy-3-(1-naphthoyl)-coumarin, 5,7-diethoxy-3-(1-naphthoyl)-coumarin, 3-benzoylbenzo[f]coumarin, 7-diethylamino-3-thienoylcoumarin, 3-(4-cyanobenzoyl)-5,7-dimethoxycoumarin, 3-(4-cyanobenzoyl)-5,7-dipropoxycoumarin, 7-dimethylamino-3-phenylcoumarin, 7-diethylamino-3-phenylcoumarin, the coumarin derivatives disclosed in JP 09-179299-A and JP 09-325209-A, for example 7-[{4-chloro-6-(diethylamino)-S-triazine-2-yl}amino]-3-phenylcoumarin;
4. 3-(aroylmethylene)-thiazolines
3-methyl-2-benzoylmethylen e-β-naphthothiazoline, 3-methyl-2-benzoylmethylene-benzothiazoline, 3-ethyl-2-propionylmethylene-β-naphthothiazoline;

### 5. Rhodanines

4-dimethylaminobenzalrhodanine, 4-diethylaminobenzalrhodanine, 3-ethyl-5-(3-octyl-2-benzothiazolinylidene)-rhodanine, the rhodanine derivatives, formulae [1], [2], [7], disclosed in JP 08-305019A;
6. Other compounds
acetophenone, 3-methoxyacetophenone, 4-phenylacetophenone, benzil, 4,4′-bis(dimethylamino)benzil, 2-acetylnaphthalene, 2-naphthaldehyde, dansyl acid derivatives, 9,10-anthraquinone, anthracene, pyrene, aminopyrene, perylene, phenanthrene, phenanthrenequinone, 9-fluorenone, dibenzosuberone, curcumin, xanthone, thiomichler's ketone, α-(4-dimethylaminobenzylidene) ketones, e.g. 2,5-bis(4-diethylaminobenzylidene)cyclopentanone, 2-(4-dimethylamino-benzylidene)-indan-1-one, 3-(4-dimethylamino-phenyl)-1-indan-5-yl-propenone, 3-phenylthiophthalimide, N-methyl-3,5-di(ethylthio)-phthalimide, N-methyl-3,5-di(ethylthio)-phthalimide, phenothiazine, methylphenothiazine, amines, e.g. N-phenylglycine, ethyl 4-dimethylaminobenzoate, butoxyethyl 4-dimethylaminobenzoate, 4-dimethylaminoacetophenone, triethanolamine, methyldiethanolamine, dimethylaminoethanol, 2-(dimethylamino)ethyl benzoate, poly(propylenegylcol)-4-(dimethylamino) benzoate, pyrromethenes, e.g., 1,3,5,7,9-pentamethylpyrromethene BF₂ complex, 2,8-diethyl-1,3,5,7,9-pentamethylpyrromethene BF₂ complex, 2,8-diethyl-5-phenyl-1,3,7,9-tetramethylpyrromethene BF₂ complex, 9,10-bis(phenylethynyl)-1,8-dimethoxyanthracene, benzo[1,2,3-kl:4,5,6-k′l′]dixanthene.

Further suitable additives (c) are “acid-amplifiers”, compounds that accelerate the acid formation or enhance the acid concentration. Such compounds may also be used in combination with the sulfonate derivatives of the formulae I, II or III, or the polymers comprising repeating units derived from a compound of the formula I, II and/or III according to the invention in positive or negative resists, or in imaging systems as well as in all coating applications. Such acid amplifiers are described e.g. in Arimitsu, K. et al. J. Photopolym. Sci. Technol. 1995, 8, pp 43; Kudo, K. et al. J. Photopolym. Sci. Technol. 1995, 8, pp 45; Ichimura, K. et al. Chem: Letters 1995, pp 551.

Other additives (c) to improve the resist performance such as resolution, pattern profile, process latitude, line edge roughness, stability are described in JP-A-2002-122992, JP-A-2002-303986, JP-A-2002-278071, JP-A-2003-57827, JP-A-2003-140348, JP-A-2002-6495, JP-A-2002-23374, JP-A-2002-90987, JP-A-2002-91004, JP-A-2002-131913, JP-A-2002-131916, JP-A-2002-214768, JP-A-2001-318464, JP-A-2001-330947, JP-A-2003-57815, JP-A-2003-280200, JP-A-2002-287362, JP-A-2001-343750. Such compounds may also be used in combination with the sulfonate derivatives of the formulae I, II or III, or the polymers comprising repeating units derived from a compound of the formula I, II and/or III according to the invention in positive or negative resists.

Usually, for the application to a substrate of the photosensitive composition of the present invention, the composition is dissolved in an appropriate solvent. Preferred examples of these solvents include ethylene dichloride, cyclohexanone, cyclopentanone, 2-heptanone, γ-butyrolactone, methyl ethyl ketone, ethylene glycol monomethyl ether, ethylene glycol monoethyl ether, 2-methoxyethyl acetate, 2-ethoxyethyl acetate, 2-ethoxyethanol, diethyl glycol dimethyl ether, ethylene glycol monoethyl ether acetate, propylene glycol monomethyl ether, propylene glycol monomethyl ether acetate, toluene, ethyl acetate, butyl acetate, methyl lactate, ethyl lactate, methyl methoxypropionate, ethyl ethoxypropionate, methyl pyruvate, ethyl pyruvate, propyl pyruvate, N,N-dimethylformamide, dimethyl sulfoxide, N-methylpyrrolidone, and tetrahydrofuran. These solvents may be used alone or as mixtures. Preferred examples of the solvents are esters, such as 2-methoxyethyl acetate, ethylene glycolmonoethyl ether acetate, propylene glycol monomethyl ether acetate, methyl methoxypropionate, ethyl ethoxypropionate, and ethyl lactate. Use of such solvents is advantageous because the sulfonate derivatives represented by formulae I, II or III, or the polymers comprising repeating units derived from a compound of the formula I, II and/or III according to the present invention have good compatibility therewith and better solubility therein.

A surfactant can be added to the solvent. Examples of suitable surfactants include nonionic surfactants, such as polyoxyethylene alkyl ethers, e.g. polyoxyethylene lauryl ether, polyoxyethylene stearyl ether, polyoxyethylene acetyl ether, and polyoxyethylene coleyl ether; polyoxyethylene alkylaryl ethers, e.g. polyoxyethylene, octylphenol ether and polyoxyethylene non-ylphenol ether; polyoxyethylene/polyoxypropylene block copolymers, sorbitan/fatty acid esters, e.g. sorbitan monolaurate, sorbitan monopalmitate, sorbitan monostearate, sorbitan monooleate, sorbitan trioleate; fluorochemical surfactants such as F-top EF301, EF303, and EF352 (manufactured by New Akita Chemical Company, Japan). Megafac F171 and F17.3 (manufactured by Dainippon Ink & Chemicals, Inc, Japan), Fluorad FC 430 and FC₄₃₁ (manufactured by Sumitomo 3M Ltd., Japan), Asahi Guard AG710 and Surflon S-382, SC101, SC102, SC103, SC104, SC105, and SC106 (manufactured by Asahi Grass Col, Ltd., Japan); organosiloxane polymer KP341 (manufactured by Shin-Etsu Chemical Co., Ltd., Japan); and acrylic or methacrylic (co)polymers Poly-flow Now.75 and NO.95 (manufactured by Kyoeisha Chemical Co., Ltd., Japan). Other examples are described in JP-A-2001-318459, JP-A-2002-6483. The added amount of the surfactant usually is 2 parts by weight or lower, desirably 0.5 part by weight or lower, per 100 parts by weight of the solid components of the composition of the present invention. The surfactants may be added alone or in combination of two or more thereof.

The solution is uniformly applied to a substrate by means of known coating methods, for example by spin-coating, immersion, knife coating, curtain pouring techniques, brush application, spraying and roller coating. It is also possible to apply the photosensitive layer to a temporary, flexible support and then to coat the final substrate by coating transfer (laminating). The amount applied (coating thickness) and the nature of the substrate (coating substrate) are dependent on the desired field of application. The range of coating thicknesses can in principle include values from approximately 0.01 μm to more than 100 μm.

After the coating operation generally the solvent is removed by heating, resulting in a layer of the photoresist on the substrate. The drying temperature must of course be lower than the temperature at which certain components of the resist might react or decompose. In general, drying temperatures are in the range from 60 to 160° C.

The resist coating is then irradiated image-wise. The expression “image-wise irradiation” includes irradiation in a predetermined pattern using actinic radiation, i.e. both irradiation through a mask containing a predetermined pattern, for example a transparency, a chrome mask or a reticle, and irradiation using a laser beam or electron beam that writes directly onto the resist surface, for example under the control of a computer, and thus produces an image. Another way to produce a pattern is by interference of two beams or images as used for example in holographic applications. It is also possible to use masks made of liquid crystals that can be addressed pixel by pixel to generate digital images, as is, for example described by A. Bertsch; J. Y. Jezequel; J. C. Andre in Journal of Photochemistry and Photobiology A: Chemistry 1997, 107 pp. 275-281 and by K. P. Nicolay in Offset Printing 1997, 6, pp. 34-37.

After the irradiation and, if necessary, thermal treatment, the irradiated sites (in the case of positive resists) or the non-irradiated sites (in the case of negative resists) of the composition are removed in a manner known per se using a developer.

In order to accelerate the catalytic reaction and hence the development of a sufficient difference in solubility between the irradiated and unirradiated sections of the resist coating in the developer, the coating is preferably heated before being developed. The heating can also be carried out or begun during the irradiation. Temperatures of from 60 to 160° C. are preferably used. The period of time depends on the heating method and, if necessary, the optimum period can be determined easily by a person skilled in the art by means of a few routine experiments. It is generally from a few seconds to several minutes. For example, a period of from 10 to 300 seconds is very suitable when a hotplate is used and from 1 to 30 minutes when a convection oven is used. It is important for the latent acid donors according to the invention in the unirradiated sites on the resist to be stable under those processing conditions.

The coating is then developed, the portions of the coating that, after irradiation, are more soluble in the developer being removed. If necessary, slight agitation of the workpiece, gentle brushing of the coating in the developer bath or spray developing can accelerate that process step. The aqueous-alkaline developers customary in resist technology may, for example, be used for the development. Such developers comprise, for example, sodium or potassium hydroxide, the corresponding carbonates, hydrogen carbonates, silicates or metasilicates, but preferably metal-free bases, such as ammonia or amines, for example ethylamine, n-propylamine, diethylamine, di-n-propylamine, triethylamine, methyl diethylamine, alkanolamines, for example dimethyl ethanolamine, triethanolamine, quaternary ammonium hydroxides, for example tetramethylammonium hydroxide or tetraethylammonium hydroxide. The developer solutions are generally up to 0.5 N, but are usually diluted in suitable manner before use. For example solutions having a normality of approximately 0.1-0.3 are well suited. The choice of developer depends on the nature of the photocurable surface coating, especially on the nature of the binder used or of the resulting photolysis products. The aqueous developer solutions may, if necessary, also comprise relatively small amounts of wetting agents and/or organic solvents. Typical organic solvents that can be added to the developer fluids are, for example, cyclohexanone, 2-ethoxyethanol, toluene, acetone, isopropanol and also mixtures of two or more of these solvents. A typical aqueous/organic developer system is based on Butylcellosolve®/water.

Subject of the invention also is a process for the preparation of a photoresist by
(1) applying to a substrate a composition as described above;
(2) post apply baking the composition at temperatures between 60° C. and 160° C.;
(3) image-wise irradiating with light of wavelengths between 10 nm and 1500 nm;
(4) optionally post exposure baking the composition at temperatures between 60° C. and 160° C.; and
(5) developing with a solvent or with an aqueous alkaline developer.

Preferred is a process, wherein the image-wise irradiation is carried out with monochromatic or polychromatic radiation in the wavelength range from 150 to 450 nm, in particular in the range from 190 to 260 nm.

The photoresist compositions can be used on all substrates and with all exposure techniques known to the person skilled in the art. For example, semiconductor substrates can be used, such as silicon, gallium arsenide, germanium, indium antimonide; furthermore substrate covered by oxide or nitride layers, such as silicon dioxide, silicon nitride, titanium nitride, siloxanes, as well as metal substrates and metal coated substrates with metals such as aluminium, copper, tungsten, etc. The substrate can also be coated with polymeric materials, for example with organic antireflective coatings, insulation layers and dielectric coatings from polymeric materials prior to coating with the photoresist.

The photoresist layer can be exposed by all common techniques, such as direct writing, i.e. with a laser beam or projection lithography in step- and repeat mode or scanning mode, or by contact printing through a mask.

In case of projection lithography a wide range of optical conditions can be used such as coherent, partial coherent or incoherent irradiation. This includes off-axis illumination techniques, for example annular illumination and quadrupol illumination where the radiation is allowed to pass only certain regions of the lens, excluding the lens center.

The mask used to replicate the pattern can be a hard mask or a flexible mask. The mask can include transparent, semitransparent and opaque patterns. The pattern size can include also patterns which are at or below the resolution limit of the projection optics and placed on the mask in a certain way in order to modify the aerial image, intensity and phase modulation of the irradiation after having passed the mask. This includes phase shift masks and half-tone phase shift masks.

The patterning process of the photoresist composition can be used to generate patterns of any desired geometry and shape, for example dense and isolated lines, contact holes, trenches, dots, etc.

The photoresists according to the invention have excellent lithographic properties, in particular a high sensitivity, and high resist transparency for the imaging radiation.

Possible areas of use of the composition according to the invention are as follows: use as photoresists for electronics, such as etching resists, ion-implantation resist, electroplating resists or solder resists, the manufacture of integrated circuits or thin film transistor-resist (TFT); the manufacture of printing plates, such as offset printing plates or screen printing stencils, use in the etching of mouldings or in stereolithography or holography techniques, which are employed for various applications, for example, 3D optical information storage described in J. Photochem. Photobio. A, 158, 163 (2003), Chem. Mater. 14, 3656 (2002). The composition according to the invention is also suitable for making inter-metal dielectrics layer, buffer layer, passivation coat of semiconductor devices and suitable for making waveguide for optoelectronics. For MEMS (micro electro mechanical systems) application, the composition according to the invention can be used as etching resist, mold for material deposition, and three dimensional objects of device itself. The coating substrates and processing conditions vary accordingly. Such example is described in U.S. Pat. No. 6,391,523.

The compounds of formula I, II and III, and the polymers comprising repeating units derived from a compound of the formula I, II and/or III according to the present invention, in combination with a sensitizer compound as described above, can also be used in holographic data storage (HDS) systems as for example described in WO 03/021358.

The compositions according to the invention are also outstandingly suitable as coating compositions for substrates of all types, including wood, textiles, paper, ceramics, glass, plastics, such as polyesters, polyethylene terephthalate, polyolefins or cellulose acetate, especially in the form of films, but especially for coating metals, such as Ni, Fe, Zn, Mg, Co or especially Cu and Al, and also Si, silicon oxides or nitrides, to which an image is to be applied by means of image-wise irradiation.

The invention relates also to the use of compounds of formula I, II or III, or the polymers comprising repeating units derived from a compound of the formula I, II and/or III as photolatent acid donors in compositions that can be crosslinked under the action of an acid and/or as dissolution enhancers in compositions wherein the solubility is increased under the action of an acid.

Subject of the invention further is a process of crosslinking compounds that can be crosslinked under the action of an acid, which method comprises adding a compound of formula I, II or III, or the polymers comprising repeating units derived from a compound of the formula I, II and/or III to the above-mentioned compositions and irradiating imagewise or over the whole area with light having a wavelength of 10-1500 nm.

The invention relates also to the use of compounds of formulae I, II or III, or the polymers comprising repeating units derived from a compound of the formula I, II and/or, III as photosensitive acid donors in the preparation of pigmented and non-pigmented surface coatings, adhesives, laminating adhesives, structural adhesives, pressure-sensitive adhesives, printing inks, printing plates, relief printing plates, planographic printing plates, intaglio printing plates, processless printing plates, screen printing stencils, dental compositions, colour filters, spacers, electroluminescence displays and liquid crystal displays (LCD), waveguides, optical switches, color proofing systems, resists, photoresists for electronics, electroplating resists, etch resists both for liquid and dry films, solder resist, photoresist materials for a UV and visible laser direct imaging system, photoresist materials for forming dielectric layers in a sequential build-up layer of a printed circuit board, image-recording materials, image-recording materials for recording holographic images, optical information storage or holographic data storage, decolorizing materials, decolorizing materials for image recording materials, image recording materials using microcapsules, magnetic recording materials, micromechanical parts, plating masks, etch masks, glass fibre cable coatings, microelectronic circuits; in particular to the use of compounds of the formula I, II or III; or polymers comprising at least one repeating unit derived from the compound of the formula I, II and/or III and optionally repeating units derived from ethylenically unsaturated compounds selected from the group of formula V, as photosensitive acid donors in the preparation of surface coatings, printing inks, printing plates, dental compositions, colour filters, resists or image-recording materials, or image-recording materials for recording holographic images; as well as to a process for the preparation for the preparation of pigmented and non-pigmented surface coatings, adhesives, laminating adhesives, structural adhesives, pressure-sensitive adhesives, printing inks, printing plates, relief printing plates, planographic printing plates, intaglio printing plates, processless printing plates, screen printing stencils, dental compositions, colour filters, spacers, electroluminescence displays and liquid crystal displays (LCD), waveguides, optical switches, color proofing systems, resists, photoresists for electronics, electroplating resists, etch resists both for liquid and dry films, solder resist, photoresist materials for a UV and visible laser direct imaging system, photoresist materials for forming dielectric layers in a sequential build-up layer of a printed circuit board, image-recording materials, image-recording materials for recording holographic images, optical information storage or holographic data storage, decolorizing materials, decolorizing materials for image recording materials, image recording materials using microcapsules, magnetic recording materials, micromechanical parts, plating masks, etch masks, glass fibre cable coatings, microelectronic circuits; in particular to a process for the preparation of surface coatings, printing inks, printing plates, dental compositions, colour filters, resists, or image-recording materials, or image-recording materials for recording holographic images.

Subject of the invention is also the use of compounds of formulae I, II or III, or the polymers comprising repeating units derived from a compound of the formula I, II and/or III as photosensitive acid donors in the preparation of colour filters or chemically amplified resist materials; as well as to a process for the preparation of colour filters or chemically amplified resist materials.

The invention further pertains to a color filter prepared by providing red, green and blue picture elements and a black matrix, all comprising a photosensitive resin and a pigment and/or dye on a transparent substrate and providing a transparent electrode either on the surface of the substrate or on the surface of the color filter layer, wherein said photosensitive resin comprises compounds of formula I, II or III, or the polymers comprising repeating units derived from a compound of the formula I, II and/or III as photosensitive acid donors.

The person skilled in the art is aware of suitable pigments or dyes to provide the color elements, as well as the black matrix and corresponding suitable resins as shown in, for examples, JP-A-9-203806, JP-A-10-282650, JP-A-10-333334, JP-A-11-194494, JP-A-10-203037, JP-A-2003-5371.

As already mentioned above, in photocrosslinkable compositions, sulfonate derivatives act as latent curing catalysts: when irradiated with light they release acid which catalyses the crosslinking reaction. In addition, the acid released by the radiation can, for example, catalyse the removal of suitable acid-sensitive protecting groups from a polymer structure, or the cleavage of polymers containing acid-sensitive groups in the polymer backbone. Other applications are, for example, colour-change systems based on a change in the pH or in the solubility of, for example, a pigment protected by acid-sensitive protecting groups.

Sulfonate derivatives according to the present invention can also be used to produce so-called “print-out” images when the compound is used together with a colourant that changes colour when the pH changes, as described e.g. in JP Hei 4 328552-A or in U.S. Pat. No. 5,237,059. Such color-change systems can be used according to EP 199672 also to monitor goods that are sensitive to heat or radiation.

In addition to a colour change, it is possible during the acid-catalysed deprotection of soluble pigment molecules (as described e.g. in EP 648770, EP 648817 and EP 742255) for the pigment crystals to be precipitated; this can be used in the production of colour filters as described e.g. in EP 654711 or print out images and indicator applications, when the colour of the latent pigment precursor differs from that of the precipitated pigment crystal.

Compositions using pH sensitive dyes or latent pigments in combination with sulfonate derivatives can be used as indicators for electromagnetic radiation, such as gamma radiation, electron beams, UV- or visible light, or simple throw away dosimeters. Especially for light, that is invisible to the human eye, like UV- or IR-light, such dosimeters are of interest.

Finally, sulfonate derivatives that are sparingly soluble in an aqueous-alkaline developer can be rendered soluble in the developer by means of light-induced conversion into the free acid, with the result that they can be used as solubility enhancers in combination with suitable film-forming resins.

Resins which can be crosslinked by acid catalysis and accordingly by the photolatent acids of formula I, II or III, or the polymers comprising repeating units derived from a compound of the formula I, II and/or III according to the invention, are, for example, mixtures of polyfunctional alcohols or hydroxy-group-containing acrylic and polyester resins, or partially hydrolysed polyvinylacetals or polyvinyl alcohols with polyfunctional acetal derivatives. Under certain conditions, for example the acid-catalysed self-condensation of acetal-functionalised resins is also possible.

Suitable acid-curable resins in general are all resins whose curing can be accelerated by acid catalysts, such as aminoplasts or phenolic resole resins. These resins are for example melamine, urea, epoxy, phenolic, acrylic, polyester and alkyd resins, but especially mixtures of acrylic, polyester or alkyd resins with a melamine resin. Also included are modified surface-coating resins, such as acrylic-modified polyester and alkyd resins. Examples of individual types of resins that are covered by the expression acrylic, polyester and alkyd resins are described, for example, in Wagner, Sarx, Lackkunstharze (Munich, 1971), pp. 86-123 and pp. 229-238, or in Ullmann, Encyclopädie der techn. Chemie, 4th Ed., Vol. 15 (1978), pp. 613-628, or Ullmann's Encyclopedia of Industrial Chemistry, Verlag Chemie, 1991, Vol. 18, p. 360 ff., Vol. A19, p. 371 ff.

In coating applications the surface coating preferably comprises an amino resin. Examples thereof are etherified or non-etherified melamine, urea, guanidine or biuret resins. Acid catalysis is especially important in the curing of surface coatings comprising etherified amino resins, such as methylated or butylated melamine resins (N-methoxymethyl- or N-butoxymethyl-melamine) or methylated/butylated glycolurils. Examples of other resin compositions are mixtures of polyfunctional alcohols or hydroxy-group-containing acrylic and polyester resins, or partially hydrolysed polyvinyl acetate or polyvinyl alcohol with polyfunctional dihydropropanyl derivatives, such as derivatives of 3,4-dihydro-2H-pyran-2-carboxylic acid. Polysiloxanes can also be crosslinked using acid catalysis. These siloxane group-containing resins can, for example, either undergo self-condensation by means of acid-catalysed hydrolysis or be crosslinked with a second component of the resin, such as a polyfunctional alcohol, a hydroxy-group-containing acrylic or polyester resin, a partially hydrolysed polyvinyl acetal or a polyvinyl alcohol. This type of polycondensation of polysiloxanes is described, for example, in J. J. Lebrun, H. Pode, Comprehensive Polymer Science, Vol. 5, p. 593, Pergamon Press, Oxford, 1989. Other cationically polymerisable materials that are suitable for the preparation of surface coatings are ethylenically unsaturated compounds polymerisable by a cationic mechanism, such as vinyl ethers, for example methyl vinyl ether, isobutyl vinyl ether, trimethylolpropane trivinyl ether, ethylene glycol divinyl ether; cyclic vinyl ethers, for example 3,4-dihydro-2-formyl-2H-pyran (dimeric acrolein) or the 3,4-dihydro-2H-pyran-2-carboxylic acid ester of 2-hydroxymethyl-3,4-dihydro-2H-pyran; vinyl esters, such as vinyl acetate and vinyl stearate, mono- and di-olefins, such as a-methylstyrene, N-vinylpyrrolidone or N-vinylcarbazole.

For certain purposes, resin mixtures having monomeric or oligomeric constituents containing polymerisable unsaturated groups are used. Such surface coatings can also be cured using compounds of formula I, II or III, or the polymers comprising repeating units derived from a compound of the formula I, II and/or III. In that process, radical polymerisation initiators or photoinitiators can additionally be used. The former initiate polymerisation of the unsaturated groups during heat treatment, the latter during UV irradiation.

The invention also relates to a composition comprising

(b) as photosensitive acid donor and as compound whose solubility is increased upon the action of an acid, at least one compound of the formula I, II and/or III and/or a polymer comprising at least one repeating unit derived from a compound of the formula I, II and/or III and repeating units derived from ethylenically unsaturated compounds selected from the group of formula V.

The invention further pertains to a composition comprising

(a) a compound which cures upon the action of an acid or a compound whose solubility is increased upon the action of an acid; and
(b) as photosensitive acid donor, at least one compound of the formula I, II and/or III and/or a polymer comprising at least one repeating unit derived from a compound of the formula I, II and/or III and repeating units derived from ethylenically unsaturated compounds selected from the group of formula V.

According to the invention, the compounds of formula I, II or III, or the polymers comprising repeating units derived from a compound of the formula I, II and/or III can be used together with further photosensitive acid donor compounds (b1), further photoinitiators (d), sensitisers (e) and/or additives (c).

Suitable photosensitive acid donor compounds (b1), sensitizers (e) and additives (c) are described above.

Examples of additional photoinitiators (d) are radical photoinitiators, such as those from the class of the benzophenones, acetophenone derivatives, such as α-hydroxycycloalkylphenyl ketone, dialkoxyacetophenone, α-hydroxy- or α-amino-acetophenone, 4-aroyl-1,3-dioxolans, benzoin alkyl ethers and benzil ketals, phenylglyoxalates, dimeric phenylglyoxalates, monoacylphosphine oxides, bisacylphosphine oxides or titanocenes. Examples of especially suitable additional photoinitiators are: 1-(4-dodecylbenzoyl)-1-hydroxy-1-methyl-ethane, 1-(4-isopropylbenzoyl)-1-hydroxy-1-methyl-ethane, 1-benzoyl-1-hydroxy-1-methyl-ethane, 1-[4-(2-hydroxyethoxy)-benzoyl]-1-hydroxy-1-methyl-ethane, 1-[4-(acryloyloxyethoxy)-benzoyl]-1-hydroxy-1-methyl-ethane, diphenyl ketone, phenyl-1-hydroxy-cyclohexyl ketone, (4-morpholinobenzoyl)-1-benzyl-1-dimethylamino-propane, (4-morpholinobenzoyl)-1-(4-methylbenzyl)-1-dimethylamino-propane, 1-(3,4-dimethoxyphenyl)-2-benzyl-2-dimethylamino-butan-1-one, (4-methylthiobenzoyl)-1-methyl-1-morpholino-ethane, benzil dimethyl ketal, bis(cyclopentadienyl)-bis(2,6-difluoro-3-pyrryl-phenyl)titanium, oxo-phenyl-acetic acid 2-(2-hydroxy-ethoxy)-ethyl ester oxo-phenyl-acetic acid 1-methyl-2-[2-(2-oxo-2-phenyl-acetoxy)-propoxy]-ethyl ester, trimethylbenzoyldiphenylphosphine oxide, trimethylbenzoylphenylethoxy-phosphine oxide bis(2,6-dimethoxy-benzo-yl)-(2,4,4-trimethyl-pentyl)-phosphine oxide, bis(2,4,6-trimethylbenzoyl)-2,4-dipentyloxyphenyl-phosphine oxide or bis(2,4,6-trimethylbenzoyl)phenyl-phosphine oxide. Further suitable additional photoinitiators are to be found in U.S. Pat. No. 4,950,581, column 20, line 35 to column 21, line 35. Other examples are trihalomethyltriazine derivatives or hexaarylbisimidazolyl compounds. Further examples for additional photoinitiators are borate compounds, as for example described in U.S. Pat. No. 4,772,530, EP 775706, GB 2307474, GB 2307473 and GB 2304472. The borate compounds preferably are used in combination with electron acceptor compounds, such as, for example dye cations, or thioxanthone derivatives.

Further examples of additional photoinitiators are peroxide compounds, e.g. benzoyl peroxide (other suitable peroxides are described in U.S. Pat. No. 4,950,581, col. 19, l. 17-25) or cationic photoinitiators, such as aromatic sulfonium or iodonium salts, such as those to be found in U.S. Pat. No. 4,950,581, col. 18, l. 60 to col. 19, l. 10, or cyclopentadienyl-arene-iron(II) complex salts, for example (η⁶-isopropylbenzene)(η⁵-cyclopentadienyl)-iron(II) hexafluorophosphate.

The surface coatings may be solutions or dispersions of the surface-coating resin in an organic solvent or in water, but they may also be solventless. Of special interest are surface coatings having a low solvent content, so-called “high solids surface coatings”, and powder coating compositions. The surface coatings may be clear lacquers, as used, for example, in the automobile industry as finishing lacquers for multilayer coatings. They may also comprise pigments and/or fillers, which may be inorganic or organic compounds, and metal powders for metal effect finishes.

The surface coatings may also comprise relatively small amounts of special additives customary in surface-coating technology, for example flow improvers, thixotropic agents, leveling agents, antifoaming agents, wetting agents, adhesion promoters, light stabilisers, antioxidants, or sensitisers.

UV absorbers, such as those of the hydroxyphenyl-benzotriazole, hydroxyphenyl-benzophenone, oxalic acid amide or hydroxyphenyl-s-triazine type may be added to the compositions according to the invention as light stabilisers. Individual compounds or mixtures of those compounds can be used with or without the addition of sterically hindered amines (HALS).

Examples of such UV absorbers and light stabilisers are

1. 2-(2′-Hydroxyphenyl)-benzotriazoles, such as 2-(2′-hydroxy-5′-methylphenyl)-benzotriazole, 2-(3′,5′-di-tert-butyl-2′-hydroxyphenyl)-benzotriazole, 2-(5′-tert-butyl-2′-hydroxyphenyl)-benzotriazole, 2-(2′-hydroxy-5′-(1,1,3,3-tetramethylbutyl)phenyl)-benzotriazole, 2-(3′,5′-di-t-butyl-2′-hydroxyphenyl)-5-chloro-benzotriazole, 2-(3′-tert-butyl-2′-hydroxy-5′-methylphenyl)-5-chloro-benzotriazole, 2-(3′-sec-butyl-5′-tert-butyl-2′-hydroxyphenyl)-benzotriazole, 2-(2′-hydroxy-4′-octyloxyphenyl)-benzotriazole, 2-(3′,5′-di-tert-amyl-2′-hydroxyphenyl)-benzotriazole, 2-(3′,5′-bis-(a,a-dimethylbenzyl)-2′-hydroxyphenyl)-benzotriazole, mixture of 2-(3′-tert-butyl-2′-hydroxy-5′-(2-octyloxycarbonylethyl)phenyl)-5-chloro-benzotriazole, 2-(3′-tert-butyl-5′42-(2-ethyl-hexyloxy)-carbonylethyl′-2′-hydroxyphenyl)-5-chloro-benzotriazole, 2-(3′-tert-butyl-2′-hydroxy-5′-(2-methoxycarbonylethyl)phenyl)-5-chloro-benzotriazole, 2(3′-tert-butyl-2′-hydroxy-5′-(2-methoxycarbonylethyl)phenyl)-benzotriazole, 2-(3′-tert-butyl-2′-hydroxy-5′-(2-octyloxycarbonylethyl)phenyl)-benzotriazole, 2-(3′-tert-butyl-5′-[2-(2-ethylhexyloxy)carbonylethyl]-2′-hydroxyphenyl)-benzotriazole, 2-(3′-dodecyl-2′-hydroxy-5′-methylphenyl)-benzotriazole and 2-(3′-tert-butyl-2′-hydroxy-5′-(2-isooctyloxycarbonylethyl)phenyl-benzotriazole, 2,2′-methylene-bis[4-(1,1,3,3-tetramethylbutyl)-6-benzotriazol-2-yl-phenol]; transesterification product of 2-[3′-tert-butyl-5′-(2-methoxycarbonylethyl)-2′-hydroxy-phenyl]-benzotriazole with polyethylene glycol 300; [R—CH₂CH₂—COO(CH₂)₃]₂— wherein R=3′-tert-butyl-4′-hydroxy-5′-2H-benzotriazol-2-yl-phenyl.
2. 2-Hydroxybenzophenones, such as the 4-hydroxy, 4-methoxy, 4-octyloxy, 4-decyloxy, 4-dodecyloxy, 4-benzyloxy, 4,2′,4′-trihydroxy or 2′-hydroxy-4,4′-dimethoxy derivative.
3. Esters of unsubstituted or substituted benzoic acids, such as 4-tert-butyl-phenyl salicylate, phenyl salicylate, octylphenyl salicylate, dibenzoylresorcinol, bis(4-tert-butylbenzoyl)resorcinol, benzoylresorcinol, 3,5-di-tert-butyl-4-hydroxybenzoic acid 2,4-di-tert-butylphenyl ester, 3,5-di-tert-butyl-4-hydroxybenzoic acid hexadecyl ester, 3,5-di-tert-butyl-4-hydroxybenzoic acid octadecyl ester, 3,5-di-tert-butyl-4-hydroxybenzoic acid 2-methyl-4,6-di-tert-butylphenyl ester.
4. Acrylates, such as a-cyano-b,b-diphenylacrylic acid ethyl ester or isooctyl ester, a-carbomethoxy-cinnamic acid methyl ester, a-cyano-b-methyl-p-methoxy-cinnamic acid methyl ester or butyl ester, a-carbomethoxy-p-methoxy-cinnamic acid methyl ester, N-(b-carbomethoxy-b-cyanovinyl)-2-methyl-indoline.
5. Sterically hindered amines, such as bis(2,2,6,6-tetramethyl-piperidyl)sebacate, bis(2,2,6,-6-tetramethyl-piperidyl)succinate, bis(1,2,2,6,6-pentamethylpiperidyl)sebacate, n-butyl-3,-5-di-tert-butyl-4-hydroxybenzyl-malonic acid bis(1,2,2,6,6-pentamethylpiperidyl) ester, condensation product of 1-hydroxyethyl-2,2,6,6-tetramethyl-4-hydroxypiperidine and succinic acid, condensation product of N,N′-bis(2,2,6,6-tetramethyl-4-piperidyl)hexamethylenediamine and 4-tert-octylamino-2,6-dichloro-1,3,5-s-triazine, tris(2,2,6,6-tetramethyl-4-piperidyl)nitrilotriacetate, tetrakis(2,2,6,6-tetramethyl-4-piperidyl)-1,2,3,4-butanetetraoate, 1,1′-(1,-2-ethanediyl)-bis(3,3,5,5-tetramethyl-piperazinone), 4-benzoyl-2,2,6,6-tetramethylpiperidine, 4-stearyloxy-2,2,6,6-tetramethylpiperidine, bis(1,2,2,6,6-pentamethylpiperidyl)-2-n-butyl-2-(2-hydroxy-3,5-di-tert-butylbenzyl) malonate, 3-n-octyl-7,7,9,9-tetramethyl-1,3,8-triazaspiro[4.5]decane-2,4-dione, bis(1-octyloxy-2,2,6,6-tetramethylpiperidyl)sebacate, bis(1-octyloxy-2,2,6,6-tetramethylpiperidyl)succinate, condensation product of N,N′-bis(2,2,6,6-tetra-methyl-4-piperidyl)hexamethylenediamine and 4-morpholino-2,6-dichloro-1,3,5-triazine, condensation product of 2-chloro-4,6-di(4-n-butylamino-2,2,6,6-tetramethylpiperidyl)-1,3,5-triazine and 1,2-bis(3-aminopropylamino)ethane, condensation product of 2-chloro-4,6-di(4-n-butylamino-1,2,2,6,6-pentamethylpiperidyl)-1,3,5-triazine and 1,2-bis(3-aminopropylamino)ethane, 8-acetyl-3-dodecyl-7,7,9,9-tetramethyl-1,3,8-triazaspiro[4.5]decane-2,4-dione, 3-dodecyl-1-(2,2,6,6-tetramethyl-4-piperidyl)pyrrolidine-2,5-dione, 3-dodecyl-1-(1,2,2,6,6-pentamethyl-4-piperidyl)-pyrrolidine-2,5-dione.
6. Oxalic acid diamides, such as 4,4′-dioctyloxy-oxanilide, 2,2′-diethoxy-oxanilide, 2,2′-di-octyloxy-5,5′-di-tert-butyl-oxanilide, 2,2′-didodecyloxy-5,5′-di-tert-butyl-oxanilide, 2-ethoxy-2′-ethyl-oxanilide, N,N′-bis(3-dimethylaminopropyl)oxalamide, 2-ethoxy-5-tert-butyl-2′-ethyloxanilide and a mixture thereof with 2-ethoxy-2′-ethyl-5,4′-di-tert-butyl-oxanilide, mixtures of o- and p-methoxy- and of o- and p-ethoxy-di-substituted oxanilides.
7. 2-(2-Hydroxyphenyl)-1,3,5-triazines, such as
2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxy-phenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-[2-hydroxy-4-(2-hydroxy-3-butyloxy-propyloxy)phenyl]-4,6-bis(2,4-dimethyl-phenyl)-1,3,5-triazine, 2-[2-hydroxy-4-(2-hydroxy-3-octyloxy-propyloxy)phenyl]-4,6-bis(2,4-dimethylphenyl)-1,3,5-triazine, 2-[4-dodecyl-/tridecyl-oxy-(2-hydroxypropyl)oxy-2-hydroxy-phenyl]-4,6-bis(2,4-dimethylphenyl)-1,3,5-triazine.
8. Phosphites and phosphonites, such as triphenyl phosphite, diphenyl alkyl phosphites, phenyl dialkyl phosphites, tris(nonylphenyl) phosphite, trilauryl phosphite, trioctadecyl phosphite, distearyl-pentaerythritol diphosphite, tris(2,4-di-tert-butylphenyl) phosphite, diisodecylpentaerythritol diphosphite, bis(2,4-di-tert-butylphenyl)pentaerythritol diphosphite, bis(2,6-di-tert-butyl-4-methylphenyl)pentaerythritol diphosphite, bis-isodecyloxy-pentaerythritol diphosphite, bis(2,4-di-tert-butyl-6-methylphenyl)pentaerythritol diphosphite, bis-(2,4,6-tri-tert-butylphenyl)pentaerythritol diphosphite, tristearyl-sorbitol triphosphite, tetrakis(2,4-di-tert-butylphenyl)-4,4′-biphenylene diphosphonite, 6-isooctyloxy-2,4,8,10-tetra-tert-butyl-12H-dibenzo[d,g]-1,3,2-dioxaphosphocine, 6-fluoro-2,4,8,10-tetra-tert-butyl-12-methyl-dibenzo[d,g]-1,3,2-dioxaphosphocine, bis(2,4-di-tert-butyl-6-methylphenyl)methyl phosphite, bis(2,4-di-tert-butyl-6-methylphenyl)ethyl phosphite.

Such light stabilisers can also be added, for example, to an adjacent surface-coating layer from which they gradually diffuse into the layer of stoving lacquer to be protected. The adjacent surface-coating layer may be a primer under the stoving lacquer or a finishing lacquer over the stoving lacquer.

It is also possible to add to the resin, for example, photosensitisers which shift or increase the spectral sensitivity so that the irradiation period can be reduced and/or other light sources can be used. Examples of photosensitisers are aromatic ketones or aromatic aldehydes (as described, for example, in U.S. Pat. No. 4,017,652), 3-acyl-coumarins (as described, for example, in U.S. Pat. No. 4,366,228, EP 738928, EP 22188), keto-coumarines (as described e.g. in U.S. Pat. No. 5,534,633, EP 538997, JP 8272095-A), styryl-coumarines (as described e.g. in EP 624580), 3-(aroylmethylene)-thiazolines, thioxanthones, condensed aromatic compounds, such as perylene, aromatic amines (as described, for example, in U.S. Pat. No. 4,069,954 or WO 96/41237) or cationic and basic colourants (as described, for example, in U.S. Pat. No. 4,026,705), for example eosine, rhodanine and erythrosine colourants, as well as dyes and pigments as described for example in JP 8320551-A, EP 747771, JP 7036179-A, EP 619520, JP 6161109-A, JP 6043641, JP 6035198-A, WO 93/15440, EP 568993, JP 5005005-A, JP 5027432-A, JP 5301910-A, JP 401-4083-A, JP 429-4148-A, EP 359431, EP 103294, U.S. Pat. No. 4,282,309, EP 39025, EP 5274, EP 727713, EP 726497 or DE 2027467.

Other customary additives are—depending on the intended use—optical brighteners, fillers, pigments, colourants, wetting agents or flow improvers and adhesion promoters.

For curing thick and pigmented coatings, the addition of micro glass beads or powdered glass fibres, as described in U.S. Pat. No. 5,013,768, is suitable.

Sulfonate derivatives can also be used, for example, in hybrid systems. These systems are based on formulations that are fully cured by two different reaction mechanisms. Examples thereof are systems that comprise components that are capable of undergoing an acid-catalysed crosslinking reaction or polymerisation reaction, but that also comprise further components that crosslink by a second mechanism. Examples of the second mechanism are radical full cure, oxidative crosslinking or humidity-initiated crosslinking. The second curing mechanism may be initiated purely thermally, if necessary with a suitable catalyst, or also by means of light using a second photoinitiator. Suitable additional photoinitiators are described above.

If the composition comprises a radically crosslinkable component, the curing process, especially of compositions that are pigmented (for example with titanium dioxide), can also be assisted by the addition of a component that is radical-forming under thermal conditions, such as an azo compound, for example 2, 2′-azobis(4-methoxy-2,4-dimethylvaleronitrile), a triazene, a diazosulfide, a pentazadiene or a peroxy compound, such as, for example, a hydroperoxide or peroxycarbonate, for example tert-butyl hydroperoxide, as described, for example, in EP 245639. The addition of redox initiators, such as cobalt salts, enables the curing to be assisted by oxidative crosslinking with oxygen from the air.

The surface coating can be applied by one of the methods customary in the art, for example by spraying, painting or immersion. When suitable surface coatings are used, electrical application, for example by anodic electrophoretic deposition, is also possible. After drying, the surface coating film is irradiated. If necessary, the surface coating film is then fully cured by means of heat treatment.

The compounds of formulae I, II or III, or the polymers comprising repeating units derived from a compound of the formula I, II and/or III can also be used for curing mouldings made from composites. A composite consists of a self-supporting matrix material, for example a glass fibre fabric, impregnated with the photocuring formulation.

It is known from EP 592139 that sulfonate derivatives can be used as acid generators, which can be activated by light in compositions that are suitable for the surface treatment and cleaning of glass, aluminium and steel surfaces. The use of such compounds in organosilane systems results in compositions that have significantly better storage stability than those obtained when the free acid is used. The compounds of formula I, II or III, or the polymers comprising repeating units derived from a compound of the formula I, II and/or III are also suitable for this application.

The sulfonate derivatives of the present invention can also be used to shape polymers that undergo an acid induced transition into a state where they have the required properties using photolithography. For instance the sulfonate derivatives can be used to pattern conjugated emissive polymers as described, for example, in M. L. Renak; C. Bazan; D. Roitman; Advanced materials 1997, 9, 392. Such patterned emissive polymers can be used to manufacture microscalar patterned Light Emitting Diodes (LED) which can be used to manufacture displays and data storage media. In a similar way precursors for polyimides (e.g. polyimid precursors with acid labile protecting groups that change solubility in the developer) can be irradiated to form patterned polyimide layers which can serve as protective coatings, insulating layers and buffer layers in the production of microchips and printed circuit boards.

The formulations of the invention may also be used as conformal coatings, photoimagable insulating layers and dielectrics as they are used in sequential build up systems for printed circuit boards, stress buffer layers in the manufacturing of integrated circuits.

It is known that conjugated polymers like, e.g. polyanilines can be converted from semiconductive to conductive state by means of proton doping. The sulfonate derivatives of the present invention can also be used to imagewise irradiate compositions comprising such conjugated polymers in order to form conducting structures (exposed areas) embedded in insulating material (non exposed areas). These materials can be used as wiring and connecting parts for the production of electric and electronic devices.

Suitable radiation sources for the compositions comprising compounds of formula I, II or III, or the polymers comprising repeating units derived from a compound of the formula I, II and/or III are radiation sources that emit radiation of a wavelength of approximately from 150 to 1500, for example from 180 to 1000, or preferably from 190 to 700 nanometers as well as e-beam radiation and high-energy electromagnetic radiation such as X-rays. Both, point sources and planiform projectors (lamp carpets) are suitable. Examples are: carbon arc lamps, xenon arc lamps, medium pressure, high pressure and low pressure mercury lamps, optionally doped with metal halides (metal halide lamps), microwave-excited metal vapour lamps, excimer lamps, superactinic fluorescent tubes, fluorescent lamps, argon filament lamps, electronic flash lamps, photographic flood lights, electron beams and X-ray beams generated by means of synchrotrons or laser plasma. The distance between the radiation source and the substrate according to the invention to be irradiated can vary, for example, from 2 cm to 150 cm, according to the intended use and the type and/or strength of the radiation source. Suitable radiation sources are especially mercury vapour lamps, especially medium and high pressure mercury lamps, from the radiation of which emission lines at other wavelengths can, if desired, be filtered out. That is especially the case for relatively short wavelength radiation. It is, however, also possible to use low energy lamps (for example fluorescent tubes) that are capable of emitting in the appropriate wavelength range. An example thereof is the Philips TL03 lamp. Another type of radiation source that can be used are the light emitting diodes (LED) that emit at different wavelengths throughout the whole spectrum either as small band emitting source or as broad band (white light) source. Also suitable are laser radiation sources, for example excimer lasers, such as Kr—F lasers for irradiation at 248 nm, Ar—F lasers at 193 nm, or F₂ laser at 157 nm. Lasers in the visible range and in the infrared range can also be used. Especially suitable is radiation of the mercury i, h and g lines at wavelengths of 365, 405 and 436 nanometers. As a light source further EUV (Extreme Ultra Violet) at 13 nm is also suitable. A suitable laser-beam source is, for example, the argon-ion laser, which emits radiation at wavelengths of 454, 458, 466, 472, 478, 488 and 514 nanometers. Nd—YAG-lasers emitting light at 1064 nm and its second and third harmonic (532 nm and 355 nm respectively) can also be used. Also suitable is, for example, a helium/cadmium laser having an emission at 442 nm or lasers that emit in the UV range. With that type of irradiation, it is not absolutely essential to use a photomask in contact with the photopolymeric coating to produce a positive or negative resist; the controlled laser beam is capable of writing directly onto the coating. For that purpose the high sensitivity of the materials according to the invention is very advantageous, allowing high writing speeds at relatively low intensities. On irradiation, the sulfonate derivatives in the composition in the irradiated sections of the surface coating decompose to form the acids.

In contrast to customary UV curing with high-intensity radiation, with the compounds according to the invention activation is achieved under the action of radiation of relatively low intensity. Such radiation includes, for example, daylight (sunlight), and radiation sources equivalent to daylight. Sunlight differs in spectral composition and intensity from the light of the artificial radiation sources customarily used in UV curing. The absorption characteristics of the compounds according to the invention are as well suitable for exploiting sunlight as a natural source of radiation for curing. Daylight-equivalent artificial light sources that can be used to activate the compounds according to the invention are to be understood as being projectors of low intensity, such as certain fluorescent lamps, for example the Philips TL05 special fluorescent lamp or the Philips TL09 special fluorescent lamp. Lamps having a high daylight content and daylight itself are especially capable of curing the surface of a surface-coating layer satisfactorily in a tack-free manner. In that case expensive curing apparatus is superfluous and the compositions can be used especially for exterior finishes. Curing with daylight or daylight-equivalent light sources is an energy-saving method and prevents emissions of volatile organic components in exterior applications. In contrast to the conveyor belt method, which is suitable for flat components, daylight curing can also be used for exterior finishes on static or fixed articles and structures.

The surface coating to be cured can be exposed directly to sunlight or daylight-equivalent light sources. The curing can, however, also take place behind a transparent layer (e.g. a pane of glass or a sheet of plastics).

The examples, which follow, illustrate the invention in more detail. Parts and percentages are, as in the remainder of the description and in the claims, by weight, unless stated otherwise. Where alkyl radicals having more than three carbon atoms are referred to without any mention of specific isomers, the n-isomers are meant in each case.

### EXAMPLE 1

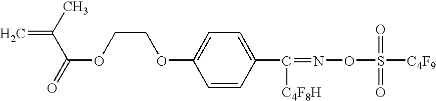

1.1:

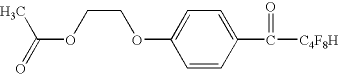

10 g (55.5 mmol) of acetic acid 2-phenoxyethyl ester are added to 60 ml of CH₂Cl₂ and cooled by ice bath. To the solution are added 25.9 g (194 mmol) of AlCl₃, followed by dropwise addition of 13.7 g (55.5 mmol) of 5H-octafluoropentanoyl chloride. The reaction mixture is stirred at room temperature overnight, poured into ice water, and extracted with CH₂Cl₂. The organic phase is washed with water, dried over MgSO₄, and concentrated. The residue is purified by column chromatography using ethyl acetate/hexane (1:9) as eluent, yielding the title compound of example 1.1 as a colorless liquid. The structure is confirmed by the ¹H-NMR spectrum (CDCl₃). δ [ppm]: 2.10 (s, 3H), 4.28 (t, 2H), 4.46 (t, 2H), 6.16 (tt, 1H), 7.01 (d, 2H), 8.08 (d, 2H).

1.2:

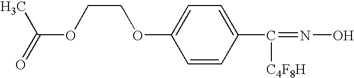

14.5 g (35.5 mmol) of the compound of example 1.2 are dissolved in 80 ml of ethanol. To the solution are added 5.92 g (85.2 mmol) of hydroxylammonium chloride and 16.9 g (213 mmol) of pyridine. The reaction mixture is refluxed overnight, and the solvent is distilled off by a rotary evaporator. The residue is poured into water, and extracted with CH₂Cl₂. The organic phase is washed with 1N HCl, water, brine, and is dried over MgSO₄. After the MgSO₄ is removed by filtration, 35 ml of 1M HCl/CH₃CO₂H is added to the solution and stirred at room temperature overnight. The reaction mixture is washed with water and brine, dried over MgSO₄, and concentrated. The product is used in the next step without further purification. The structure is confirmed by the ¹H-NMR and ¹⁹F-NMR spectrum (CDCl₃). δ [ppm]: 2.10 (s, 3H), 4.21 (t, 2H), 4.44 (t, 2H), 6.05 (tt, 1H), 6.98 (d, 2H), 7.37 (d, 2H), 8.82 (br s, 1H), −137.74 (d, 2F), −129.71 (s, 2F), −122.68 (s, 2F), −110.61 (s, 2F). The spectrum indicates that the compound is a single isomer, which is tentatively assigned as E-conformation.

1.3:

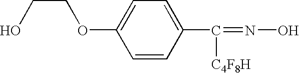

10.3 g (24.3 mmol) of the compound of example 1.2 are dissolved in 80 ml of methanol. To the solution 1.68 g (12.2 mmol) of potassium carbonate are added. The reaction mixture is stirred for 1.5 hours at r.t., poured into water, and extracted with CH₂Cl₂. The organic phase is washed with 1N HCl and water, dried over MgSO₄, and concentrated. The residue is purified by column chromatography using ethyl acetate/hexane (1:3) as eluent, yielding the title compound of example 1.3 as a colorless liquid. The structure is confirmed by the ¹H-NMR and ¹⁹F-NMR spectrum (CDCl₃). δ [ppm]: 4.00 (s, 2H), 4.12 (t, 2H), 6.05 (tt, 1H), 6.98 (d, 2H), 7.36 (d, 2H), 9.17 (s, 1H), −137.79 (d, 2F), −129.74 (s, 2F), −122.65 (s, 2F), −110.52 (s, 2F). The spectrum indicates that the compound is a single isomer, which is tentatively assigned as E-conformation.

1.4:

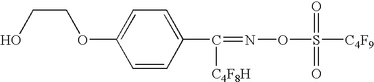

5.7 g (15.0 mmol) of the compound of example 1.3 are dissolved in 30 ml of CH₂Cl₂ and cooled in an ice bath. To the solution are added 2.41 g (22.5 mmol) of 2,6-lutidine, followed by dropwise addition of 10.5 g (18.0 mmol) of nonafluorobutanesulfonic anhydride. The reaction mixture is stirred for 3 hours at r.t., poured into ice water, and extracted with CH₂Cl₂. The organic phase is washed with 1N HCl and water, dried over MgSO₄, and concentrated. The residue is purified by column chromatography using ethyl acetate/hexane (1:3) as eluent, yielding the title compound of example 1.4 as a white solid. The structure is confirmed by the ¹H-NMR and ¹⁹F-NMR spectrum (CDCl₃). δ [ppm]: 4.01 (m, 2H), 4.16 (t, 2H), 6.06 (tt, 1H), 7.05 (d, 2H), 7.36 (d, 2H), −137.64 (d, 2F), −129.05 (s, 2F), −126.21 (s, 2F), −122.15 (s, 2F), −121.52 (s, 2F), −110.29 (s, 2F), −107.44 (s, 2F), −81.10 (s, 3F). The spectrum indicates that the compound is a single isomer, which is tentatively assigned as E-conformation.

1.5:

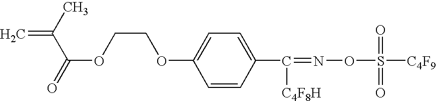

4.7 g (7.13 mmol) of the compound of example 1.4 are dissolved in 30 ml of CH₂Cl₂ and cooled in an ice bath. To the solution are added 0.89 g (8.55 mmol) of methacryloyl chloride, followed by dropwise addition of 0.94 g (9.27 mmol) of triethylamine. The reaction mixture is stirred for 1 hour at 0° C., poured into ice water, and extracted with CH₂Cl₂. The organic phase is washed with 1N HCl and water, dried over MgSO₄, and concentrated. The residue is purified by column chromatography using ethyl acetate/hexane (1:9) as eluent, yielding the title compound of example 1.5 as a colorless liquid. The structure is confirmed by the ¹H-NMR and ¹⁹F-NMR spectrum (CDCl₃). δ [ppm]: 1.95 (s, 3H), 4.30 (t, 2H), 4.53 (t, 2H), 5.60 (s, 1H), 6.06 (tt, 1H), 6.13 (s, 1H), 7.04 (d, 2H), 7.36 (d, 2H), −137.63 (d, 2F), −129.08 (s, 2F), −126.23 (s, 2F), −122.17 (s, 2F), −121.54 (s, 2F), −110.34 (s, 2F), -107.45 (s, 2F), −81.14 (s, 3F). The spectrum indicates that the compound is a single isomer, which is tentatively assigned as E-conformation.

### EXAMPLES 2-11

The compounds of examples 2 to 11 are obtained according to the method described in example 1, using the corresponding educts. The structures and physical data of products are listed in table 1.

TABLE 1

| | | Purification, |
|---|---|---|
| Ex. | Structure | Physical properties |
| 2 | 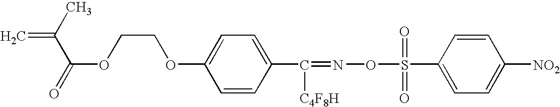 | Chromatography (ethyl acetate:hexane = 1:3) ¹H-NMR and ¹⁹F-NMR (CDCl₃) δ [ppm]: 1.95 (s, 3H), 4.28 (t, 2H), 4.53 (t, 2H), 5.60 (s, 1H), 5.99 (tt, 1H), 6.14 (s, 1H), 7.00 (d, 2H), 7.30 (d, 2H), 8.19 (d, 2H), 8.43 (d, 2H), −137.55 (d, 2F), −129.00 (s, 2F), −122.18 (s, 2F), −110.04 (s, 2F), tentatively assigned as E-configuration White solid, mp: 90-91° C. |
| 3 | 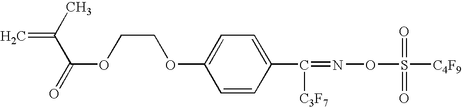 | Chromatography (ethyl acetate:hexane = 1:20) ¹H-NMR and ¹⁹F-NMR (CDCl₃). δ [ppm]: δ [ppm]: 1.95 (s, 3H), 4.29 (t, 2H), 4.53 (t, 2H), 5.60 (s, 1H), 6.14 (s, 1H), 7.06 (d, 2H), 7.36 (d, 2H), −126.26 (s, 2F), −124.71 (s, 2F), −121.52 (s, 2F), −110.74 (s, 2F), −107.37 (s, 2F), −81.13 (s, 3F), −80.48 (s, 3F), tentatively assigned as E-configuration Colorless liquid |
| 4 | 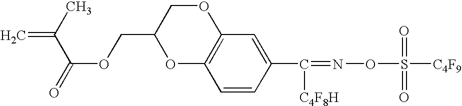 | Chromatography (ethyl acetate:hexane = 1:9) ¹H-NMR and ¹⁹F-NMR (CDCl₃). δ [ppm]: δ [ppm]: 1.95 (s, 3H), 4.10-4.19 (m, 1H), 4.37-4.57 (m, 4H), 5.63 (s, 1H), 6.06 (tt, 1H), 6.14 (s, 1H), 6.88-7.06 (m, 3H), −137.48 (d, 2F), −128.98 (s, 2F), −126.12 (s, 2F), −122.03 (s, 2F), −121.42 (s, 2F), −110.08 (s, 2F), −107.27 (s, 2F), −81.12 (s, 3F), tentatively assigned as E-configuration Colorless liquid |
| 5 | 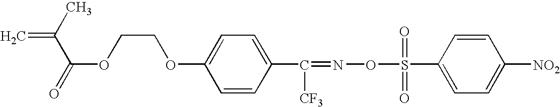 | Recrystallization from 2-propanol ¹H-NMR and ¹⁹F-NMR (CDCl₃). δ [ppm]: 1.95 (s, 3H), 4.29 (t, 2H), 4.53 (t, 2H), 5.61 (s, 1H), 6.14 (s, 1H), 7.02 (d, 2H), 7.45 (d, 2H), 8.22 (d, 2H), 8.44 (d, 2H), −66.42 (s, 3F), tentatively assigned as E-configuration Beige solid, mp: 106-109° C. |
| 6 | 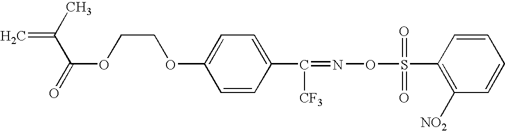 | Chromatography (ethyl acetate:hexane = 1:3) ¹H-NMR (CDCl₃) δ [ppm]: 1.96 (s, 3H), 4.30 (t, 2H), 4.53 (t, 2H), 5.61 (s, 1H), 6.14 (s, 1H), 7.04 (d, 2H), 7.60 (d, 2H), 7.81-7.91 (m, 3H), 8.28 (d, 1H), tentatively assigned as E-configuration White solid. mp: 76° C. |
| 7 | 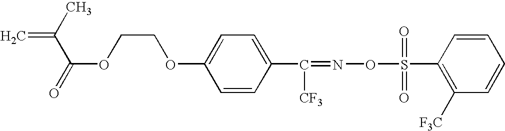 | Chromatography (ethyl acetate:hexane = 1:3) ¹H-NMR (CDCl₃) δ [ppm]: 1.95 (s, 3H), 4.30 (t, 2H), 4.53 (t, 2H), 5.61 (s, 1H), 6.14 (s, 1H), 7.02 (d, 2H), 7.48 (d, 2H), 7.80-7.90 (m, 2H), 7.96 (d, 1H), 8.38 (d, 1H), tentatively assigned as E-configuration White solid. mp: 41-47° C. |
| 8 | 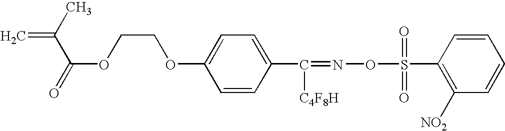 | Chromatography (ethyl acetate:hexane = 1:3) ¹H-NMR (CDCl₃) δ [ppm]: 1.94 (s, 3H), 4.28 (t, 2H), 4.51 (t, 2H), 5.29 (s, 1H), 5.93 (tt, 1H), 6.12 (s, 1H), 7.02 (d, 2H), 7.43 (d, 2H), 7.77-7.89 (m, 3H), 8.22 (d, 1H), tentatively assigned as E-configuration Orange oil |
| 9 | 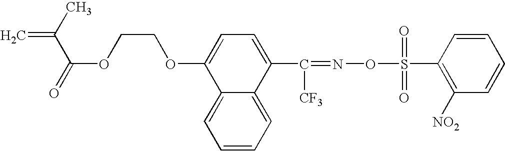 | Chromatography (ethyl acetate:hexane = 1:1) ¹H-NMR and ¹⁹F-NMR (CDCl₃). δ [ppm]: 1.95 (s, 3H), 4.38 (t, 2H), 4.63 (t, 2H), 5.58 (s, 1H), 6.14 (s, 1H), 6.85 (d, 1H), 7.36 (d, 2H), 7.42-7.45 (m, 1H), 7.51-7.57 (m, 2H), 7.74-7.83 (m, 3H), 8.21 (d, 1H). 8.31-8.35 (m, 1H), −68.45 (s, 3F), tentatively assigned as E-configuration Yellow oil |
| 10 | 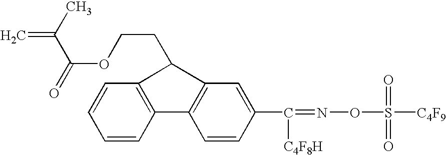 | Chromatography (ethyl acetate:hexane = 1:3) ¹H-NMR and ¹⁹F-NMR (CDCl₃). δ [ppm]: 1.82 (s, 3H), 2.35-2.53 (m, 2H), 4.02-4.16 (m, 2H), 4.19 (t, 1H), 5.47 (s, 1H), 5.87 (s, 1H), 6.09 (tt, 1H), 7.38-7.46 (m, 3H), 7.53 (s, 1H), 7.56-7.60 (m, 1H), 7.80-7.85 (m, 1H), 7.90 (d, 1H), −137.64 (d, 2F), −128.99 (s, 2F), −126.20 (s, 2F), −121.97 (s, 2F), −121.49 (s, 2F), −110.05 (s, 2F), −107.42 (s, 2F), −81.08 (s, 3F), tentatively assigned as E-configuration Pale yellow oil |
| 11 | 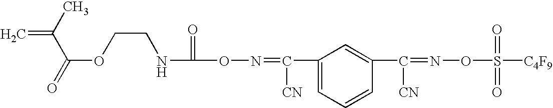 | Chromatography (ethyl acetate:hexane = 1:3) ¹H-NMR (CDCl₃) δ [ppm]: 1.96 (s, 3H), 3.66- 3.73 (m, 2H), 4.38 (t, 2H), 5.63 (s, 1H), 6.04 (t, 1H), 6.17 (s, 1H), 7.75 (t, 1H), 8.14 (d, 2H), 8.28 (d, 1H), 8.41 (s, 1H) Pale yellow solid, mp: 75-81° C. |

### EXAMPLE 12

### EAMA/BLMA/HMA/Example 1=40:40:20:2

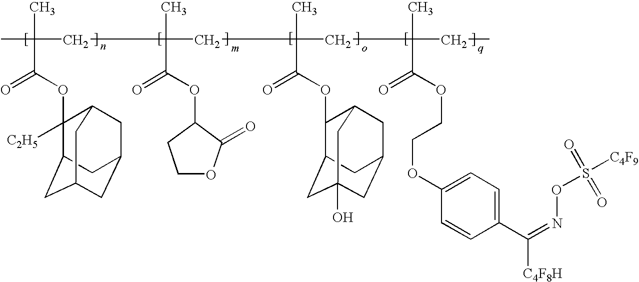

14.9 g (60 mmol) of 2-ethyl-2-adamantyl methacrylate (EAMA), 10.2 g (60 mmol) of gamma-butyrolactone methacrylate (BLMA), 7.1 g (30 mmol) of 3-hydroxy-1-adamantyl methacrylate (HMA), 2.19 g (3 mmol) of Example 1 and 3.5 g (15 mmol) of a polymerization initiator, V-601 produced by Wako Pure Chemical Industries, Ltd., are dissolved in 300 ml of THF, and then polymerization is carried out for 3 hours under an atmosphere of nitrogen at reflux conditions. The polymerization solution is poured into hexane, yielding a white precipitation. The white powder is filtered, dissolved in THF again, re-precipitated in CH₃OH/H₂O solution, filtered and dried under vacuum. By GPC measurement using polystyrene standard, the weight average molecular weight (Mw) and the number average molecular weight (Mn) of the polymer obtained are 8700 and 4800, respectively.

### EXAMPLE 13

### EAMA/BLMA/HMA/Example 1=40:40:20:5

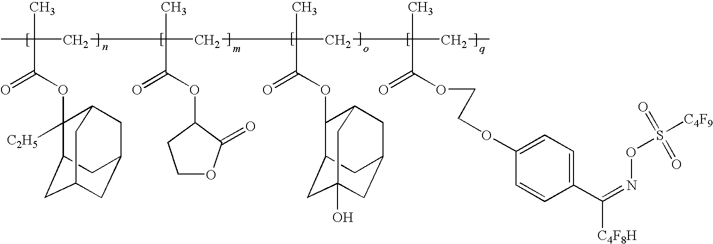

14.9 g (60 mmol) of EAMA, 10.2 g (60 mmol) of BLMA, 7.1 g (30 mmol) of HMA, 5.5 g (7.5 mmol) of Example 1 and 3.5 g (15 mmol) of a polymerization initiator, V-601 produced by Wako Pure Chemical Industries, Ltd., are dissolved in 300 ml of THF, and then polymerization is carried out for 3 hours under an atmosphere of nitrogen at reflux conditions. The polymerization solution is poured into hexane, yielding a white precipitation. The white powder is filtered, dissolved in THF again, re-precipitated in CH₃OH/H₂O solution, filtered and dried under vacuum. By GPC measurement using polystyrene standard, the weight average molecular weight (Mw) and the number average molecular weight (Mn) of the polymer obtained are 9800 and 5300, respectively.

### EXAMPLE 14

### EAMA/BLMA/HMA/Example 1=40:20:40:2

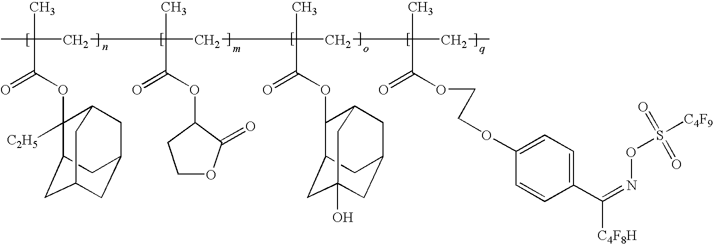

24.8 g (100 mmol) of EAMA, 9.6 g (50 mmol) of BLMA, 23.6 g (100 mmol) of HMA, 3.66 g (5 mmol) of Example 1 and 5.8 g (25 mmol) of a polymerization initiator, V-601 produced by Wako Pure Chemical Industries, Ltd., are dissolved in 300 ml of THF, and then polymerization is carried out for 3 hours under an atmosphere of nitrogen at reflux conditions. The polymerization solution is poured into hexane, yielding a white precipitation. The white powder is filtered, dissolved in THF again, re-precipitated in CH₃OH/H₂O solution, filtered and dried under vacuum. By GPC measurement using polystyrene standard, the weight average molecular weight (Mw) and the number average molecular weight (Mn) of the polymer obtained are 9900 and 5600, respectively.

### EXAMPLE 15

### EAMA/BLMA/HMA/Example 1=40:20:40:5

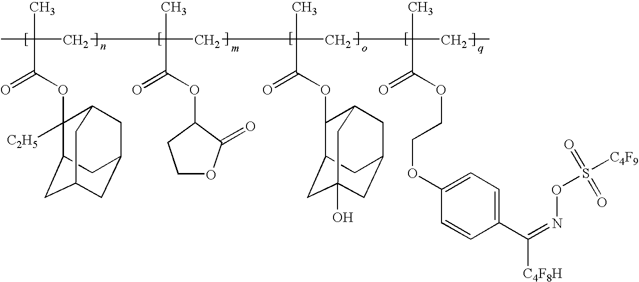

24.8 g (100 mmol) of EAMA, 9.6 g (50 mmol) of BLMA, 23.6 g (100 mmol) of HMA, 9.1 g (12.5 mmol) of Example 1 and 5.8 g (25 mmol) of a polymerization initiator, V-601 produced by Wako Pure Chemical Industries, Ltd., are dissolved in 300 ml of THF, and then polymerization is carried out for 3 hours under an atmosphere of nitrogen at reflux conditions. The polymerization solution is poured into hexane, yielding a white precipitation. The white powder is filtered, dissolved in THF again, re-precipitated in CH₃OH/H₂O solution, filtered and dried under vacuum. By GPC measurement using polystyrene standard, the weight average molecular weight (Mw) and the number average molecular weight (Mn) of the polymer obtained are 11000 and 6200, respectively.

### EXAMPLE 16

### EAMA/BLMA/HMA/Example 2=40:20:40:5

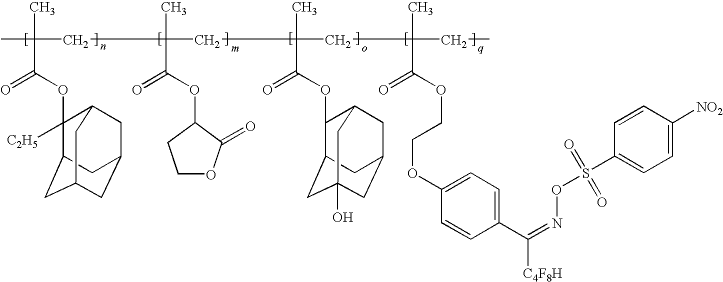

2.48 g (10 mmol) of EAMA, 0.96 g (5 mmol) of BLMA, 2.36 g (10 mmol) of HMA, 0.79 g (1.25 mmol) of Example 2 and 0.58 g (2.5 mmol) of a polymerization initiator, V-601 produced by Wako Pure Chemical Industries, Ltd., are dissolved in 30 ml of THF, and then polymerization is carried out for 3 hours under an atmosphere of nitrogen at reflux conditions. The polymerization solution is poured into hexane, yielding a white precipitation. The white powder is filtered, dissolved in THF again, re-precipitated in CH₃OH/H₂O solution, filtered and dried under vacuum. By GPC measurement using polystyrene standard, the weight average molecular weight (Mw) and the number average molecular weight (Mn) of the polymer obtained are 12000 and 7300, respectively.

### EXAMPLE 17

### EAMA/BLMA/HMA/Example 2=40:40:20:5

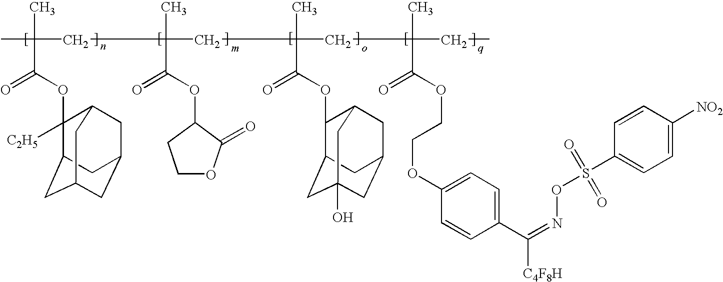

3.68 g (14.8 mmol) of EAMA, 2.51 g (14.8 mmol) of BLMA, 1.45 g (7.4 mmol) of HMA, 1.17 g (1.85 mmol) of Example 2 and 0.86 g (3.7 mmol) of a polymerization initiator, V-601 produced by Wako Pure Chemical Industries, Ltd., are dissolved in 45 ml of THF, and then polymerization is carried out for 3 hours under an atmosphere of nitrogen at reflux conditions. The polymerization solution is poured into hexane, yielding a white precipitation. The white powder is filtered, dissolved in THF again, re-precipitated in CH₃OH/H₂O solution, filtered and dried under vacuum. By GPC measurement using polystyrene standard, the weight average molecular weight (Mw) and the number average molecular weight (Mn) of the polymer obtained are 9700 and 5200, respectively.

### EXAMPLES 18-31

The compounds of examples 18 to 31 are obtained according to the method described in example 12, using the corresponding educts. The structures and molecular weight of products are listed in table 2.

TABLE 2

| | | Monomer feeding ratio, |
|---|---|---|
| | | weight average molecular |
| | | weight (Mw), number |
| | | average molecular weight |
| Ex. | Structure | (Mn) |
| 18 | 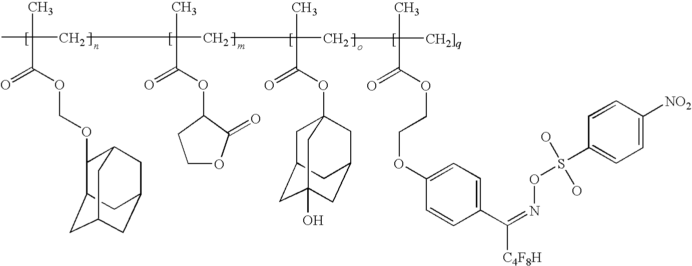 | AOMM/BLMA/HMA/ Example 2 = 40:40:20:1.7 Mw = 8800 Mn = 5500 |
| 19 | 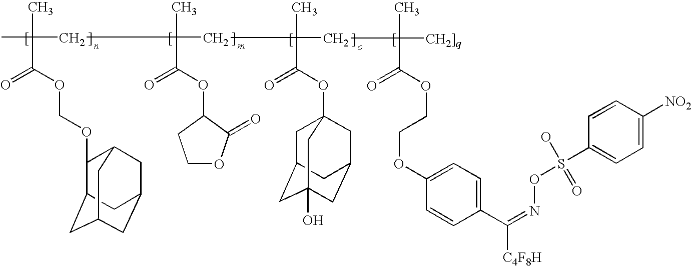 | AOMM/BLMA/HMA/ Example 2 = 40:40:20:3.4 Mw = 10400 Mn = 6500 |
| 20 | 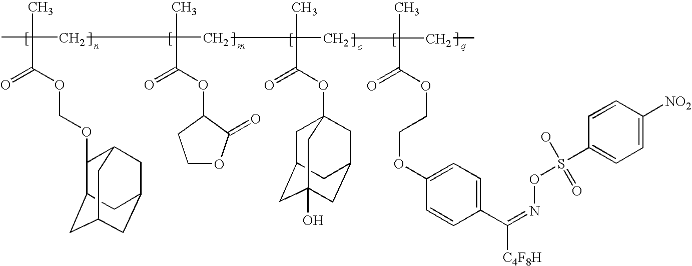 | AOMM/BLMA/HMA/ Example 2 = 40:40:20:6.8 Mw = 7900 Mn = 5500 |
| 21 | 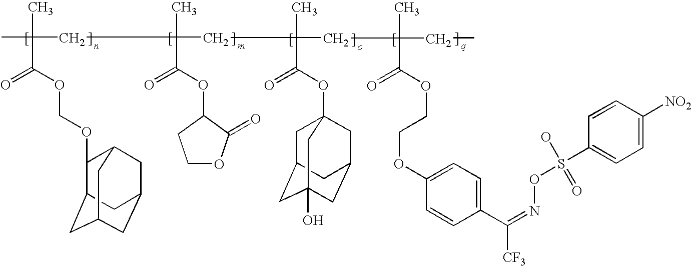 | AOMM/BLMA/HMA/ Example 5 = 40:40:20:4.3 Mw = 14400 Mn = 8400 |
| 22 | 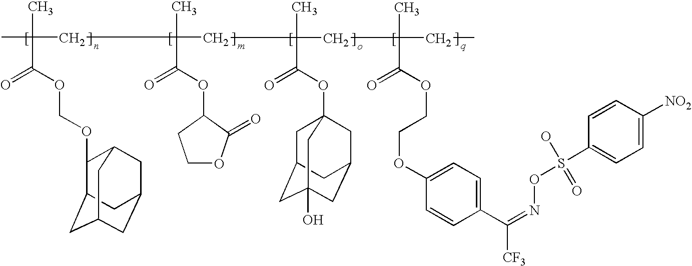 | AOMM/BLMA/HMA/ Example 5 = 40:40:20:8.6 Mw = 15000 Mn = 8800 |
| 23 | 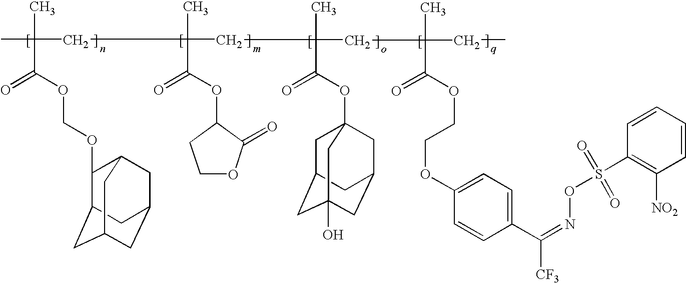 | AOMM/BLMA/HMA/ Example 6 = 40:40:20:4.3 Mw = 7100 Mn = 5000 |
| 24 | 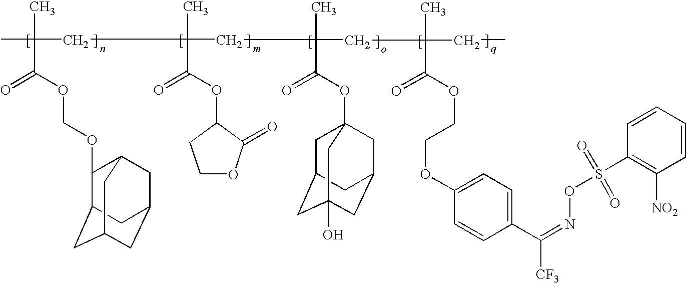 | AOMM/BLMA/HMA/ Example 6 = 40:40:20:8.6 Mw = 7200 Mn = 4500 |
| 25 | 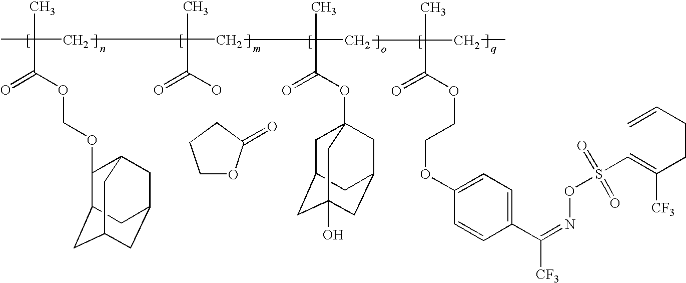 | AOMM/BLMA/HMA/ Example 7 = 40:40:20:4.3 Mw = 11300 Mn = 6900 |
| 26 | 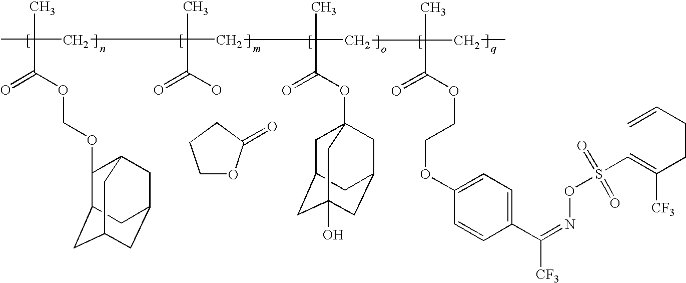 | AOMM/BLMA/HMA/ Example 7 = 40:40:20:8.6 Mw = 10700 Mn = 6600 |
| 27 | 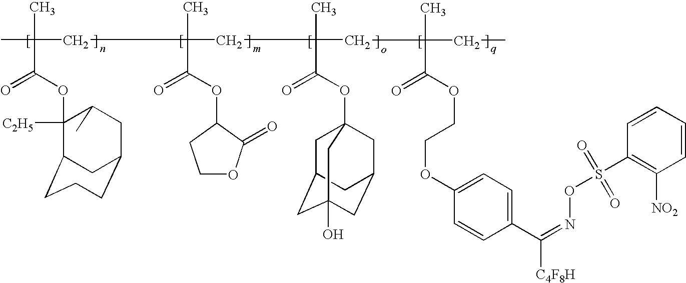 | EAMA/BLMA/HMA/ Example 8 = 40:40:20:6.8 Mw = 10000 Mn = 6900 |
| 28 | 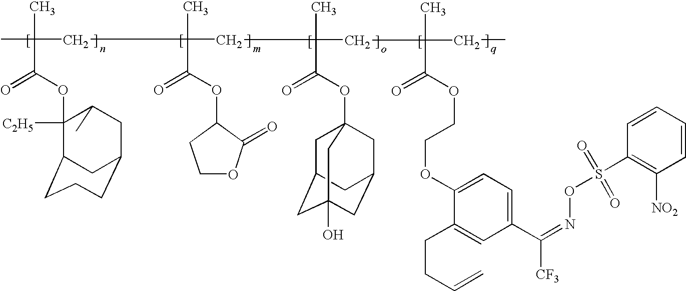 | EAMA/BLMA/HMA/ Example 9 = 40:40:20:7.8 Mw = 11000 Mn = 7600 |
| 29 | 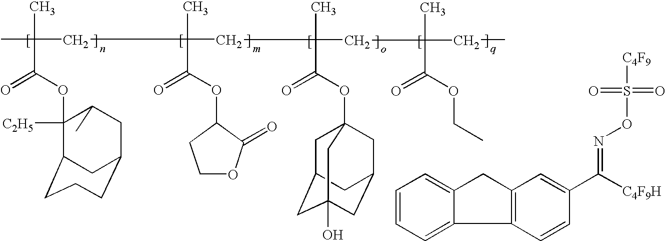 | EAMA/BLMA/HMA/ Example 10 = 40:40:20:2.7 Mw = 8100 Mn = 4100 |
| 30 | 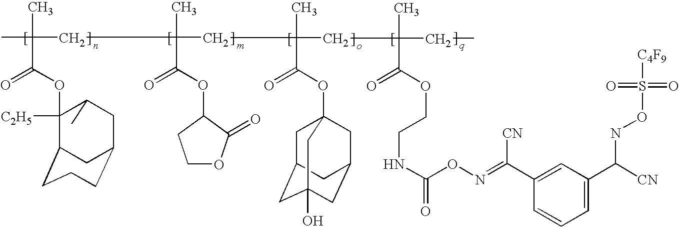 | EAMA/BLMA/HMA/ Example 11 = 40:40:20:2 Mw = 3900 Mn = 2600 |
| 31 | 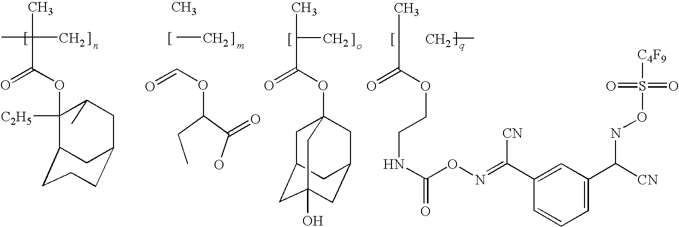 | EAMA/BLMA/HMA/ Example 11 = 40:40:20:5 Mw = 3000 Mn = 2000 |

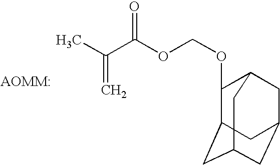

### EXAMPLE 32

Positive type photoresist compositions are prepared by mixing and dissolving the components shown in Table 3. Each of the positive tone photoresist composition is evaluated in lithography properties by forming the resist patterns with the procedure disclosed below.

TABLE 3

Components:

| | Photoacid | | |
|---|---|---|---|
| | generator | Additive | Solvent |
| Formulation 1 | Example 12 | (c)-1 | (s)-1 |
| | [100] | [0.5] | [2000] |
| Formulation 2 | Example 17 | (c)-1 | (s)-1 |
| | [100] | [0.5] | [2000] |
| Formulation 3 | Example 19 | (c)-1 | (s)-2 |
| | [100] | [0.5] | [2000] |
| Formulation 4 | Example 20 | (c)-1 | (s)-2 |
| | [100] | [1.0] | [2000] |
| Formulation 5 | Example 20 | (c)-1 | (s)-2 |
| | [100] | [0.5] | [2000] |
| Formulation 6 | Example 27 | (c)-1 | (s)-2 |
| | [100] | [1.0] | [2000] |
| Formulation 7 | Example 27 | (c)-1 | (s)-2 |
| | [100] | [0.5] | [2000] |
| Formulation 8 | Example 29 | (c)-1 | (s)-2 |
| | [100] | [0.5] | [2000] |
| Formulation 9 | Example 29 | (c)-1 | (s)-2 |
| | [100] | [0.2] | [2000] |

(c)-1: tri(n-pentyl)amine
(s)-1: mixture of solvents of PGMEA and PGME (8:2 by weight)
(s)-2: mixture of solvents of PGMEA and PGME (6:4 by weight)
numbers in [ ] indicate amount of the components as parts by weight

On an eight inches semiconductor silicon wafer is coated a composition comprising organic compounds for anti-reflection coating, ARC₂₉A™ (by Brewer Science) by using a spinner, followed by drying and baking treatment for 60 seconds on a hot plate at 205° C. to form an antireflective coating having a thickness of 77 nm. On the coating, the positive tone resist compositions are coated by using a spinner followed by drying and post applied baking treatment for 60 seconds on a hot plate at the PAB temperatures listed in Table 3 to form a photoresist layer having a thickness of 150 nm. The photoresist layer is pattern-wise exposed to ArF excimer laser beams of 193 nm wavelength on an ArF exposure machine NSR-S302(manufactured by Nikon Co.; NA (Numerical Aperture)=0.60, ⅔ Annular illumination) through a mask pattern (6% halftone reticule).

Then the layer is post exposure baked for 60 seconds on a hot plate at the PEB temperatures listed in Table 4, developed at 23° C. for 30 seconds with a 2.38% by weight aqueous solution of tetramethylammonium hydroxide followed by rinse with water for 30 seconds and drying to form the resist patters.

### Sensitivity

To form a resist pattern of 1:1 line and space (L/S pattern), the optimized exposure dose for the L/S pattern (line width: 120 nm, pitch: 240 nm) is determined (photosensitivity: Eₒₚ, mJ/cm²).

### Resolution

The finest feature size of the photoresist is determined by changing the size of the mask pattern in the previous Eₒₚ determination.

TABLE 4

| | E_{OP} | PAB temperature/ | Resolution |
|---|---|---|---|
| | (mJ/cm²) | PEB temperature (° C.) | (nm) |
| Formulation 1 | 20 | 110/110 | 110 |
| Formulation 2 | 50 | 110/110 | 120 |
| Formulation 3 | 120 | 110/110 | 110 |
| Formulation 4 | 100 | 110/110 | 110 |
| Formulation 5 | 54 | 110/110 | 110 |
| Formulation 6 | 49 | 110/110 | 120 |
| Formulation 7 | 30 | 110/110 | 120 |
| Formulation 8 | 13 | 110/110 | 120 |
| Formulation 9 | 6 | 110/110 | 120 |

Obviously from the results mentioned above, it is confirmed that a hyperfine resist pattern can be formed with the positive tone photoresist composition described in Example 32.

### Measurement of Leachate

Positive type photoresist compositions are prepared by mixing and dissolving the components shown in Table 5.

TABLE 5

Components:

| | Photoacid generator | Polymer | Solvent |
|---|---|---|---|
| Formulation 10 | Example 27 | | (s)-2 |
| | [100] | | [2000] |
| Formulation 11 | Example 28 | | (s)-2 |
| | [100] | | [2000] |
| Formulation 12 | Example 29 | | (s)-2 |
| | [100] | | [2000] |
| Comparative | (b)-1 | (a)-1 | (s)-2 |
| formulation 1 | [10] | [100] | [2000] |

(a)-1: copolymer of 2-methaclyloyloxy-2-ethyladamantane/alpha-meth-acryloyloxy-gamma-butyrolactone/1-methaclyloyloxy-3-hydroxylada-mantane (4:4:2 molar ratio, Mw = 10000, Mn = 5600)
(b)-1: 4-methylphenyldiphenylsulfonium nonafluoro-n-butanesulfonate
(s)-2: mixture of solvents of PGMEA and PGME (6:4 by weight)

The resist films are prepared according to the method described above with positive type photoresist Formulation 10˜12 and Comparative formulation 1. Next a droplet of pure water (50 μL) is moved from the center of wafer in a circular motion at constant linear speed by VRC₃₁₀S (available from S. E. S. Co. Ltd.) at room temperature. The total contact area of resist film on which the droplet contacted is 221.56 cm².

Then the droplet is corrected and analyzed by Agilent-HP1100 LC-MSD (available from Agilent Technologies Inc.) to determine the amount of leachate (mol/cm²) from the resist film before exposed.

The results are summarized in Table 6.

TABLE 6

| | Amount of leachate (× 10⁻¹² mol/cm²): |
|---|---|
| | (Before exposure) |
| Formulation 10 | 0 |
| Formulation 11 | 0 |
| Formulation 12 | 0.18 |
| Comparative | 22.54 |
| formulation 1 | |

The amount of leachate from Formulation 10˜12 is much less than that from Comparative formulation 1. Therefore it is found that positive type photoresist compositions of this invention are suitable for immersion exposure because they can suppress the leaching in immersion exposure.

1. A compound of the formula I, II or III 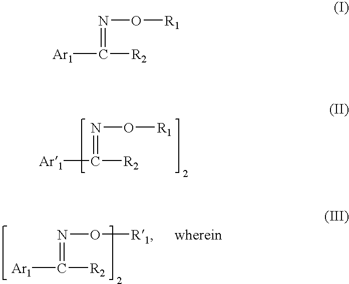
R₁ is C₁-C₁₈alkylsulfonyl, C₁-C₁₀haloalkylsulfonyl,
C₂-C₁₂alkenylsulfonyl, C₂-C₁₂alkynylsulfonyl, C₃-C₃₀cycloalkylsulfonyl, C₁-C₁₈alkylsulfonyl interrupted by one or more O or C₁-C₁₀haloalkylsulfonyl interrupted by one or more O;
wherein the groups C₁-C₁₈alkylsulfonyl, C₁-C₁₀haloalkylsulfonyl, C₂-C₁₂alkenylsulfonyl, C₂-C₁₂alkynylsulfonyl, C₃-C₃₀cycloalkylsulfonyl, interrupted C₁-C₁₈alkylsulfonyl and interrupted C₁-C₁₀haloalkylsulfonyl optionally are substituted by one or more C₃-C₃₀cycloalkyl, C₄-C₃₀cycloalkenyl, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or are substituted by NO₂, CN, Ar₂, (CO)R₇, (CO)OR′₃, (CO)NR₄R₅, O(CO)R₇, O(CO)OR′₃, O(CO)NR₄R₆, NR₆(CO)R₇, NR₆(CO)OR′₃, OR′₃, NR₄R₅, SR₆, SOR₇, SO₂R₇ and/or OSO₂R₇;
or R₁ is camphorylsulfonyl, phenyl-C₁-C₃alkylsulfonyl, phenylsulfonyl, naphthylsulfonyl, anthrylsulfonyl, phenanthrylsulfonyl or heteroarylsulfonyl,
wherein the groups camphorylsulfonyl, phenyl-C₁-C₃alkylsulfonyl, phenylsulfonyl, naphthylsulfonyl, anthrylsulfonyl, phenanthrylsulfonyl and heteroarylsulfonyl optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆ CO, SO and/or SO₂, C₃-C₃₀cycloalkyl which is interrupted by one or more O, S, NR₆ CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or are substituted by halogen, NO₂, CN, Ar₂, (CO)R₇, (CO)OR′₃, (CO)NR₄R₅, O(CO)R₇, O(CO)OR′₃, O(CO)NR₄R₆, NR₆(CO)R₇, NR₆(CO)OR′₃, OR′₃, NR₄R₆, SR₆, SOR₇, SO₂R₇ and/or OSO₂R₇;
wherein all radicals R₁ optionally additionally are substituted by a group having a —O—C-bond or a —O—Si-bond which cleaves upon the action of an acid;
R′₁, is phenylenedisulfonyl, naphthylenedisulfonyl, 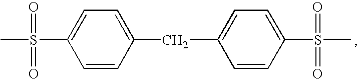
diphenylenedisulfonyl or oxydiphenylenedisulfonyl,
wherein the groups phenylenedisulfonyl, naphthylenedisulfonyl, 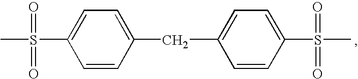
diphenylenedisulfonyl and oxydiphenylenedisulfonyl optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or are substituted by halogen, NO₂, CN, Ar₂, (CO)R₂, (CO)OR′₃, (CO)NR₄R₅, O(CO)R₇, O(CO)OR′₃, O(CO)NR₄R₅, NR₆(CO)R₂, NR₆(CO)OR′₃, OR′₃, NR₄R₅, SR₆, SOR₇, SO₂R₇ and/or OSO₂R₇;
or R′₁ is C₁-C₁₂alkylenedisulfonyl or C₁-C₁₀haloalkylenedisulfonyl;
wherein all radicals R′₁, optionally additionally are substituted by a group having a —O—C-bond or a —O—Si-bond which cleaves upon the action of an acid;
R₂ is CN, C₁-C₁₀haloalkyl or C₁-C₁₀haloalkyl which is substituted by NO₂, CN, Ar₂, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₂, SO₂R₂, OSO₂R₇, and/or a group of formula IV 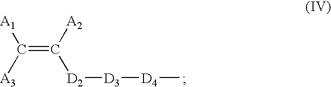
Ar₁ is phenyl, biphenylyl, fluorenyl, naphthyl, anthryl, phenanthryl or heteroaryl, wherein the groups phenyl, biphenylyl, fluorenyl, naphthyl, anthryl, phenanthryl and heteroaryl optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂; or are substituted by a group of formula IV 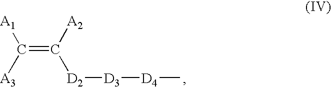
halogen, NO₂, CN, Ar₂, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇;
optionally the substituents C₁-C₁₈alkyl, C₂-C₁₂alkenyl, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇ form 5-, 6- or 7-membered rings, via the radicals C₁-C₁₈alkyl, C₂-C₁₂alkenyl, R₃, R₄, R₆, R₆ and/or R₇, with further substituents on the phenyl, biphenylyl, naphthyl, anthryl, phenanthryl, or heteroaryl ring or with one of the carbon atoms of the phenyl, biphenylyl, naphthyl, anthryl, phenanthryl, or heteroaryl ring;
wherein all radicals Ar₁ optionally additionally are substituted by a group having a —O—C-bond or a —O—Si-bond which cleaves upon the action of an acid;
Ar₁ is phenylene, biphenylene, naphthylene, 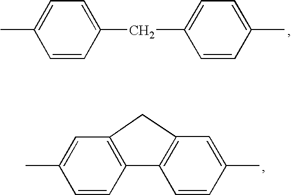
heteroarylene, oxydiphenylene or 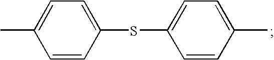
wherein the groups phenylene, biphenylene, naphthylene, 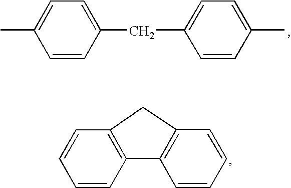
heteroarylene, oxydiphenylene and 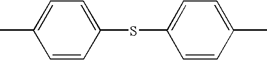
optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or are substituted by a group of formula IV 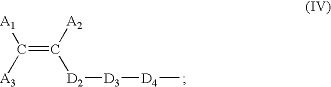
or are substituted by halogen, NO₂, CN, Ar₂, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇,
optionally the substituents C₁-C₁₈alkyl, C₂-C₁₂alkenyl, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇ form 5-, 6- or 7-membered rings, via the radicals C₁-C₁₈alkyl, C₂-C₁₂alkenyl, R₃, R₄, R₅, R₆ and/or R₇, with further substituents on the phenylene, biphenylene, naphthylene, 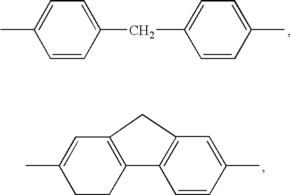
heteroarylene, 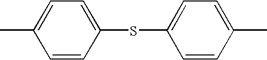
or oxydiphenylene ring or with one of the carbon atoms of the phenylene, biphenylene, naphthylene, 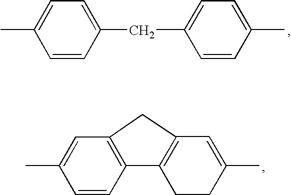
heteroarylene, 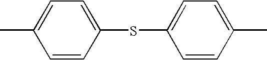
or oxydiphenylene ring;
or Ar′₁ is —Ar″₁—X₁—Y₁—X₁—Ar″₁—;
wherein all radicals Ar′₁ optionally additionally are substituted by a group having a —O—C-bond or a —O—Si-bond which cleaves upon the action of an acid,
Ar″₁ is phenylene, biphenylene, naphthylene, heteroarylene;
wherein the groups phenylene, biphenylene, naphthylene, heteroarylene optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or are substituted by halogen, NO₂, CN, Ar₂, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇,
optionally the substituents C₁-C₁₈alkyl, C₂-C₁₂alkenyl, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₇, SO₂R₇ and/or OSO₂R₇ form 5-, 6- or 7-membered rings, via the radicals C₁-C₁₈alkyl, C₂-C₁₂alkenyl, R₃, R₄, R₅, R₆ and/or R₇, with further substituents on the phenylene, biphenylene, naphthylene, heteroarylene ring or with one of the carbon atoms of the phenylene, biphenylene, naphthylene, heteroarylene ring;
wherein all radicals A″₁ optionally additionally are substituted by a group having a —O—C-bond or a —O—Si-bond which cleaves upon the action of an acid;
X₁ is a direct bond, O, S, NR₆, CO, O(CO), S(CO), NR₆(CO), SO, SO₂, or OSO₂;
or X₁ is C₁-C₁₈alkylene or phenylene wherein these radicals are unsubstituted or substituted by one or more C₁-C₁₈alkyl, C₁-C₄haloalkyl, halogen, OR₃ and/or SR₆;
Y₁ is C₁-C₁₈alkylene which optionally is substituted by OR₃, SR₆, halogen, phenyl and/or a group of formula IV 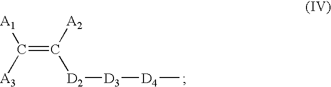
or Y₁ is C₂-C₁₈alkylene which is interrupted by one or more O, S, NR₅, CO SO and/or SO₂ and which optionally is substituted by a group of formula IV 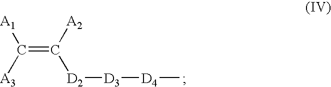
R′₃ is hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl;
or R′₃ is C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or R′₃ is phenyl, naphthyl, C₂-C₁₈alkanoyl, benzoyl, C₁-C₁₈alkylsulfonyl, phenylsulfonyl, naphthylsulfonyl, anthrylsulfonyl or phenanthrylsulfonyl;
wherein the groups phenyl, naphthyl, C₂-C₁₈alkanoyl, benzoyl, C₁-C₁₈alkylsulfonyl, phenylsulfonyl, naphthylsulfonyl, anthrylsulfonyl and phenanthrylsulfonyl optionally are substituted by one or more Ar₂, OH, C₁-C₁₈alkyl, C₁-C₁₀haloalkyl, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy and/or benzoyloxy;
R₃ is C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl; all of which optionally are substituted by a group of formula IV 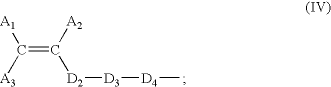
or R₃ is C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or R₃ is phenyl, naphthyl, C₂-C₁₈alkanoyl, benzoyl, C₁-C₁₈alkylsulfonyl, phenylsulfonyl, naphthylsulfonyl, anthryl sulfonyl or phenanthrylsulfonyl;
wherein the groups phenyl, naphthyl, C₂-C₁₈alkanoyl, benzoyl, C₁-C₁₈alkylsulfonyl, phenylsulfonyl, naphthylsulfonyl, anthrylsulfonyl and phenanthrylsulfonyl optionally are substituted by one or more Ar₂, OH, C₁-C₁₈alkyl, C₁-C₁₀haloalkyl, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy, benzoyloxy and/or a group of formula IV 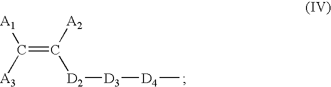
or R₃ is hydrogen;
R₄ and R₅ independently of each other are hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or R₄ and R₅ independently of each other are phenyl, naphthyl, C₂-C₁₈alkanoyl, benzoyl, C₁-C₁₈alkylsulfonyl, phenylsulfonyl, naphthylsulfonyl, anthrylsulfonyl or phenanthrylsulfonyl;
wherein the groups phenyl, naphthyl, C₂-C₁₈alkanoyl, benzoyl, C₁-C₁₈alkylsulfonyl, phenylsulfonyl, naphthylsulfonyl, anthrylsulfonyl and phenanthrylsulfonyl optionally are substituted by one or more Ar₂, OH, C₁-C₁₈alkyl, C₁-C₁₀haloalkyl, halogen, NO₂, CN, C₁-C₁₈alkoxy, phenoxy, C₁-C₁₈alkylamino, C₁-C₁₈dialkylamino, C₁-C₁₂alkylthio, C₁-C₁₈alkylsulfonyloxy, phenylsulfonyloxy, (4-methylphenyl)sulfonyloxy, C₂-C₁₈alkanoyloxy, and/or by benzoyloxy;
or R₄ and R₅, together with the nitrogen atom to which they are attached, form a 5-, 6- or 7-membered ring which optionally is interrupted by one or more O, NR₈ or CO;
R₆ is hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, C₂-C₁₈alkyl which is interrupted by one or more O, S, NR₈, CO, SO and/or SO₂, C₃-C₃₀cycloalkyl which is interrupted by one or more O, S, NR₈, CO, SO and/or SO₂, C₄-C₃₀cycloalkenyl which is interrupted by one or more O, S, NR₈, CO, SO and/or SO₂, C₂-C₁₂alkenyl which is interrupted by one or more O, S, NR₈, CO, SO and/or SO₂;
or R₆ is phenyl, naphthyl, C₂-C₁₈alkanoyl, benzoyl, C₁-C₁₈alkylsulfonyl, phenylsulfonyl, naphthylsulfonyl, anthrylsulfonyl or phenanthrylsulfonyl;
wherein the groups phenyl, naphthyl, C₂-C₁₈alkanoyl, benzoyl, C₁-C₁₈alkylsulfonyl, phenylsulfonyl, naphthylsulfonyl, anthrylsulfonyl and phenanthrylsulfonyl optionally are substituted by one or more Ar₂, OH, C₁-C₁₈alkyl, C₁-C₁₀haloalkyl, halogen, NO₂, CN, C₁-C₁₈alkoxy, phenoxy, NR₄R₆, C₁-C₁₂alkylthio, C₁-C₁₈alkylsulfonyloxy, phenylsulfonyloxy, (4-methylphenyl)sulfonyloxy, C₂-C₁₈alkanoyloxy and/or by benzoyloxy;
R₇ is hydrogen, C₃-C₃₀cycloalkyl, C₂-C₁₂alkenyl, C₄-C₃₀cycloalkenyl, phenyl-C₁-C₃-alkyl, C₂-C₁₈alkyl which is interrupted by one or more O, S, NR₆, CO, SO and/or SO₂, C₃-C₃₀cycloalkyl which is interrupted by one or more O, S, NR₆, CO, SO and/or SO₂, C₄-C₃₀cycloalkenyl which is interrupted by one or more O, S, NR₆, CO, SO and/or SO₂, C₂-C₁₂alkenyl which is interrupted by one or more O, S, NR₆, CO, SO and/or SO₂;
or R₇ is phenyl, or naphthyl both of which optionally are substituted by one or more Ar₂, OH, C₁-C₁₈alkyl, C₁-C₁₀haloalkyl, halogen, NO₂, CN, C₁-C₁₈alkoxy, phenoxy, NR₄R₆, C₁-C₁₂alkylthio, C₁-C₁₈alkylsulfonyloxy, phenylsulfonyloxy, (4-methylphenyl)sulfonyloxy, C₂-C₁₈alkanoyloxy and/or by benzoyloxy;
R₈ is C₃-C₃₀cycloalkyl, C₁-C₁₈alkyl, C₁-C₁₀haloalkyl, C₂-C₁₂alkenyl, C₄-C₃₀cycloalkenyl or phenyl-C₁-C₃alkyl;
Ar₂ is phenyl, biphenylyl or naphthyl,
wherein the groups phenyl, biphenylyl and naphthyl optionally are substituted by one or more OH, C₁-C₁₈alkyl, C₁-C₁₀haloalkyl, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy and/or by benzoyloxy;
A₁, A₂ and A₃ independently of each other are hydrogen, halogen, CN, C₁-C₁₈alkyl, C₁-C₁₈alkyl which is substituted by OR₃; or A₁, A₂ and A₃ independently of each other are C₁-C₁₀haloalkyl, (CO)R₇, (CO)OR₃, or (CO)NR₄R₅;
D₂ is a direct bond, O, (CO)O, (CO)S, (CO)NR₆, SO₂, OSO₂, Ar′₂, C₁-C₁₈alkylene;
or A₃ and D₂ together with the ethylenically unsaturated double bond to which they are attached form C₃-C₃₀cycloalkenyl which optionally is interrupted by one or more O, S, N, NR₆, CO, SO and/or SO₂;
or A₂ and D₂ together with the carbon of the ethylenically unsaturated double bond to which they are attached form C₃-C₃₀cycloalkyl which optionally is interrupted by one or more O, S, N, NR₆, CO, SO and/or SO₂;
D₃ and D₄ independently of each other are a direct bond, O, S, NR₆, CO, O(CO), (CO)O, (CO)S, (CO)NR₆, SO, SO₂, OSO₂, Ar′₂, 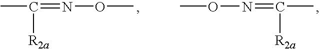
C₁-C₁₈alkylene, C₃-C₃₀cycloalkylene, C₂-C₁₂alkenylene, C₄-C₃₀cycloalkenylene, C₂-C₁₈alkylene which is interrupted by one or more O, S, NR₆, CO, SO and/or SO₂, C₃-C₃₀cycloalkylene which is interrupted by one or more O, S, NR₆, CO, SO and/or SO₂, C₄-C₃₀cycloalkenylene which is interrupted by one or more O, S, NR₆, CO, SO and/or SO₂, C₂-C₁₂alkenylene which is interrupted by one or more O, S, NR₆, CO, SO and/or SO₂;
wherein the groups C₁-C₁₈alkylene, C₃-C₃₀cycloalkylene, C₂-C₁₂alkenylene, C₄-C₃₀cycloalkenylene, interrupted C₂-C₁₈alkylene, interrupted C₃-C₃₀cycloalkylene interrupted C₄-C₃₀cycloalkenylene and interrupted C₂-C₁₂alkenylene optionally are substituted by one or more Ar₂, OH, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy, and/or by benzoyloxy;
wherein all radicals D₃ and O₄ optionally additionally are substituted by a group having a —O—C-bond or a —O—Si-bond which cleaves upon the action of an acid;
Ar′₂ is phenylene, biphenylene, naphthylene or heteroarylene;
wherein the groups phenylene, biphenylene, naphthylene and heteroarylene optionally are substituted by one or more OH, C₁-C₁₈alkyl, C₁-C₁₀haloalkyl, halogen, NO₂, CN, C₁-C₁₈alkoxy, phenoxy, NR₄R₅, C₁-C₁₂alkylthio, C₁-C₁₈alkylsulfonyloxy, phenylsulfonyloxy, (4-methylphenyl)sulfonyloxy, C₂-C₁₈alkanoyloxy and/or by benzoyloxy;
R₂ₐ has one of the meanings given for R₂;
provided that at least one of the radicals R₂, R₃, Ar₁ or Ar₁′ comprises a group of the formula IV.

2. A compound of the formula I, II or III according to claim 1, wherein
R₁ is C₁-C₁₈alkylsulfonyl, C₁-C₁₀haloalkylsulfonyl;
or R₁ is phenylsulfonyl which optionally are substituted by one or more C₁-C₁₈alkyl, C₁-C₁₀haloalkyl, halogen or NO₂;
R′₁ is phenylenedisulfonyl or C₁-C₁₀haloalkylenedisulfonyl;
R₂ is CN or C₁-C₁₀haloalkyl;
Ar₁ is phenyl, fluorenyl, naphthyl or heteroaryl,
all of which optionally are substituted by one or more OR₃, NR₄R₅, SR₇ or a group of formula IV 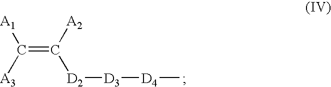
R₂ₐ has one of the meanings given for R₂;
Ar′₁ is phenylene or heteroarylene which optionally is substituted by a group of formula IV 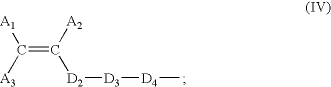
or Ar₁ is —Ar″₁—X₁—Y₁—X₁—Ar″₁—;
Ar″₁ is phenylene or naphthylene;
X₁ is O, NR₆ or S;
Y₁ is C₁-C₁₈alkylene which optionally is substituted by a group of formula IV 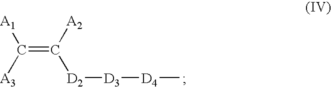
R₃ is C₁-C₁₈alkyl which optionally is substituted by a group of formula IV 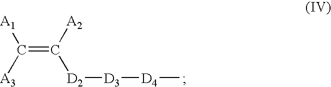
R₄ and R₅ independently of each other are hydrogen, C₁-C₁₈alkyl which optionally is substituted by a group of formula IV 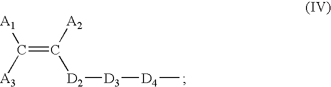
A₁, A₂ and A₃ independently of each other are hydrogen or C₁-C₁₈alkyl;
D₂ is (CO)O, Ar′₂, C₁-C₁₈alkylene;
or A₃ and D₂ together with the ethylenically unsaturated double bond to which they are attached form C₃-C₃₀cycloalkenyl which optionally is interrupted by one or more one or more N or CO;
or A₂ and D₂ together with the carbon of the ethylenically unsaturated double bond to which they are attached form C₃-C₃₀cycloalkyl which optionally is interrupted by one or more N or CO;
D3 and D₄ independently of each other are a direct bond, O, S, CO, O(CO), (CO)O, Ar′₂, 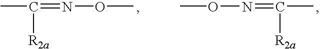
C₃-C₃₀cycloalkylene, C₁-C₁₈alkylene, C₂-C₁₈alkylene which is interrupted by one or more O, CO, NR₆ and/or SO₂;
R₆ is hydrogen; and
Ar′₂ is phenylene.

3. A compound of the formula I according to claim 1, wherein
R₁ is C₁-C₁₀haloalkylsulfonyl; or R₁ is phenylsulfonyl which optionally is substituted by C₁-C₁₀haloalkyl or NO₂;
R₂ is C₁-C₁₀haloalkyl;
Ar₁ is phenyl fluorenyl, naphthyl or heteroaryl, all of which are substituted by a group of formula IV 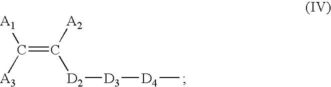
A₁, A₂ and A₃ independently of each other are hydrogen or C₁-C₄alkyl; and
D₂ is (CO)O;
D3 and D4 independently of each other are a direct bond, (CO)O, O, 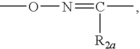
C₂-C₁₈alkylene or C₂-C₁₈alkylene which is interrupted by one or more CO or NR₆;
R₂ₐ is CN; and
R₆ is hydrogen.

4. A polymer comprising at least one repeating unit derived from the compound of the formula I, II and/or III according to claim 1.

5. A polymer according to claim 4 additionally to the at least one repeating unit derived from the compound of the formula I, II and/or III according to claim 1 comprising one or more identical or different repeating units derived from ethylenically unsaturated compounds selected from the group of formula V 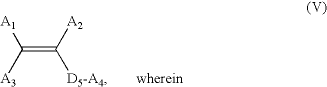
A₁, A₂ and A₃ independently of each other are hydrogen, halogen, CN, C₁-C₁₈alkyl, C₁-C₁₈alkyl which is substituted by OR₃; or A₁, A₂ and A₃ independently of each other are C₁-C₁₀haloalkyl, (CO)R₇, (CO)OR₃, or (CO)NR₄R₅;
A4 is C₁-C₁₈alkyl, C₂-C₁₈alkyl which is interrupted by one or more O, S, NR₆, CO, SO and/or SO₂,
C₃-C₃₀cycloalkyl, C₃-C₃₀cycloalkyl which is interrupted by one or more O, S, NR₆, CO, SO and/or SO₂,
C₂-C₁₂alkenyl, C₂-C₁₂alkenyl which is interrupted by one or more O, S, NR₆, CO, SO and/or SO₂,
C₄-C₃₀cycloalkenyl, C₄-C₃₀cycloalkenyl which is interrupted by one or more O, S, NR₆, CO, SO and/or SO₂,
wherein the groups C₁-C₁₈alkyl, interrupted C₂-C₁₈alkyl, C₃-C₃₀cycloalkyl, interrupted C₃-C₃₀cycloalkyl, C₂-C₁₂alkenyl, interrupted C₂-C₁₂alkenyl, C₄-C₃₀cycloalkenyl and interrupted C₄-C₃₀cycloalkenyl optionally are substituted by one or more Ar₂, OR₃, (CO)OR₃, O(CO)R₇, halogen, NO₂, CN, NR₄R₆ C₁-C₁₂alkylthio, C₁-C₁₈alkylsulfonyloxy, phenylsulfonyloxy, and/or (4-methylphenyl)sulfonyloxy;
or A₄ is hydrogen, halogen, NO₂, CN, Ar₂, (CO)R₇, (CO)OR₃, (CO)NR₄R₅, O(CO)R₇, O(CO)OR₃, O(CO)NR₄R₅, NR₆(CO)R₇, NR₆(CO)OR₃, OR₃, NR₄R₅, SR₆, SOR₂, SO₂R₇ and/or OSO₂R₇,
D₅ is a direct bond, O, CO, (CO)O, (CO)S, (CO)NR₆, SO₂, or OSO₂;
or D₅ is C₁-C₁₈alkylene;
or D₅ is a group Ar′₂;
optionally the radicals A₃ and D₅, together with the ethylenically unsaturated double bond to which they are attached form C₃-C₃₀cycloalkenyl which optionally is interrupted by one or more O, S, NR₆, CO, SO and/or SO₂;
or optionally the radicals A₂ and D₅ together with the carbon atom of the ethylenically unsaturated double bond to which they are attached form C₃-C₃₀cycloalkyl which optionally is interrupted by one or more O, S, N, NR₆, CO, SO and/or SO₂; and
R₃, R₄, R₅, R₆, R₇, Ar′₂ and Ar₂ are as defined in claim 1.

6. A polymer according to claim 5, wherein
A₁, A₂ and A₃ independently of each other are hydrogen or C₁-C₁₈alkyl;
A₄ is hydrogen, C₃-C₃₀cycloalkyl, C₃-C₃₀cycloalkyl which is interrupted by one or more O and/or CO, C₁-C₁₈alkyl, C₂-C₁₈alkyl which is interrupted by one or more O and/or CO, C₄-C₃₀cycloalkenyl which is interrupted by one or more O and/or CO;
wherein the groups C₃-C₃₀cycloalkyl, interrupted C₃-C₃₀cycloalkyl, C₁-C₁₈alkyl, interrupted C₂-C₁₈alkyl, and interrupted C₄-C₃₀cycloalkenyl optionally are substituted by one or more OR₃, (CO)OR₃ or O(CO)R₇;
D₅ is (CO)O;
R₃ is C₁-C₁₈alkyl, C₂-C₁₈alkyl which is interrupted by one or more O and/or CO, C₃-C₃₀cycloalkyl, C₃-C₃₀cycloalkyl which is interrupted by one or more O and/or CO, or is C₄-C₃₀cycloalkenyl which is interrupted by one or more O and/or CO;
or R₃ is hydrogen;
R₇ is C₃-C₃₀cycloalkyl, C₁-C₁₈alkyl, C₂-C₁₈alkyl which is interrupted by one or more O and/or CO, C₃-C₃₀cycloalkyl which is interrupted by one or more O and/or CO, C₄-C₃₀cycloalkenyl which is interrupted by one or more O and/or CO,
or R₇ is hydrogen.

7. A chemically amplified photoresist composition comprising
(a) a compound which cures upon the action of an acid; or a compound whose solubility is increased upon the action of an acid; and/or
(b) at least one compound of the formula I, II and/or III according to claim 1; and/or a polymer according to claim 4.

8. A chemically amplified photoresist composition according to claim 7, which is a positive resist.

9. A chemically amplified positive photoresist composition according to claim 8, comprising
(b) at least one polymer according to claim 4.

10. A chemically amplified positive photoresist composition according to claim 8, comprising
(b) at least one polymer comprising at least one repeating unit derived from a compound of the formula I, II and/or III according to claim 1; and
at least one repeating unit derived from ethylenically unsaturated compounds selected from the group of formula V as defined in claim 5; and
at least one repeating unit derived from ethylenically unsaturated compounds selected from the group of formula VI, 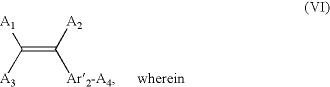
A₁, A₂, A₃, A₄ and Ar′₂ are as defined in claim 5.

11. A chemically amplified positive photoresist composition according to claim 8, comprising
(a1) at least one polymer having an acid-labile group which decomposes in the presence of an acid to increase the solubility in aqueous alkaline developer solution; and/or
(a2) at least one monomeric or oligomeric dissolution inhibitor having an acid-labile group which decomposes in the presence of an acid to increase the solubility in aqueous alkaline developer solution; and/or
(a3) at least one alkali-soluble monomeric, oligomeric or polymeric compound; and
(b) at least one compound of the formula I, II and/or III according to claim 1; and/or a polymer according to claim 4.

12. A chemically amplified positive photoresist composition according to claim 8, comprising
(a1) at least one polymer having an acid-labile group which decomposes in the presence of an acid to increase the solubility in aqueous alkaline developer solution; and/or
(a2) at least one monomeric or oligomeric dissolution inhibitor having an acid-labile group which decomposes in the presence of an acid to increase the solubility in aqueous alkaline developer solution; and/or
(a3) at least one alkali-soluble monomeric, oligomeric or polymeric compound; and
(b) at least one compound of the formula I, II and/or III according to claim 1; and/or a polymer comprising at least one repeating unit derived from the compound of the formula I, II and/or III according to claim 1 and at least one repeating unit derived from ethylenically unsaturated compounds selected from the group of formula VI as defined in claim 10, and optionally repeating units derived from ethylenically unsaturated compounds selected from the group of formula V as defined in claim 5.

13. A chemically amplified photoresist composition according to claim 7, which is a negative resist.

14. A chemically amplified negative photoresist composition according to claim 13, comprising
(a5) a component which, when catalysed by an acid undergoes a crosslinking reaction with itself and/or with the other components; and
(b) at least one compound of the formula I, II and/or III according to claim 1; and/or polymer comprising at least one repeating unit derived from the compound of the formula I, II and/or III according to claim 1 and optionally repeating units derived from ethylenically unsaturated compounds selected from the group of formula V as defined in claim 5.

15. A chemically amplified negative photoresist composition according to claim 13, comprising
(a4) an alkali-soluble resin as binder;
(a5) a component which, when catalysed by an acid undergoes a crosslinking reaction with itself and/or with the binder; and
(b) at least one compound of the formula I, II and/or III according to claim 1; and/or polymer comprising at least one repeating unit derived from the compound of the formula I, II and/or III according to claim 1 and optionally repeating units derived from ethylenically unsaturated compounds selected from the group of formula V as defined in claim 5.

16. A chemically amplified photoresist composition according to anyone of claims 7-15, in addition to components (b), or components (a1), (a2), (a3) and (b), or components (a5) and (b), or components (a4), (a5) and (b) comprising further additives (c), further photosensitive acid donor compounds (b1), other photoinitiators (d), and/or sensitizers (e).

17. A process for the preparation of a photoresist by
(1) applying to a substrate a composition according to claim 7;
(2) post apply baking the composition at temperatures between 60° C. and 160° C.;
(3) image-wise irradiating with light of wavelengths between 10 nm and 1500 nm;
(4) optionally post exposure baking the composition at temperatures between 60° C. and 160° C.; and
(5) developing with a solvent or with an aqueous alkaline developer.

18. A composition comprising
(b) at least one polymer comprising at least one repeating unit derived from the compound of the formula I, II and/or III according to claim 1 and repeating units derived from ethylenically unsaturated compounds selected from the group of formula V as defined in claim 5.

19. A composition comprising
(a) a compound which cures upon the action of an acid or a compound whose solubility is increased upon the action of an acid; and
(b) at least one compound of the formula I, II and/or III according to claim 1; and/or a polymer comprising at least one repeating unit derived from the compound of the formula I, II and/or III according to claim 1 and optionally repeating units derived from ethylenically unsaturated compounds selected from the group of formula V as defined in claim 5.

20. Use of compounds of the formula I, II or III according to claim 1; or polymers comprising at least one repeating unit derived from the compound of the formula I, II and/or III according to claim 1 and optionally repeating units derived from ethylenically unsaturated compounds selected from the group of formula V as defined in claim 5 as photosensitive acid donors in compositions that can be crosslinked under the action of an acid and/or as dissolution enhancers in compositions wherein the solubility is increased under the action of an acid.

21. Process for crosslinking compounds that can be crosslinked under the action of an acid or compounds whose solubility is increased upon the action of an acid, which method comprises adding a compound of the formula I, II and/or III according to claim 1; and/or polymer comprising at least one repeating unit derived from the compound of the formula I, II and/or III according to claim 1 and optionally repeating units derived from ethylenically unsaturated compounds selected from the group of formula V as defined in claim 5, to the above-mentioned compositions and irradiating imagewise or over the whole area with light having a wavelength of 10-1500 nm.

22. Use of compounds of the formula I, II or III according to claim 1; or polymers comprising at least one repeating unit derived from the compound of the formula I, II and/or III according to claim 1 and optionally repeating units derived from ethylenically unsaturated compounds selected from the group of formula V as defined in claim 5, as photosensitive acid donors in the preparation of surface coatings, printing inks, printing plates, dental compositions, colour filters, resists or image-recording materials, or image-recording materials for recording holographic images.

23. Process according to claim 21 for the preparation of surface coatings, printing inks, printing plates, dental compositions, colour filters, resists, or image-recording materials, or image-recording materials for recording holographic images.

24. Use of compounds of the formula I, II or III according to claim 1; or polymers comprising at least one repeating unit derived from the compound of the formula I, II and/or III according to claim 1 and optionally repeating units derived from ethylenically unsaturated compounds selected from the group of formula V as defined in claim 5, as photosensitive acid donors in the preparation of colour filters or chemically amplified resists.

25. Process according to claim 21 for the preparation of colour filters or chemically amplified resists.

26. A color filter prepared by providing red, green and blue picture elements and a black matrix, all comprising a photosensitive resin and a pigment and/or dye on a transparent substrate and providing a transparent electrode either on the surface of the substrate or on the surface of the color filter layer, wherein said photosensitive resin comprises polymers comprising at least one repeating unit derived from the compound of the formula I, II and/or III according to claim 1 and optionally repeating units derived from ethylenically unsaturated compounds selected from the group of formula V as defined in claim 5, as photosensitive acid donors.
